(19) United States
(12) Patent Application Publication
(10) Pub. No.: US 2014/0322771 A1
(43) Pub. Date: Oct. 30, 2014

(54) Lipid Production

(71) Applicants: (Robert) Hongyuan Yang, Matraville (AU); Junmin Pan, Beijing (CN); Jacqueline Batley, St. Lucia (AU)

(72) Inventors: (Robert) Hongyuan Yang, Matraville (AU); Junmin Pan, Beijing (CN); Jacqueline Batley, St. Lucia (AU)

(73) Assignee: THE UNIVERSITY OF QUEENSLAND, Queensland (AU)

(21) Appl. No.: 13/992,444

(22) PCT Filed: Dec. 9, 2011
(86) PCT No.: PCT/AU11/01600

§ 371 Date: Jul. 17, 2014

(30) Foreign Application Priority Data
Dec. 9, 2010 (AU) 2010905431
Jul. 22, 2011 (AU) 2011902937

(51) Int. Cl. C12P 7/64; C12N 15/63

(52) U.S. Cl. CPC C12P 7/649; C12P 7/6445; C12N 15/63
USPC 435/134; 435/471; 435/468

(57) ABSTRACT

The invention relates to methods for producing lipids and in particular methods for producing glycerolipids suitable for use in generating biofuels. The glycerolipids are produced through the modification of a cell so as to increase phosphatidic acid via the inhibition of the phosphatidylethanolamine N-methyltransferase (PEMT) pathway. The invention further relates to genetically modified plants and microorganisms in which the production of glycerolipids/oil is increased.

### INCORPORATION BY CROSS-REFERENCE

This application claims priority from Australian provisional patent application number 2010905431 (AU 2010905431) filed on 9 Dec. 2010 and Australian provisional patent application number 2011902937 (AU 2011902937) filed on 22 Jul. 2011. The entire contents of AU 2010905431 and AU 2011902937 are incorporated herein by cross-reference.

### TECHNICAL FIELD

The invention relates to methods for producing lipids and in particular methods for producing glycerolipids suitable for use in generating biofuels. The invention further relates to genetically modified plants and microorganisms in which the production of glycerolipids/oil is increased.

### BACKGROUND

Biofuels (e.g. biodiesel) may be derived from lipid-containing vegetable oils such as soybean oil and palm oil. However, given the current food crisis alternative sources of vegetable oils for biofuel production need to be identified to reduce the reliance on edible plants. Photosynthetic microorganisms (e.g. microalgae) are a promising alternative for the production of the vegetable oils as they possess high lipid content and do not require arable land. The direct use of vegetable oils derived from plants and microorganisms such as algae in currently available diesel engines is impractical predominantly due to issues concerning viscosity, volatility and acid contamination. Therefore, lipids in vegetable oils derived from these sources must be processed to acquire properties similar to that of petrodiesel. A commonly used technique of producing biodiesel is the transesterification of triacylglycerols (TAGs), the primary lipid component of vegetable oils. Both the yield and quality of vegetable oil TAGs from the source organism utilised are critical factors in biodiesel production. For example, it is preferable to produce TAGs with shorter and saturated acyl chains because the resulting fatty acids possess more favourable properties as transportation fuels.

There is a need for improved methods of generating lipids suitable for biofuel production. A need also exists for plants and microorganisms with characteristics that improve the yield and/or quality of lipids suitable for biofuel production.

### SUMMARY OF THE INVENTION

In a first aspect, the invention provides a method for increasing triacylglycerol production in a cell, the method comprising modifying the cell to increase phosphatidic acid production.

In a second aspect, the invention provides a method for increasing the energry-storage potential of a cell, the method comprising modifying the cell to increase phosphatidic acid production.

In one embodiment of the second aspect, the method comprises fusing multiple lipid droplets within the cell.

In one embodiment of the first or second aspect, the modifying comprises inhibiting the phosphatidylethanolamine N-methyltransferase (PEMT) pathway in said cell.

In another embodiment of the first or second aspect, the inhibiting comprises reducing the quantity or activity of an enzyme, enzyme cofactor, precursor compound, or intermediate compound of said PEMT pathway.

In a further embodiment of the first or second aspect, the PEMT pathway enzyme is phosphatidylethanolamine methyltransferase.

In an additional embodiment of the first or second aspect, the PEMT pathway enzyme is phospholipid methyltransferase.

In one embodiment of the first or second aspect, the inhibiting comprises reducing the quantity or activity of transcription factor that regulates a PEMT pathway protein.

In a further embodiment of the first aspect, the transcription factor regulates inositol-1-phosphate synthase expression.

In one embodiment of the first or second aspect, the inhibiting comprises reducing the quantity or activity of a protein in the cell, wherein said protein is selected from the group consisting of: inositol-1-phosphate synthase, CDP-diacylglycerol synthase (phosphatidate cytidylyltransferase), (e.g. CDP-diacylglycerol synthase 1, CDP-diacylglycerol synthase 2), casein kinase II, a beta regulatory subunit of casein kinase II, a protein homologous to Saccharomyces cerevisiae INO2/YDR123C, a protein homologous to Saccharomyces cerevisiae INO4/YOL108C, a protein homologous to Saccharomyces cerevisiae RTC2/YBR147W, a protein homologous to Saccharomyces cerevisiae MRPS35/YGR165W, a protein homologous to Saccharomyces cerevisiae FLD1/YLR404W, and a protein homologous to Saccharomyces cerevisiae FLD1/YLR404W.

In another embodiment of the first or second aspect, the inhibiting comprises inhibiting the expression of a gene encoding a protein selected from the group consisting of: inositol-1-phosphate synthase, CDP-diacylglycerol synthase (phosphatidate cytidylyltransferase) (e.g. CDP-diacylglycerol synthase 1, CDP-diacylglycerol synthase 2), casein kinase II, a beta regulatory subunit of casein kinase II, a protein homologous to Saccharomyces cerevisiae INO2/YDR123C, a protein homologous to Saccharomyces cerevisiae INO4/YOL108C, a protein homologous to Saccharomyces cerevisiae RTC2/YBR147W, a protein homologous to Saccharomyces cerevisiae MRPS35/YGR165W, a protein homologous to Saccharomyces cerevisiae FLD1/YLR404W, and a protein homologous to Saccharomyces cerevisiae FLD1/YLR404W.

In one embodiment of the first or second aspect, the inhibiting comprises reducing the quantity or activity of a CDP-diacylglycerol synthase protein encoded by a gene comprising a sequence as defined in SEQ ID NO: 41.

In one embodiment of the first or second aspect, the inhibiting comprises reducing the quantity or activity of phosphatidylethanolamine methyltransferase and phospholipid methyltransferase.

In one embodiment of the first or second aspect, the inhibiting comprises reducing the quantity or activity of phosphatidylethanolamine methyltransferase, phospholipid methyltransferase, and a protein homologous to Saccharomyces cerevisiae FLD1/YLR404W.

In another embodiment of the first or second aspect, the inhibiting comprises reducing the quantity or activity of phosphatidylethanolamine methyltransferase, phospholipid methyltransferase and CDP-DAG synthase.

In one embodiment of the first or second aspect, the inhibiting comprises reducing the quantity or activity of phosphatidylethanolamine methyltransferase, phospholipid methyltransferase, and casein kinase 2.

In another embodiment of the first or second aspect, the inhibiting comprises reducing the quantity or activity of phosphatidylethanolamine methyltransferase, phospholipid methyltransferase, CDP-DAG synthase and casein kinase 2.

In a further embodiment of the first or second aspect, the cell is a bacterial cell, a yeast, an algal cell, or a plant cell.

In a further embodiment of the first or second aspect, the cell is a Chlamydomonas sp. microorganism.

In another embodiment of the first or second aspect, the method comprises inhibiting the expression of a gene in said cell.

In one embodiment of the first or second aspect, the organism is a non-human organism.

In a third aspect, the invention provides a method for producing a genetically modified organism, the method comprising making at least one genetic modification to a cell of the organism that increases phosphatidic acid production in the cell, wherein triacylglycerol production in the cell is elevated compared to a corresponding wild-type cell, and said organism is a microorganism or plant.

In one embodiment of the third aspect, the genetic modification inhibits the phosphatidylethanolamine N-methyltransferase (PEMT) pathway in said cell.

In another embodiment of the third aspect, the genetic modification reduces the quantity or activity of an enzyme, enzyme cofactor, precursor compound, or intermediate compound of said PEMT pathway.

In a further embodiment of the third aspect, the PEMT pathway enzyme is phosphatidylethanolamine methyltransferase.

In an additional embodiment of the third aspect, the PEMT pathway enzyme is phospholipid methyltransferase

In one embodiment of the third aspect, the genetic modification reduces the quantity or activity of a transcription factor that regulates a PEMT pathway protein.

In a further embodiment of the third aspect, the transcription factor regulates inositol-1-phosphate synthase expression.

In another embodiment of the third aspect, the genetic modification reduces the quantity or activity of a protein selected from the group consisting of: inositol-1-phosphate synthase, CDP-diacylglycerol synthase (phosphatidate cytidylyltransferase), (e.g. CDP-diacylglycerol synthase 1, CDP-diacylglycerol synthase 2), casein kinase II, a beta regulatory subunit of casein kinase II, a protein homologous to Saccharomyces cerevisiae INO2/YDR123C, a protein homologous to Saccharomyces cerevisiae INO4/YOL108C, a protein homologous to Saccharomyces cerevisiae RTC2/YBR147W, a protein homologous to Saccharomyces cerevisiae MRPS35/YGR165W, a protein homologous to Saccharomyces cerevisiae FLD1/YLR404W, and a protein homologous to Saccharomyces cerevisiae FLD1/YLR404W.

In another embodiment of the third aspect, the genetic modification inhibits the expression of a gene in the organism encoding a protein selected from the group consisting of: inositol-1-phosphate synthase, CDP-diacylglycerol synthase (phosphatidate cytidylyltransferase) (e.g. CDP-diacylglycerol synthase 1, CDP-diacylglycerol synthase 2), casein kinase II, a beta regulatory subunit of casein kinase II, a protein homologous to Saccharomyces cerevisiae INO2/YDR123C, a protein homologous to Saccharomyces cerevisiae INO4/YOL108C, a protein homologous to Saccharomyces cerevisiae RTC2/YBR147W, a protein homologous to Saccharomyces cerevisiae MRPS35/YGR165W, a protein homologous to Saccharomyces cerevisiae FLD1/YLR404W, and a protein homologous to Saccharomyces cerevisiae FLD1/YLR404W.

In one embodiment of the third aspect, the genetic modification reduces the quantity or activity of a CDP-diacylglycerol synthase protein encoded by a gene comprising a sequence as defined in SEQ ID NO: 41.

In one embodiment of the third aspect, the genetic modification reduces the quantity or activity of phosphatidylethanolamine methyltransferase and phospholipid methyltransferase.

In another embodiment of the third aspect, the genetic modification reduces the quantity or activity of phosphatidylethanolamine methyltransferase, phospholipid methyltransferase and CDP-DAG synthase.

In one embodiment of the third aspect, the genetic modification reduces the quantity or activity of phosphatidylethanolamine methyltransferase, phospholipid methyltransferase, and casein kinase 2.

In another embodiment of the third aspect, the genetic modification reduces the quantity or activity of phosphatidylethanolamine methyltransferase, phospholipid methyltransferase, CDP-DAG synthase and casein kinase 2.

In a further embodiment of the third aspect, the genetically modified organism is a bacterium, plant or alga.

In a further embodiment of the third aspect, the genetically modified organism is a Chiamydomonas sp. organism. In a fourth aspect, the invention provides a method for producing a biofuel, the method comprising:
cultivating a genetically modified organism according to the second aspect, isolating triacylglycerols produced by the organism; and
transesterifying the triacylglycerols to produce the biofuel.

In one embodiment of the fourth aspect, the transesterifying is performed using a monohydric alcohol and a base catalyst.

In one embodiment of the fourth aspect, the biofuel is biodiesel.

In a fifth aspect, the invention provides use of a genetically modified organism produced by the method of the third aspect in a process for producing a biofuel.

In a sixth aspect, the invention provides a method for increasing triacylglycerol production in a cell, the method comprising reducing the quantity or activity of a synaptobrevin/vesicle-associated membrane protein (VAMP)-associated protein in the cell.

In one embodiment of the sixth aspect, the VAMP-associated protein is a protein homologous to Saccharomyces cerevisiae SCS2/YER120W.

In another embodiment of the sixth aspect, the VAMP-associated protein comprises a sequence defined in SEQ ID NO: 5, SEQ ID NO: 17, SEQ ID NO: 37, SEQ ID NO: 38, SEQ ID NO: 39, or SEQ ID NO: 40.

In another embodiment of the sixth aspect, the quantity or activity of the VAMP-associated protein is reduced by inhibiting the expression of a gene encoding the VAMP in the cell.

In a further embodiment of the sixth aspect, the gene is a homologue of S. cerevisiae scs2.

In a further embodiment of the sixth aspect, the cell is a bacterium, yeast, plant or algal cell.

In a seventh aspect, the invention provides a method for producing a genetically modified organism, the method comprising making at least one genetic modification to a cell of the organism that reduces the quantity or activity of a synaptobrevin/vesicle-associated membrane protein (VAMP)-associated protein in the cell, wherein said organism is a microorganism or plant.

In one embodiment of the seventh aspect, the VAMP-associated protein is a protein homologous to Saccharomyces cerevisiae SCS2/YER120W.

In another embodiment of the seventh aspect, the VAMP-associated protein comprises a sequence defined in SEQ ID NO: 5, SEQ ID NO: 17, SEQ ID NO: 37, SEQ ID NO: 38, SEQ ID NO: 39, or SEQ ID NO: 40.

In another embodiment of the seventh aspect, the genetic modification inhibits the expression of a gene encoding the VAMP-associated protein in the organism.

In a further embodiment of the seventh aspect, the genetically modified organism is a bacterium, yeast, plant or alga. In an eighth aspect, the invention provides a genetically modified organism produced in accordance with the method of the third aspect.

In a ninth aspect, the invention provides a genetically modified organism produced in accordance with the method of the seventh aspect.

In a tenth aspect, the invention provides use of a genetically modified organism produced by the method of the seventh aspect in a process for producing a biofuel.

In an eleventh aspect, the invention provides a biofuel produced in accordance with the method of the fourth aspect, the use of the fifth aspect, or the use of the tenth aspect.

In one embodiment of the eleventh aspect, the biofuel is biodiesel.

In a further embodiment of the first, second, or sixth aspect, the cell is Chlamydomonas reinhardtii.

In a further embodiment of the first, second, or sixth aspect, the cell is a Saccharomyces sp. microorganism.

In a further embodiment of the first, second, or sixth aspect, the cell is Saccharomyces cerevisiae.

In a further embodiment of the first, second, or sixth aspect, the cell is a plant cell.

In a further embodiment of the first, second, or sixth aspect, the cell is a plant cell selected from an Arabidposis sp. plant cell, a Glycine sp. plant cell, a Brassica sp. plant cell, and a Ricinus sp. plant cell.

In a further embodiment of the first, second, or sixth aspect, the cell is a plant cell selected from a Brassica rapa plant cell and an Arabidposis thaliana plant cell.

In a further embodiment of the third, seventh or eighth aspect, the genetically modified organism is Chlamydomonas reinhardtii.

In a further embodiment of the third, seventh or eighth aspect, the genetically modified organism is a Saccharomyces sp. microorganism.

In a further embodiment of the third, seventh or eighth aspect, the genetically modified organism is Saccharomyces cerevisiae.

In a further embodiment of the third, seventh or eighth aspect, the genetically modified organism is a plant.

In a further embodiment of the third, seventh or eighth aspect, the genetically modified organism is a plant selected from an Arabidposis sp. plant, a Glycine sp. plant, a Brassica sp. plant, and a Ricinus sp. plant.

In a further embodiment of the third, seventh or eighth aspect, the genetically modified organism is a plant selected from a Brassica rapa plant and an Arabidposis thaliana plant.

In another embodiment of the first, second, third, fourth, fifth or eighth aspect, the CDP-diacylglycerol synthase (phosphatidate cytidylyltransferase) protein comprises a sequence as set forth in any one of SEQ ID NO: 1, SEQ ID NO: 6, SEQ ID NOs 18-21, a variant of any one of said sequences, or a fragment of any one of said sequences.

In another embodiment of the first, second, third, fourth, fifth or eighth aspect, the casein kinase II protein comprises a sequence as set forth in any one of SEQ ID Nos 2-3, SEQ ID NOs 8-9, SEQ ID Nos 22-27, a variant of any one of said sequences, or a fragment of any one of said sequences.

In another embodiment of the first, second, third, fourth, fifth or eighth aspect, the protein homologous to Saccharomyces cerevisiae RTC2/YBR147W is a transmembrane family protein. The transmembrane family protein may be a PQ-loop repeat family protein.

In another embodiment of the first, second, third, fourth, fifth or eighth aspect, the protein homologous to Saccharomyces cerevisiae RTC2/YBR147W comprises a sequence as set forth in any one of SEQ ID NO: 4, SEQ ID NOs 13-16, SEQ ID NOs 29-36, a variant of any one of said sequences, or a fragment of any one of said sequences.

In another embodiment of the first, second, third, fourth, fifth or eighth aspect, the protein homologous to Saccharomyces cerevisiae INO2/YDR123C comprises a sequence as set forth in any one of SEQ ID NO: 10, a variant of SEQ ID NO: 10, or a fragment of SEQ ID NO: 10.

In another embodiment of the first, second, third, fourth, fifth or eighth aspect, the protein homologous to Saccharomyces cerevisiae MRPS35/YGR165W comprises a sequence as set forth in any one of SEQ ID NO: 11, a variant of SEQ ID NO: 11, or a fragment of SEQ ID NO: 11.

In another embodiment of the first, second, third, fourth, fifth or eighth aspect, the inhibiting comprises reducing the quantity or activity of a phospholipid N-methyltransferase protein in the cell.

In another embodiment of the first, second, third, fourth, fifth or eighth aspect, the phospholipid N-methyltransferase protein comprises a sequence as set forth in any one of SEQ ID NO: 12, SEQ ID NO: 28, a variant of any one of said sequences, or a fragment of any one of said sequences.

In another embodiment of the first, second, third, fourth, fifth or eighth aspect, the gene encoding a CDP-diacylglycerol synthase (phosphatidate cytidylyltransferase) protein comprises a sequence as set forth in any one of SEQ ID NO: 44, SEQ ID NO: 45, SEQ ID NO: 49, SEQ ID NO: 50, SEQ ID NO: 54, a variant of any one of said sequences, or a fragment of any one of said sequences,

In another embodiment of the first, second, third, fourth, fifth or eighth aspect, the gene encoding a casein kinase II protein comprises a sequence as set forth in any one of SEQ ID NO: 47, SEQ ID NO: 51, a variant of any one of said sequences, or a fragment of any one of said sequences,

In another embodiment of the first, second, third, fourth, fifth or eighth aspect, the gene encoding a protein homologous to Saccharomyces cerevisiae RTC2/YBR147W is a transmembrane family protein. The transmembrane family protein may be a PQ-loop repeat family protein.

In another embodiment of the first, second, third, fourth, fifth or eighth aspect, the gene encoding a protein homologous to Saccharomyces cerevisiae RTC2/YBR147W comprises a sequence as set forth in any one of SEQ ID NO: 46, SEQ ID NO: 52, a variant of any one of said sequences, or a fragment of any one of said sequences,

In another embodiment of the first, second, third, fourth, fifth or eighth aspect, the inhibiting comprises inhibiting the expression of a gene encoding a phospholipid N-methyltransferase protein in the cell.

In another embodiment of the sixth, seventh, ninth, or tenth aspect, the VAMP-associated protein or protein homologous to Saccharomyces cerevisiae SCS2/YER120W comprises a sequence as set forth in any one of SEQ ID NO: 5, SEQ ID NO: 17, SEQ ID NOs 37-40, a variant of any one of said sequences, or a fragment of any one of said sequences.

In another embodiment of the sixth, seventh, ninth, or tenth aspect, the gene encoding the VAMP-associated protein or protein homologous to Saccharomyces cerevisiae SCS2/YER120W comprises a sequence as set forth in any one of SEQ ID NO: 48, SEQ ID NO: 53, a variant of any one of said sequences, or a fragment of any one of said sequences.

In another embodiment of the first, second, fourth, fifth, sixth, eighth, or eleventh aspect, the triacylglycerol comprises at least one saturated fatty acid chain.

In an additional embodiment of the first, second, fourth, fifth, sixth, eighth, or eleventh aspect, the triacylglycerol comprises at least one short chain fatty acid comprising less than 14 carbon atoms.

In one embodiment of any one of the first to tenth aspects, the variant has a level of sequence identity with said sequence selected from the group consisting of at least 98% sequence identity, at least 95% sequence identity, at least 90% sequence identity, at least 85% sequence identity, at least 80% sequence identity, and at least 75% sequence identity,

In one embodiment of any one of the first to tenth aspects, the variant is a homologous gene aor a homologous protein in an organism of a different species, genus, family or class.

In one embodiment of any one of the first to tenth aspects, the fragment is less than 1000 base pairs in length, less than 900 base pairs in length, less than 800 base pairs in length, less than 700 base pairs in length, less than 600 base pairs in length, less than 500 base pairs in length, less than 400 base pairs in length, less than 300 base pairs in length, less than 200 base pairs in length, less than 100 base pairs in length, less than 75 base pairs in length, less than 50 base pairs in length, less than 40 base pairs in length, less than 30 base pairs in length, less than 20 base pairs in length, or less than 10 base pairs in length,

In one embodiment of any one of the first to tenth aspects, the fragment is less than 1000 amino acid residues in length, less than 900 amino acid residues in length, less than 800 amino acid residues in length, less than 700 amino acid residues in length, less than 600 amino acid residues in length, less than 500 amino acid residues in length, less than 400 amino acid residues in length, less than 300 amino acid residues in length, less than 200 amino acid residues in length, less than 100 amino acid residues in length, less than 75 amino acid residues in length, less than 50 amino acid residues in length, less than 40 amino acid residues in length, less than 30 amino acid residues in length, less than 20 amino acid residues in length, or less than 10 amino acid residues in length.

### BRIEF DESCRIPTION OF THE DRAWINGS

Preferred embodiments of the invention will now be described, by way of example only, with reference to the accompanying figures wherein:

FIG. 1 provides microscopy images and a graph showing yeast fld1Δ and nine additional mutant strains produce “supersized” LDs (SLDs). Cells were grown in YPD or SC medium until early stationary phase, stained with Nile red, and examined by fluorescence microscopy. Bar, 5 μm. (A) LDs of WT and fld1Δ. (B) LDs of mutant strains defective in either CDP-choline pathway (cki1Δ, pct1Δ, and cpt1Δ) or PEMT pathway (cho2Δ, opi3Δ, ino2Δ, and ino4Δ) of PC synthesis. (C) Supersized LDs observed in rtc2Δ, mrps35Δ, ckb1Δ and ckb2Δ. (D) LDs of a yeast strain with the CDS1 gene under the control of a tetracycline-regulated promoter grown in the presence or absence of doxycycline. (E) Transmission electron microscopic examination of LDs in WT, fld1Δ, cho2Δ, opi3Δ, ino2Δ, ino4Δ, and cds1 strains cultured in SC medium. Bar, 1 μm. (F) Relative cellular amounts of TAG and SE. #, p<0.05; *, p<0.01, compared to WT.

FIG. 2 provides microscopy images and a graph showing treatment of different phospholipid precursors exerts distinct effects on the formation of SLDs. WT and mutants were cultured in SC media supplemented without (con) or with 1 mM choline (C), 1 mM ethanolamine (E), or 75 μM inositol (I) to stationary phase and observed under a fluorescence microscope. (A) Microscopic images. Bar, 5 μm. (B) Percentage of cells containing SLDs. *, p<0.01.

FIG. 3 provides graphs illustrative of a link between the formation of SLDs and an elevated level of cellular PA. (A) and (B) Quantitation of cellular PA in WT and mutants. Cells were grown in SC medium to early stationary phase, harvested, and lyophilized. Lipids were extracted and PA levels were determined by LC-MS. *, p<0.05, compared to WT. (C) Overexpression of PAH1 and DPP1 significantly reduces the formation of SLDs in fld1Δ, ino2Δ, and ino4Δ strains. Cells transformed with BG1805-PAH1, BG1805-DPP1 (both from Open Biosystems) or empty vector were cultured in synthetic galactose medium (2% galactose, 0.67% yeast nitrogen base, and amino acids) to stationary phase, stained with Nile red, and examined for the presence of SLDs.

FIG. 4 shows graphs illustrating Fld1p and cellular PA expression. (A) The gene expression fold changes (fld1Δ/WT) for selective genes involved in phospholipid metabolism as determined by microarray analysis. Cells were cultured in YPD medium until log phase (OD₆₀₀˜0.8). The expression levels of INO1 and OPI3 were significantly upregulated in fld1Δ cells. *, p<0.01 (ANOVA, FDR <0.05). (B) Relative mRNA levels of INO1 and OPI3 as determined by qPCR in WT and fld1Δ strains and normalized to ACT1. *, p<0.01. (C) WT and fld1Δ cells without or with (+Ino) inositol treatment were grown to late log phase. Microsomes were isolated as described in methods. Lipids were extracted and the amounts of PA were determined by mass-spectrometry. #, p<0.05, compared to WT.

FIG. 5 provides a microscopy image and graphs illustrating SLD formation in yeast cells deficient in PA phosphatase activity. (A) and (B) Formation of SLDs in pah1Δ, but not in dga1Δ or dga1Δ lro1Δ strains. Bar, 5 μm. (C) Relative cellular TAG amounts quantified by thin layer chromatography and densitometry. (D) Addition of 1 mM membrane-permeable DAG analog (1,2-dioctanoyl-sn-glycerol) and 1 mM oleate, but not oleate alone, significantly elevated the percentage of pah1Δ cells accumulating. supersized LDs. Cells with supersized LDs were counted and the percentages were presented as mean±SD.

FIG. 6 provides graphs showing (A) PE/PL ratio of LDs isolated from WT and mutants grown in SC medium, and of LDs isolated from ino4Δ, fld1Δ, and cds1 cells cultured in SC medium without (con) or with the addition of choline (C), ethanolamine (E), or inositol (I); (B) PL/TAG ratio of LDs isolated from WT and mutants grown in SC medium or YPD medium, and of LDs isolated from ino4Δ, fld1Δ, and cds1 cells cultured in SC medium without (con) or with the addition of choline (C), ethanolamine (E), or inositol (I). *, p<0.01; **, p<0.05, compared to WT.

FIG. 7 provides graphs showing rtc2Δ and mrps35Δ strains exhibit a higher PE/PC ratio. WT and mutants were cultured in SC medium to early stationary phase. Cellular amounts of major phospholipid species were analyzed by LC-MS. #, p<0.05; *, p<0.01, compared to WT.

FIG. 8 provides microscopy images showing that mutant strains that produce SLDs display enhanced LD fusion activity in vivo. Cells were grown in SC media until mid-log phase (OD₆₀₀˜1.5), stained with Nile red and examined for LD fusion activities under fluorescence microscope. Cells in which two or several LDs lay close together were targeted. Images were taken at a 0.5 s interval. Bar, 5 μm.

FIG. 9 provides a graph indicative of the stability and size of TAG containing artificial droplets consisting of the indicated molar ratios of the phospholipids were determined by light scattering. The artificial droplets were generated by sonication, and liposomes formed at the same time were removed by density gradient centrifugation. The stability/number of LDs was measured by light scattering. Values are mean±SD of three experiments.

FIG. 10 is a diagram showing biosynthetic pathways of major phospholipids and TAG in S. cerevisiae. PA, phosphatidic acid; CDP-DAG, CDP-diacylglycerol; PI, phosphatidylinositol; PS, phosphatidylserine; PE, phosphatidylethanolamine; PMME, phosphatidylmonomethylethanolamine; PDME, phosphatidyldimethylethanolamine; PC, phosphatidylcholine; DAG, diacylglycerol; TAG, triacylglycerol. Grey-shaded enzymes are under the control of the Ino2p-Ino4p complex

FIG. 11 provides microscopy images and a timecourse graph showing in vivo TAG mobilization of WT, fld1Δ, ino4Δ, and cds1 in the presence of 10 μg/ml cerulenin. Cells were grown in SC medium for 24 hr, and refreshed in YPD medium supplemented with 10 μg/ml cerulenin to OD600˜1.5. Cells were collected at indicated time points, followed by fluorescence microscopy and lipid analysis.

FIG. 12 provides graphs indicating the effect of DPP1 and PAH1 expression on cellular lipids. (A) Total cellular PA as measured by mass-spectrometry. *, p<0.01, compared to vector control. (B) Major phospholipids on lipid droplets measured by mass-spectrometry.

FIG. 13 shows relative cellular levels of PC, PE and PI in WT and mutants as determined by thin layer chromatography (TLC). Densitometric analysis was performed using the Image Gauge 4.0 software (Fujifilm Science Lab).

FIG. 14 provides fluorescent microscopy images showing lipid droplet accumulation in the algae Chlamydomonas. (A) wild type (B) transformed with RNAi construct.

### DEFINITIONS

As used in this application, the singular form “a”, “an” and “the” include plural references unless the context clearly dictates otherwise. For example, the term “a plant cell” also includes a plurality of plant cells.

As used herein, the term “comprising” means “including.” Variations of the word “comprising”, such as “comprise” and “comprises,” have correspondingly varied meanings. Thus, for example, a polynucleotide “comprising” a sequence of nucleotides may consist exclusively of that sequence, or, may include one or more additional sequences of nucleotides.

As used herein, the term “biofuel” includes any energy-containing fuel product derived from the processing of a lipid. Non-limiting examples of biofuels include oil products (i.e. bio-oils), biodiesel, and alcohols (e.g. ethanol and butanol).

Any description of prior art documents herein, or statements herein derived from or based on those documents, is not an admission that the documents or derived statements are part of the common general knowledge of the relevant art.

For the purposes of description all documents referred to herein are incorporated by reference in their entirety unless otherwise stated.

### DETAILED DESCRIPTION

A number of current methods for biofuel production utilise lipids generated by plants and/or microorganisms (e.g. algae and bacteria). The economic feasibility of current methods depends largely on the yield and/or characteristics of lipids harvested from the plants and/or microorganisms utilised.

The present inventors have identified that the quantity and/or quality of lipids in plants and microorganisms can be improved by raising intracellular levels of phosphatidic acid. In particular, increasing phosphatidic acid has been identified to cause increased production of glycerolipids (e.g. triacylglycerols and sterol esters) including those comprising short and/or saturated acyl chains. The present inventors have also identified that increased cellular phosphatidic acid is strongly associated with the formation of large/supersized lipid droplets within a cell. Without limitation to a particular mode of action, it is postulated that increasing cellular levels of phosphatidic acid serves to enhance the fusion properties of intracellular lipid droplets resulting in the formation of large/supersized lipid droplets within cells. These large/supersized lipid droplets contain large numbers of certain lipids (e.g. glycerolipids including triacylglycerols) with characteristics advantageous for biofuel production (e.g. short and/or saturated acyl chains). Further, large/supersized lipid droplets provide a much more efficient way for the cell to store energy in comparison to having a large number of small lipid droplets.

Accordingly, the invention provides methods for increasing the production of lipids in a plant or microorganism, the methods comprising increasing cellular levels of phosphatidic acid. Methods for increasing the energy-storage potential of a cell in a plant or microorganism are also provided, the methods comprising increasing cellular levels of phosphatidic acid.

The generation of plants and microorganisms with improved lipid-producing qualities is desirable to increase the efficiency of biofuel production and enhance its utility as a replacement for fossil fuels. To this end, the invention provides genetically modified plants and microorganisms in which the quantity and/or quality of lipids (e.g. triacylglycerols and sterol esters) is increased compared to corresponding wild-type microorganisms or plants. Genetically modified plants and microorganisms of the invention generally comprise at least one genetic alteration that increases cellular levels of phosphatidic acid.

Additional aspects of the invention relate to methods for generating biofuels (e.g. biodiesel) utilising lipids produced by genetically modified plants and/or microorganisms described herein.

### Microorganisms and Plants

The invention provides methods for increasing the production of lipids in a cell. In some embodiments, the lipids are glycerolipids (e.g. triacylglycerols and sterol esters). In some embodiments, the glycerolipids comprise short and/or saturated acyl chains.

The cell may be any cell capable of lipid production. For example, the cell may be a lipid-producing unicellular organism (e.g. a bacterium, microalga or fungus). Alternatively, the cell may be derived from or exist as a constituent of a lipid-producing multicellular organism (e.g. a plant).

Accordingly, the methods of the invention may be used to increase lipid production in a plant cell. Non-limiting examples of plant cells in which lipid production may be increased include Azadirachta indica (neem), Brassica sp. (mustard), Carapa sp. (andiroba), Orbignia sp (babassu), Hordeum vulgare (barley), Ricinus communis (castor), Camelina saliva (camelina), Brassica campestris (canola/rapeseed), Cocos nucifera (coconut), Zea mays (corn), Dipteryx odorata (cumaru), Cynara cardunculus (artichoke thistle), Arachis hypogaea (groundnut), Pongamia glabra (karanja), Jatropha curcas (jatropha), Lauyrus sp. (bay laurel), Lesquerella fendleri (lesquerella), Caryocar sp. (piqui), Elaeis sp. (palm), Sesamum indicum (sesame), Sorghum sp. (grasses) and Glycine max (soybean) species.

The methods may be used to increase lipid production in a bacterial cell. Non-limiting examples of bacteria in which lipid production may be increased include Rhodococcus sp. (e.g. R. opacus, R. fascians, R. erythroplois, R. glutinis, Nocardia sp.), (e.g. N. corallina, N. asteroids, N. globerula, N. restricta), Gordonia sp. (e.g. Gordonia amarae), Pseudomonas sp. (e.g. P. aeruginosa), Acinetobacter sp. (e.g. A. lwoffii) and cyanobacteria.

The methods may be used to increase lipid production in a fungal cell. Non-limiting examples of fungi in which lipid production may be increased include Candida sp. (e.g. C. valida, C. utilus), Yarrowia sp. (e.g. Y. lypolytica), Rhodotorula (e.g. R. gracilis) and Mortiorella sp. (e.g. M. peryronel).

The methods may be used to increase lipid production in an algal cell. Preferably, the algal cell is a microalgal cell. Non-limiting examples of algae in which lipid production may be increased include Chlorophytes (e.g. Ankistrodesmus sp., Botryococcus sp., Chlamydomonas sp. (including C. reinhardtii), Chlorella sp., Chlorococcum sp., Dunaliella sp., Monoraphidium sp., Oocystis sp., Scenedesmus sp., and Tetraselmis sp.), cyanophytes (e.g. Oscillatoria sp. and Synechococcus sp.), chrysophytes (e.g. Boekelovia sp.), haptophytes (e.g. Isochrysis sp. and Pleurochysis sp.), and bacillariophytes (e.g. Amphipleura sp., Amphora sp., Chaetoceros sp., Cyclotella sp., Cymbella sp., Fragilaria sp., Hantzschia sp., Navicula sp., Nitzschia sp., Phaeodactylum sp., and Thalassiosira sp.).

### Lipid Production

As contemplated herein, “increasing lipid production” in a cell refers to a process whereby the production of at least one type of lipid in the cell is increased compared to the production of that same lipid in the cell prior to performing the method (under the same biological conditions). It will be understood that “increasing lipid production” in a cell does not necessarily exclude decreasing the production of particular type(s) of lipid(s) in the cell, provided that the production of at least one type of lipid is increased in the cell.

Methods of the invention may be utilised to increase the production of any lipid. For example, the methods may be utilised to increase the production of any one or more of fatty acyls, glycerolipids, glycerophospholipids, cardiolipins, sphingolipids, prenols or sterols in a cell.

In some embodiments, the methods may be used to increase the production of glycerolipids in the cell. The glycerolipids may be, for example, triacyclglycerols and/or sterol esters.

The increased production of lipids in the cell may be detected using methods known in the art. Exemplary techniques include reverse-phase high performance liquid chromatography and mass spectrometry. Total lipid content in a cell may be determined, for example, using the technique of Bligh and Dyer, (see Bligh and Dyer, (1959), “A rapid method of total lipid extraction and purification”, Canadian Journal of Biochemistry and Physiology, 37(8):911-917). An exemplary method for the quantification of triacylglycerols in cellular lipid extracts is provided in Van Veldhoven et al., (1997), “Lipase-based quantitation of triacylglycerols in cellular lipid extracts: Requirement for presence of detergent and prior separation by thin-layer chromatography”, Lipids, 32(12): 1297-1300. Additional non-limiting examples of methods for quantifying lipids are provided in U.S. Pat. No. 4,370,311 (issued on 25 Jan., 1983 to Ilekis, John V) and U.S. Pat. No. 5,491,093 (issued on 13 Feb., 1996 to Yamamoto, et al.).

Methods of the invention may be used to increase the production of triacylglycerols (TAGs) in a cell. A “triacylglycerol” (also known as a “triglyceride”) as contemplated herein is a glycerolipid formed from the binding of three fatty acids to a glycerol molecule. The three fatty acids may be identical. Alternatively, two of the fatty acids may be identical. Alternatively, each fatty acid of the triacylglycerol may be a different fatty acid.

The triacylglycerol may comprise fatty acids of any length. In certain embodiments of the invention, the triacylglycerol comprises at least one long chain fatty acid having greater than 18 carbon atoms. In other embodiments of the invention, the triacylglycerol comprises at least one medium chain fatty acid having 14 carbon atoms to 18 carbon atoms. In preferred embodiments the triacylglycerol comprises at least one short chain fatty acid having less than 14 carbon atoms.

The triacylglycerol may comprise saturated and/or unsaturated fatty acids. The unsaturated fatty acid may comprise any number of double and/or triple carbon bonds. It will be understood that triacylglycerols comprising one or more unsaturated fatty acids as contemplated herein include cis and trans configurational isomers of those unsaturated fatty acid(s). The triacylglycerol may comprise an unsaturated fatty acid of any length. For example, the unsaturated fatty acid may be a short chain fatty acid, a medium chain fatty acid (e.g. 9-hexadecenoic acid (16:1); 9-octadecenoic acid (18:1), 12-hydroxy-9-octadecenoic acid (18:1), 11 octadecenoic acid (18:1), 9,12-octadecadienoic acid (18:2), 9,12,15-octadecatrienoic acid (18:3), 6,9,12-octadecatrienoic acid (18:3)), or a long chain fatty acid

(e.g. 9-eicosenoic acid (20:1), 5,8,11,14-eicosatetraenoic acid (20:4), 5,8,11,14,17-eicosapentaenoic acid (20:5), 13-docosenoic acid (22:1), 4,7,10,13,16,19-docosahexaenoic acid (22:6)).

The triacylglycerol may comprise a saturated fatty acid of any length. For example, the saturated fatty acid may be a short chain fatty acid (e.g. butanoic acid (C4:0); hexanoic acid (C6:0); octanoic acid (C8:0), decanoic acid (C10:0), dodecanoic acid (C12:0)), a medium chain fatty acid (e.g. tetradecanoic acid (C14:0); hexadecanoic acid (C16:0); octadenoic acid (C18:0)), or a long chain fatty acid (e.g. eicosanoic acid (20C); docosanoic acid (22C); tetracosanoic acid (24C)).

The production of biodiesel from triacylglycerols with shorter acyl chains may reduce the level of viscosity. Accordingly, in certain embodiments a triacylglycerol produced in accordance with the methods of the invention comprises at least one saturated medium chain fatty acid having 16 carbon atoms and/or at least one saturated medium chain fatty acid having 14 carbon atoms. More preferably, a triacylglycerol produced in accordance with the methods of the invention comprises at least one saturated short chain fatty acid having 12 carbon atoms, more preferably 10 carbon atoms, and still more preferably less than 10 carbon atoms.

### Phosphatidic Acid Production and the PEMT Pathway

Increasing cellular levels of phosphatidic acid (1,2-Diacyl-sn-glycerol 3-phosphate) is demonstrated herein to correlate with increased production of glycerolipids (e.g. triacylglycerols and/or sterol esters), and the production of glycerolipids with short and/or saturated fatty acid chains.

Accordingly, in certain aspects the invention provides methods for increasing the production of a lipid in a cell by raising phosphatidic acid production in the cell.

Phosphatidic acid production may be increased in the cell by any means.

For example, phosphatidic acid production may be increased in a cell by increasing the quantity and/or activity of one or more proteins or compounds of a lipid biosynthetic pathway of the cell. Alternatively, phosphatidic acid production may be increased in a cell by decreasing the quantity or activity of one or more proteins in a lipid biosynthetic pathway of the cell.

Non-limiting examples of such proteins include enzymes, transcription factors, regulatory proteins, intermediate proteins, and precursor proteins.

The quantity and/or activity of the protein may be increased or decreased by any means including, but not limited to, modifying the expression of a gene encoding the protein, modifying post-translational processing of the protein, agonizing a biological function of the protein, or antagonizing a biological function of the protein.

In some embodiments, phosphatidic acid production in a cell may be increased by increasing the quantity and/or activity of one or more proteins or compounds involved in a lipid biosynthetic pathway. For example, the quantity and/or activity of a protein (e.g. a precursor protein, an intermediate protein, an enzyme, a regulatory protein and/or a transcription factor) or compound in a pathway that positively regulates phosphatidic acid synthesis, either directly or indirectly, may be increased for the purpose of raising phosphatidic acid production in the cell. Non-limiting examples of proteins or compounds in this category include glycerol, glycerol-3-phosphate, glycerol kinase (see EC 23.1.30 of the Nomenclature Committee of the International Union of Biochemistry and Molecular Biology), dihydroxyacetonephosphate, glycerol-3-dehydrogenase (EC 1.1.99.5, EC 1.1.1.94, EC 1.1.1.8), acetyl-CoA precursors, 1-Acyl-sn-glycerol 3-phosphate, Glycerol-3-phosphate O-acyltransferase (EC 2.3.1.15), and 1-Acylglycerol-3-phosphate O-acyltransferase (EC 2.3.1.51). The quantity and/or activity of such proteins may be increased by increasing the expression of genes encoding them.

In other embodiments, phosphatidic acid production in a cell may be increased by reducing the quantity and/or activity of one or more proteins or compounds involved in a lipid biosynthetic pathway. For example, the quantity and/or activity of a protein (e.g. a precursor protein, an intermediate protein, an enzyme, a regulatory protein and/or a transcription factor) or compound in a pathway that negatively regulates phosphatidic acid synthesis, either directly or indirectly, may be increased for the purpose of raising phosphatidic acid production in the cell. The quantity and/or activity of such proteins may be decreased by reducing the expression of genes encoding them.

In certain embodiments, phosphatidic acid levels may be increased in the cell by inhibiting the phosphatidylethanolamine N-methyltransferase (PEMT) pathway. As is known to the skilled addressee, the PEMT pathway involves the methylation of phosphatidylethanolamine to phosphatidylcholine in three steps by two methyltransferases, phosphatidylethanolamine N-methyltransferase (EC 2.1.1.17) and phospholipid methyltransferase (EC 2.1.1.16).

In accordance with the methods of the invention, “inhibiting” the PEMT pathway in a cell encompasses any means of reducing the occurrence or rate of one or more steps in the PEMT pathway. This may be achieved by targeting one or more components of the PEMT pathway directly, for example by reducing the quantity or activity of an enzyme, enzyme cofactor, precursor compound, and/or intermediate compound in the pathway. Additionally or alternatively, “inhibiting” the PEMT pathway in a cell may be achieved indirectly, for example, by increasing or decreasing the quantity and/or activity of other biological molecules that are not components within the PEMT pathway but nonetheless exert an influence on the pathway. For example, the activity of regulatory proteins (e.g. transcription factors) having effect on the expression of PEMT pathway proteins may be modified. Additionally or alternatively, the quantity and/or activity of component(s) in other interrelated lipid biosynthetic pathways (or the operation of these pathways in general) may be altered thereby inhibiting the PEMT pathway.

The skilled addressee can readily determine whether the PEMT pathway is inhibited in a given cell or microorganism by measuring PEMT pathway activity in that cell, and comparing it to PEMT pathway activity measured in substantially similar or identical untreated/wild-type cells (i.e. control cells). Assays for measuring PEMT pathway activity are well known in the art (e.g. radio-enzymatic assays). Non-limiting examples of suitable assays include those described in Hirata and Axelrod, (1978), “Enzymatic synthesis and rapid translocation of phosphatidylcholine by two methyltransferases in erythrocyte membranes” Proc. Natl. Acad. Sci. U.S.A. 75, 2348-2352; Hirata et al., (1978), “Identification and properties of two methyltransferases in conversion of phosphatidylethanolamine to phosphatidylcholine”, Proc. Natl. Acad. Sci. U.S.A. 75, 1718-1721; Ridgway and Vance, (1992), “Phosphatidylethanolamine N-methyltransferase from rat liver”, Methods Enzymol. 209, 366-374; and Duce et al., (1988), “S-Adenosyl-L-methionine synthetase and phospholipid methyltransferase are inhibited in human cirrhosis”, Hepatology 8: 65-68.

In some embodiments of the invention, the PEMT pathway is inhibited by reducing the quantity or activity of:

(i) phospholipid methyltransferase (EC 2.1.1.16);

(ii) phosphatidylethanolamine methyltransferase (EC 2.1.1.17);

(iii) inositol-1-phosphate synthase (EC 5.5.1.4);

(iv) CDP-diacylglycerol synthase (EC: 2.7.7.41);

(v) casein kinase II (EC 2.7.11.1);

(vi) a beta regulatory subunit of casein kinase II (EC 2.7.11.1);

(vii) a transcription factor that positively regulates one or more components of the PEMT pathway—for example, Saccharomyces cerevisiae INO2/YDR123C and/or INO4/YOL108C, or a protein homologous to Saccharomyces cerevisiae INO2/YDR123C or INO4/YOL108C;

(viii) Saccharomyces cerevisiae RTC2/YBR147W, or a protein homologousto Saccharomyces cerevisiae RTC2/YBR147W;

(ix) Saccharomyces cerevisiae MRPS35/YGR165W or a protein homologous to Saccharomyces cerevisiae MRPS35/YGR165W;

(x) Saccharomyces cerevisiae FLD1/YLR404W, or a protein homologous to Saccharomyces cerevisiae FLD1/YLR404W;

(xi) any combination of two or more of (i)-(x).

The skilled addressee will understand that “homologous proteins” in this context are identical or similar proteins from a different microorganism or plant sharing substantially the same biological function or activity as the original protein referred to.

In some embodiments, the PEMT pathway is inhibited by reducing the quantity or activity of a Brassica sp. plant CDP-diacylglycerol synthase protein comprising the sequence defined by SEQ ID NO:1; a Ricinus sp. plant CDP-diacylglycerol synthase protein comprising the sequence defined by SEQ ID NO:6; a Glycine sp. plant CDP-diacylglycerol synthase protein comprising the sequence defined by any one of SEQ ID NO:18, SEQ ID NO:19, SEQ ID NO:20 or SEQ ID NO:21; or a Chlamydomonas sp. protein encoded by the gene C_{—}30067, the gene comprising the sequence set forth in SEQ ID NO: 41.

In some embodiments, the PEMT pathway is inhibited by reducing the quantity or activity of: a Brassica sp. plant casein kinase II protein comprising the sequence defined by SEQ ID NO: 2 or SEQ ID NO: 3; a Ricinus sp. plant casein kinase II protein comprising the sequence defined by SEQ ID NO: 8 or SEQ ID NO: 9; or a Glycine sp. plant casein kinase II protein comprising the sequence defined by any one of SEQ ID NO: 22, SEQ ID NO: 23, SEQ ID NO: 24, SEQ ID NO: 25, SEQ ID NO: 26, or SEQ ID NO: 27.

In some embodiments, the PEMT pathway is inhibited by reducing the quantity or activity of: a Brassica sp. plant protein comprising the sequence defined by SEQ ID NO:4; a Ricinus sp. protein comprising the sequence defined by any one or more of SEQ ID NO: 13, SEQ ID NO: 14, SEQ ID NO: 15, SEQ ID NO: 16; or a Glycine sp. protein comprising the sequence defined by any one or more of SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36.

In some embodiments, the PEMT pathway is inhibited by reducing the quantity or activity of a Ricinus sp. phosphatidylethanolamine methyltransferase protein comprising the sequence defined by SEQ ID NO: 7.

In some embodiments, the PEMT pathway is inhibited by reducing the quantity or activity of: a Ricinus sp. phospholipid methyltransferase protein comprising the sequence defined by SEQ ID NO: 12; or a Glycine sp. phospholipid methyltransferase protein comprising the sequence defined by SEQ ID NO: 28.

In some embodiments, the PEMT pathway is inhibited by reducing the quantity or activity of a transcription factor that positively regulates one or more components of the PEMT pathway from Ricinus sp., comprising the sequence defined by SEQ ID NO: 10.

In some embodiments, the PEMT pathway is inhibited by reducing the quantity or activity of a Ricinus sp. protein comprising the sequence defined by SEQ ID NO: 11.

It will be understood that “reducing” the quantity or activity of a protein as contemplated herein encompasses any reduction of the quantity or activity of the protein when compared to its normal/standard quantity or activity under any given condition including, but not limited to, complete loss of function.

In some embodiments of the invention, the PEMT pathway is inhibited by inhibiting the expression of a gene encoding a protein in a lipid biosynthetic pathway of the cell (e.g. an enzyme, transcription factor, regulatory protein, intermediate protein, or precursor protein). For example, the PEMT pathway may be inhibited by inhibiting the expression of a gene encoding any one of the proteins referred to under (i)-(xi) above, and/or a gene encoding a protein comprising a sequence defined by any one or more of SEQ ID NOs 1-41. It will be understood that “inhibiting” gene expression as contemplated herein encompasses any reduction of gene expression when compared to the normal/standard expression of the gene under any given condition, including, but not limited to, complete loss of gene expression.

### VAMP-Associated Proteins

In additional embodiments of the invention, the production of glycerolipids is increased in a cell or microorganism by reducing the quantity or activity of a vesicle-associated membrane protein-associated protein (VAMP-associated protein).

The VAMP-associated protein may be Saccharomyces cerevisiae SCS2/YER120W or a homologous protein of another microorganism or plant.

The VAMP-associated protein may comprise a sequence defined in SEQ ID NO: 5, SEQ ID NO: 17, SEQ ID NO: 37, SEQ ID NO: 38, SEQ ID NO: 39, or SEQ ID NO: 40.

The quantity or activity of the VAMP-associated protein may be reduced by inhibiting the expression of a gene encoding the VAMP. The gene may be a homologue of S. cerevisiae scs2.

### Combinations

It will be understood that the methods of inhibiting the phosphatidylethanolamine N-methyltransferase (PEMT) pathway may be achieved by:

(a) reducing the quantity or activity of any combination of two or more of the proteins set out under (i)-(x) above;
(b) reducing the quantity or activity of any combination of two or more proteins comprising a sequence defined by any one of SEQ ID NOs: 1-4, 6-16, or 18-36;
(c) reducing the quantity or activity of any combination of: at least one protein set out under (i)-(x) above and at least one protein comprising a sequence defined by any one of SEQ ID NOs: 1-4, 6-16, or 18-36;
(d) reducing the quantity or activity of any combination of: at least one protein set out under (i)-(x) above and at least one vesicle-associated membrane protein (VAMP)-associated protein including, but not limited to, at least one VAMP comprising any one of SEQ ID NO: 5, SEQ ID NO: 17, SEQ ID NO: 37, SEQ ID NO: 38, SEQ ID NO: 39, or SEQ ID NO: 40;
(e) reducing the quantity or activity of any combination of: at least one protein set out under (i)-(x) above, and at least one protein comprising a sequence defined by any one of SEQ ID NOs: 1-4, 6-16, or 18-36, and at least one vesicle-associated membrane protein (VAMP)-associated protein including, but not limited to, a VAMP-associated protein comprising any one of SEQ ID NO: 5, SEQ ID NO: 17, SEQ ID NO: 37, SEQ ID NO: 38, SEQ ID NO: 39, or SEQ ID NO: 40; SEQ ID Nos 19-21;
(f) reducing the quantity or activity of a protein homologous to Saccharomyces cerevisiae FLD1/YLR404W in combination with any one of (a)-(e) above.
(g) performing any one or more of (a)-(f) above by altering the expression of genes encoding the protein(s).

### Inhibition of Gene Expression

Inhibiting the expression of a gene in a cell in accordance with the methods of the invention can be performed using methods known in the art.

For example, the expression of a gene may be inhibited by reducing or eliminating transcription. Levels of gene transcription can be measured by any technique known in the art, including, for example, by transcription quantitative polymerase chain reaction (RT-PCR).

Additionally or alternatively, expression of a gene may be inhibited by reducing or eliminating the translation of transcribed gene product(s) into a protein. A change in the level of translated gene products can be measured using any technique capable of detecting and/or quantifying proteins. Suitable methods are known in the art, and include, for example, immunohistochemistry, SDS-PAGE, immunoassays, proteomics and the like.

The expression of a gene may be inhibited by the introduction of one or more mutations into the nucleotide sequence of the gene. Suitable mutations include, but are not limited to, missense mutations, nonsense mutations, truncation mutations, insertion mutations, deletion mutations or any combination thereof.

By way of non-limiting example only, a target gene in a cell (i.e. a gene encoding a protein of a PEMT pathway) may be inactivated by inserting exogenous genetic material into the gene and/or substituting the gene (or a portion of the gene) with exogenous genetic material.

In certain embodiments, exogenous DNA is integrated into the chromosomal DNA (i.e. the target gene) of the cell by recombination (e.g. homologous recombination or heterologous recombination). For example, homologous recombination may be used to substitute a target gene/portion of a target gene in the genome of a cell with similar DNA derived from a vector construct. Accordingly, a vector construct comprising a target gene sequence (or a portion of a target gene sequence) having one or more mutations (e.g. those defined above) may be introduced into the cell. Homologous recombination may facilitate replacement of the target gene (or portion of the target gene) in the host cell genome with similar sequence (comprising mutation(s)) derived from the vector construct. Exogenous genetic material incorporated into the target cell genome by homologous recombination inhibits expression of the target gene in the cell. It will be understood that a “target gene” includes regulatory sequences of that gene. Hence, the exogenous genetic material may be inserted into exonic/intronic sequences and/or regulatory sequences of the gene (e.g. promoter sequences and the like).

Cells in which the target gene is inactivated may be selected and propagated using techniques known in the art. For example, the genetic material incorporated may comprise a selectable marker (e.g. an antibiotic resistance gene) that facilitates target gene inactivation (by disrupting the coding sequence of the gene) and also allows the selection of cells in which the gene has been inactivated in the presence of an antibiotic.

Methods for the production of vector constructs suitable for introducing exogenous genetic material into a cell are generally known in the art and are described, for example, in Ausubel et al. (Eds), (2007), “Current Protocols in Molecular Biology”, John Wiley & Sons; Sambrook et al., (2001), “Molecular Cloning: A Laboratory Manual”, 3rd Ed., Cold Spring Harbor Laboratory Press; and Coico et al. (Eds), (2007), “Current Protocols in Microbiology”, John Wiley and Sons, Inc. The vector may be a plasmid vector, a viral vector, a phosmid, a cosmid or any other suitable vector construct. The vector may comprise expression control and processing sequences such as promoters, enhancers, polyadenylation signals and/or transcription termination sequences. The vector construct may also include a selectable marker, for example, an antibiotic-resistance gene such as chloramphenicol or tetracycline.

Genetic material for insertion into the construct (e.g. DNA with homology to a target gene comprising mutation(s)) may be generated, for example, by chemical synthesis techniques such as the phosphodiester and phosphotriester methods (see for example Narang et al., (1979), “Improved phosphotriester method for the synthesis of gene fragments”, Meth. Enzymol. 68:90; Brown et al., (1979), “Chemical synthesis and cloning of a tyrosine tRNA gene”, Meth. Enzymol. 68:109; and U.S. Pat. No. 4,356,270), or the diethylphosphoramidite method (see Beaucage et al., (1981), “Deoxynucleoside Phosphoramidites—A New Class of Key Intermediates for Deoxypolynucleotide Synthesis”, Tetrahedron Letters, 22:1859-1862). Genetic material for insertion into the construct may be amplified in number, for example, by performing polymerase chain reaction (PCR) assays on DNA or cDNA sequences, or by performing RT-PCR on RNA sequences. The resulting nucleic acids may then be inserted into the construct, for example, by restriction-ligation reactions or by the TA cloning method.

Suitable methods for the introduction of vector constructs and other foreign nucleic acid material into cells are generally known in the art, and are described, for example, in Ausubel et al. (Eds), (2007), “Current Protocols in Molecular Biology”, New York: John Wiley & Sons; and Sambrook et al., (2001), 3rd Ed., “Molecular Cloning: A Laboratory Manual”, Cold Spring Harbor Laboratory Press, Cold Spring Harbor, N.Y.

By way of example only, target cells may be transfected with vector constructs using the “heat shock” method. Under this method the cells are chilled in the presence of divalent cations such as Ca²⁺, which causes cell wall/membrane permeability. Cells are mixed with the construct, incubated on ice, and then briefly heat shocked (e.g. at 42° C. for 0.5-2 minutes) allowing the vector construct to enter the cell.

Alternatively, target cells may be transfected with vector constructs by electroporation, a method that involves briefly shocking the cells with an electric field causing the cells to briefly develop holes through which the construct may enter the cell. Natural membrane-repair mechanisms rapidly close these holes after the shock.

Vector constructs may be introduced into fungal cells (e.g. protoplast cells) using methods described in Pentillä et al., (1987), “A versatile transformation system for the cellulolytic filamentous fungus Trichoderma reesei”, Gene, 61:155-164.

Following entry of the vector construct into a target cell, the cell may be cultured under conditions suitable to facilitate reproduction. Reproduction of the cell facilitates replication of genetic material including that of the construct. Reproduction of the cell may also facilitate recombination between genetic material in the construct and genetic material of the host cell genome. Methods for the culture of cells are known in the art and described in, for example, Coico, et al. (Eds), (2007), “Current Protocols in Microbiology”, John Wiley & Sons, Inc.

The culture may be performed in medium containing a substrate that facilitates the identification of cells comprising vector constructs and/or cells in which a target gene has been inactivated. Accordingly, target cells containing vector constructs and/or inactivated target gene(s) may be selected and propagated. For example, if the vector contains one or more selectable markers, cells containing vector constructs may be identified by expression of the marker or markers in the presence of the relevant substrate. Using the example of a drug resistance gene such as an antibiotic resistance gene, cells containing vector constructs can be identified by their ability to grow and propagate in selection medium containing the corresponding antibiotic.

Additional sequences capable of enhancing the frequency of homologous recombination events may be included in the vector constructs. For example, eukaryotic or prokaryotic recombination enzymes such as REC A, topoisomerase, REC 1 or other DNA sequences which enhance recombination (e.g. Chi sequence) may be included in the constructs. Furthermore, sequences that enhance transcription of chimeric genes produced by homologous recombination may also be included in the vector constructs, non-limiting examples of which include inducible elements such as the metallothionine promoter. The cell may also be administered various other proteins known in the art to be capable of increasing recombination frequencies.

In certain embodiments, the expression of a gene in a cell is inhibited using an anti-sense nucleic acid to block the translation of polypeptides from RNA transcripts.

Anti-sense nucleic acids of the invention may be of at least five nucleotides in length and are generally oligonucleotides which range in length from 5 to about 200 nucleotides. For example, an anti-sense oligonucleotide of the invention may be at least about 10, 15, 20, 25, 30, 35, 40, 45, 50, 60, 70, 80, 90, 100, 125, 150, 175 or 200 nucleotides. The oligonucleotides may be DNA or RNA or chimeric mixtures or derivatives or modified versions thereof. The oligonucleotides may be single-stranded or double-stranded. In certain embodiments, the oligonucleotides are small interfering RNA (siRNA) molecules.

An anti-sense nucleic acid of the invention may be modified at any position on its structure using substituents generally known in the art. For example, the anti-sense nucleic acid may include at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, 2,2-dimethylguanine, 2-methyl-adenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, pseudouracil, 2-thiocytosine, 5-methyl-2-thiouracil, 2-thiouracil, 4-thiouracil, 5-methyluracil, uracil-5-oxyacetic acid methylester, uracil-5-oxyacetic acid (v), queosine, wybutoxosine, 5-methyl-2-thiouracil, 3-(3-amino-3-N-2-carboxypropyl) uracil, and 2,6-diaminopurine.

Anti-sense oligonucleotides, typically of 18-30 nucleotides in length (although longer or shorter length oligonucleotides are also contemplated) may be generated which are at least substantially complementary across their length to a region of the nucleic acid sequence of interest. Binding of the anti-sense oligonucleotide to a cellular nucleic acid comprising a complementary sequence may interfere with transcription, RNA processing, transport, translation and/or mRNA stability. Suitable anti-sense oligonucleotides may be prepared by methods well known to those of skill in the art, and may be designed to target and bind to regulatory regions of the target nucleotide sequence, or, to coding (gene) or non-coding (intergenic region) sequences. Suitable anti-sense oligonucleotides may include modifications designed to improve their delivery into cells, their stability once inside a cell, and/or their binding to the appropriate target. For example, the anti-sense oligonucleotide may be modified by the addition of one or more phosphorothioate linkages, or the inclusion of one or morpholine rings into the backbone (so-called ‘morpholino’ oligonucleotides).

Double-stranded RNA (dsRNA) molecules may be synthesised in vitro in which one strand is identical to a specific region of the gene transcript of interest and then introduced into the target cell.

Additionally or alternatively, corresponding dsDNA can be employed which is converted into dsRNA once presented intracellularly.

Anti-sense nucleic acids capable of inhibiting the expression of a target gene may be introduced into a cell using a replicable vector. The vector may remain episomal or integrate into the genome of the cell. By way of example only, double-stranded RNA expressing constructs may be introduced into a cell to interfere with accumulation of endogenous mRNA encoding the target gene.

Methods of synthesizing RNA molecules are known in the art and are described, for example, in Verma and Eckstein, (1998), “Modified oligonucleotides: synthesis and strategy for users”, Annu Rev Biochem., 67:99-134. Single-stranded RNAs for annealing into dsRNA molecules can also be prepared by enzymatic transcription from DNA plasmids isolated from recombinant bacteria or from synthetic DNA templates.

In certain embodiments, RNA interference (RNAi) may be used to inhibit the expression of a target gene in a cell. The RNAi may be a hairpin RNAi. RNAi refers to a means of selective post-transcriptional gene silencing by destruction of specific mRNA by small interfering RNA molecules (siRNA). The siRNA is generated by cleavage of double stranded RNA, where one strand is identical to the message to be inactivated.

The strand capable of hybridising to a portion of an RNA precursor encoding a polypeptide of the invention will, in general, have sufficient sequence complementarity to the RNA precursor to mediate target-specific RNA interference (RNAi). Accordingly, the second (non-hybridising) strand of the dsRNA molecule will have at least about 50%, 60%, 70%, 75%, 80%, 85%, or 95% sequence identity to an RNA precursor encoding a target protein. Preferably, the sequence identity is at least 85% and most preferably 100%.

In applications involving RNA interference, the length of a dsRNA molecule provided herein may be 19-25 nucleotides in length, and more preferably 20-22 nucleotides in length. In certain embodiments, at least one strand has a 3′-overhang of 1-5 nucleotides, more preferably 1-3 nucleotides and most preferably 2 nucleotides. The second strand may be blunt-ended or have up to 6 nucleotides 3′ overhang.

RNAi techniques and methods for the synthesis of suitable molecules for use in RNAi and for achieving post-transcriptional gene silencing are known in the art (see, for example, Chuang et al., (2000), Proc Natl Acad Sci USA 97: 4985-4990; Fire et al., (1998), Nature 391: 806-811; Hammond et al., (2001), Nature Rev, Genet. 2: 110-1119; Hammond et al., (2000), Nature, 404: 293-296; Bernstein et al., (2001), Nature, 409: 363-366; Elbashir et al., (2001), Nature, 411: 494-498; PCT publication no. WO 1999/32619; PCT publication no. WO 1999/49029; PCT publication no. WO 2001/29058; and PCT publication no. WO 2001/70949).

In accordance with the methods of the invention, anti-sense nucleic acids capable of inhibiting the expression of a target gene may be stably introduced and expressed in a cell (e.g. a plant cell or an algal cell) using a vector construct.

The vector may be a plasmid vector, a viral vector, a phosmid, a cosmid or any other vector construct suitable for the insertion of foreign sequences, introduction into cells and subsequent expression of the introduced sequences. In a preferred embodiment, the vector is an expression vector comprising expression control and processing sequences such as a promoter, an enhancer, polyadenylation signals and/or transcription termination sequences.

Methods for the production of vector constructs, the production of genetic material to include in vector constructs and the introduction of vector constructs into cells are generally known in the art and are described in the preceding paragraphs of this section.

A further means of inhibiting the expression of a target gene in a cell includes introducing catalytic anti-sense nucleic acid constructs, such as ribozymes, which are capable of cleaving mRNA transcripts and thereby preventing the production of wild type protein. Ribozymes are targeted to and anneal with a particular sequence by virtue of two regions of sequence complementarity to the target flanking the ribozyme catalytic site. After binding the ribozyme cleaves the target in a site-specific manner. The design and testing of ribozymes which specifically recognise and cleave sequences of interest can be achieved using techniques well known to those in the art (see, for example, Lieber and Strauss, (1995), “Molecular and Cellular Biology”, 15:540-551).

### Reducing Protein Activity

Reducing the activity of a protein in a cell in accordance with the methods of the invention can be performed using methods known in the art.

It will be understood that “reducing” protein activity as contemplated herein encompasses any reduction of the activity of the protein compared to its normal/standard activity at any given condition including, but not limited to, complete loss of activity.

By way of non-limiting example only, the activity of a target protein (e.g. a PEMT pathway protein) may be reduced by administering an antagonist of the protein to the cell. The antagonist may inhibit one or more of the biological activities of the protein, for example, by preventing interaction(s) with other protein(s) in the cell.

Antagonists of target proteins may be generated using a number of techniques known to those skilled in the art. For example, methods such as X-ray crystallography and nuclear magnetic resonance spectroscopy may be used to model the structure of a target protein, thus facilitating the design of potential modulating agents using computer-based modelling. Various forms of combinatorial chemistry may also be used to generate putative antagonists.

An antagonist of a target protein can potentially be any chemical compound, non-limiting examples of which include amino acids, nucleic acids, peptide nucleic acids, lipids, polypeptides, carbohydrates, and nucleosides. Other non-limiting examples include peptidomimetics (e.g. peptoids), amino acid analogues, polynucleotides, polynucleotide analogues, nucleotides, nucleotide analogues, metabolites, metabolic analogues, and organic or inorganic compounds (including heteroorganic and organometallic compounds).

Large libraries of chemicals (i.e. putative antagonists) may be screened for antagonistic activity against a target protein or target protein(s) using high-throughput methods. Such libraries of candidate compounds can be generated or purchased from commercial sources. For example, a library can include 10,000, 50,000, or 100,000 or more unique compounds. By way of example only, a library may be constructed from heterocycles including benzimidazoles, benzothiazoles, benzoxazoles, furans, imidazoles, indoles, morpholines, naphthalenes, piperidines, pyrazoles, pyridines, pyrimidines, pyrrolidines, pyrroles, quinolines, thiazoles, thiphenes, and triazines. A library may comprise one or more classes of chemicals, for example, those described in Carrell et al., (1994), Angew. Chem. Int. Ed. Engl. 33:2059; Carell et al., (1994), Angew. Chem. Int. Ed. Engl. 33:2061; Cho et al., (1993), Science 261:1303-1305; DeWitt et al., (1993), Proc. Natl. Acad. Sci. U.S.A. 90:6909-6913; Erb et al., (1994), Proc. Natl. Acad. Sci. USA 91:11422-11426; Gallop et al., (1994), J. Med. Chem. 37:1233-1251; and Zuckermann et al., (1994), J. Med. Chem. 37:2678-2685.

In general, putative antagonists may be screened for antagonistic activity against target protein(s) by contacting the putative antagonist with the protein(s) under conditions suitable for an interaction to occur.

Putative antagonists which bind, or otherwise interact with target protein(s) (or nucleic acids encoding a target protein), and specifically agents which inhibit their activity, may be identified by a variety of suitable methods. Non-limiting methods include the two-hybrid method, co-immunoprecipitation, affinity purification, mass spectroscopy, tandem affinity purification, phage display, label transfer, DNA microarrays/gene coexpression and protein microarrays.

In certain embodiments of the invention, the activity of a target protein in a cell may be inhibited by an antibody specific for the protein.

An antibody that “specific for” a target protein is one capable of binding to the target protein with a significantly higher affinity than it binds to an unrelated molecule (i.e. a non-target protein). Accordingly, an antibody specific for a target protein is an antibody with the capacity to discriminate between the target protein and any other number of potential alternative binding partners. Hence, when exposed to a plurality of different but equally accessible molecules as potential binding partners, an antibody specific for a target protein will selectively bind to the target protein and other alternative potential binding partners will remain substantially unbound by the antibody. In general, an antibody specific a target protein will preferentially bind to the target protein at least 10-fold, preferably 50-fold, more preferably 100-fold, and most preferably greater than 100-fold more frequently than other potential binding partners that are not the target protein. An antibody specific for a target protein may be capable of binding to other non-target molecules at a weak, yet detectable level. This is commonly known as background binding and is readily discernible from target protein-specific binding, for example, by use of an appropriate control.

Preferably suitable antibodies are prepared from discrete regions or fragments of the target protein. An antigenic portion of a target protein may be of any appropriate length, such as from about 5 to about 15 amino acids. Preferably, an antigenic portion contains at least about 5, 6, 7, 8, 9, 10, 11, 12, 13 or 14 amino acid residues.

An antibody that binds specifically to a target protein can be generated using methods known in the art. For example, a monoclonal antibody specific for target protein, typically containing Fab portions, may be prepared using the hybridoma technology described in Harlow and Lane (eds.), (1988), “Antibodies-A Laboratory Manual”, Cold Spring Harbor Laboratory, N.Y. In essence, in the preparation of monoclonal antibodies directed toward a target protein, any technique that provides for the production of antibodies by continuous cell lines in culture may be used. These include the hybridoma technique originally developed by Kohler and colleagues (see Kohler et al., (1975), “Continuous cultures of fused cells secreting antibody of predefined specificity”, Nature, 256:495-497) as well as the trioma technique.

Screening for the desired antibody can also be accomplished by a variety of techniques known in the art. Suitable assays for immunospecific binding of antibodies include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, immunoelectrophoresis assays, and the like (see, for example, Ausubel et al., (1994), “Current Protocols in Molecular Biology”, Vol. 1, John Wiley & Sons, Inc., New York).

In certain embodiments, a vector construct capable of expressing an antagonist of a target protein may be used to reduce the activity of the target protein in the cell. Methods for generating vector constructs and administering them to cells are known in the art described in the section above entitled “Inhibition of gene expression”.

For example, a vector construct capable of expressing an antibody or antibody fragment specific for a target protein may be administered to the cell. Methods for the generation of such constructs and their expression in target cells are known in the art and exemplary methods are provided, for example, in U.S. Pat. No. 7,112,439 (issued on 26 Sep., 2006 to Johnson et al); US patent publication no. 2009-0104660 (filed on 23 Apr., 2009, Yung et al.); Cabilly et al., (1984), “Generation of antibody activity from immunoglobulin polypeptide chains produced in Escherichia coli”, Proc. Natl. Acad. Sci. USA 81: 3273-3277, and Boss et al., (1984), “Assembly of functional antibodies from immunoglobulin heavy and light chains synthesised in E. coli”, Nucleic Acids Res. 12: 3791-3806.

### Energy Storage

In accordance with the methods of the invention, increasing levels of phosphatidic acid in a cell may provide a means of generating large/supersized lipid droplets within the cell. Without limitation to a particular mode of action, it is postulated that increasing cellular levels of phosphatidic acid serves to enhance the fusion properties of intracellular lipid droplets resulting in the formation of large/supersized lipid droplets within the cell.

Fusing multiple lipid droplets with a cell to form large lipid droplet(s) may provide a means of modifying lipid storage within the cell. In particular, fusing multiple lipid droplets with a cell to form large lipid droplet(s) may be utilised as a means to improve storage of energy within the cell.

In certain embodiments, the cell may be a microorganism or plant, non-limiting examples of which are provided in the section above entitled “Microorganisms and plants”.

The levels of phosphatidic acid may be increased in the cell using any suitable method, including but not limited to those described in the section above entitled “Phosphatidic acid production and the PEMT pathway”.

### Genetically Modified Microorganisms and Plants

Certain aspects the invention relate to genetically modified organisms (e.g. microorganisms and plants) in which the production of a lipid (e.g. a glycerolipid) is increased compared to a corresponding wild-type organism. In general, the genetically modified organisms comprise at least one genetic modification that increases phosphatidic acid production.

The “genetic modification” may be any change to the genetic make-up of the organism, non-limiting examples of which include the introduction of mutation(s) into endogenous genetic material, the deletion of endogenous genetic material, and the introduction of exogenous genetic material (e.g. exogenous genetic material integrated into the genome of the cell; exogenous genetic material introduced into the cell by way of a construct such as a plasmid or virus; exogenous nucleic acids such as oligonucleotides, RNA molecules (including RNAi) and the like).

The genetic modification may be a permanent genetic modification (i.e. one that is stably inherited by progeny) or a transient genetic modification (i.e. one that is lost after one or more generations of progeny).

It will be understood that the genetically modified organisms of the present invention are non-human organisms.

A genetically modified organism in accordance with the invention may be any organism capable of lipid production, in particular glycerolipid production, and preferably triacylglycerol and/or sterol ester production.

For example, the modified microorganism may be a bacterial, algal, or fungal species, non-limiting examples of which are provided above in the section entitled “Microorganisms and plants”.

A genetically modified plant in accordance with the invention may be any plant capable of lipid production and in particular glycerolipid production. Non-limiting examples of such plants are provided above in the section entitled “Microorganisms and plants”.

Genetically modified organisms of the invention are capable of increased lipid production. It will be understood that “increased lipid production” in a genetically modified organism of the invention refers to the increased production of at least one type of lipid in the organism compared to the production of that same lipid (under the same biological conditions) in a corresponding organism that has not been genetically modified. This does not necessarily exclude decreased production of particular type(s) of lipid(s) in the genetically modified organism provided that the production of at least one type of lipid is increased.

Preferably, the production of triacylglycerols and/or sterol esters is increased in genetically modified microorganisms and plants of the invention compared to a corresponding wild-type microorganism or plant. Preferably, the triacylglycerols and/or sterol esters comprise short and/or saturated acyl chains. Non-limiting examples of preferred triacylglycerols and preferred fatty acid chain components of the triacylglycerols and sterol esters are provided in the section above entitled “Lipid production”.

Genetically modified organisms of the invention may comprise any genetic modification that increases cellular production of phosphatidic acid. For example, the modification may inhibit the phosphatidylethanolamine N-methyltransferase (PEMT) pathway. It will be understood that a “genetic modification” as contemplated herein includes, but is not limited to, any modification to genetic material of the organism (e.g. DNA, RNA, cDNA and the like) and the introduction/expression of foreign genetic material in cell(s) of the organism. The invention also contemplates genetically modified organisms generated by selective breeding. Preferably, the genetic modification is of a permanent or semi-permanent nature such that it is inherited by offspring of the organism. Preferably, the genetic modification is of a permanent nature such that it is inherited by offspring of the microorganism or plant. It will also be recognised that although the modification may be introduced by direct genetic manipulation of a target gene in cells of the organism, selection and classical genetics involving sexual crosses may also be utilised in appropriate cases.

The invention provides modified microorganisms and plants having increased levels of phosphatidic acid production in their cell(s). Phosphatidic acid production may be increased in cell(s) of the microorganisms and plants by any suitable genetic modification.

For example, phosphatidic acid production may be increased by introducing a genetic modification that increases the quantity and/or activity of one or more proteins or compounds in a lipid biosynthetic pathway. Alternatively, phosphatidic acid production may be increased by introducing a genetic modification that decreases the quantity or activity of one or more proteins or compounds in a lipid biosynthetic pathway. Non-limiting examples of genes and combinations of genes that may be subjected to modifications to increase phosphatidic acid production are provided in the section above entitled “Phosphatidic acid production and the PEMT pathway”.

In certain embodiments, the genetic modification inhibits the phosphatidylethanolamine N-methyltransferase (PEMT) pathway. Any genetic modification that inhibits the PEMT pathway may be utilised. Non-limiting examples of genes and combinations of genes that may be subjected to modification so as to increase phosphatidic acid production and/or inhibit the phosphatidylethanolamine N-methyltransferase (PEMT) pathway are provided in the section above entitled “Phosphatidic acid production and the PEMT pathway”.

In some embodiments of the invention, the PEMT pathway is inhibited in the modified microorganism or plant by introducing one or more genetic modification that reduces the expression of a gene encoding:

(i) phospholipid methyltransferase (EC 2.1.1.16);

(ii) phosphatidylethanolamine methyltransferase (EC 2.1.1.17);

(iii) inositol-1-phosphate synthase (EC 5.5.1.4);

(iv) CDP-diacylglycerol synthase (EC: 2.7.7.41);

(v) casein kinase II (EC 2.7.11.1);

(vi) a beta regulatory subunit of casein kinase II (EC 2.7.11.1);

(vii) a transcription factor that positively regulates one or more components of the PEMT pathway—for example, Saccharomyces cerevisiae INO2/YDR123C and/or INO4/YOL108C, or a protein homologous to Saccharomyces cerevisiae INO2/YDR123C or INO4/YOL108C;

(viii) Saccharomyces cerevisiae RTC2/YBR147W, or a protein homologous to Saccharomyces cerevisiae RTC2/YBR147W;

(ix) Saccharomyces cerevisiae MRPS35/YGR165W or a protein homologous to Saccharomyces cerevisiae MRPS35/YGR165W;

(x) Saccharomyces cerevisiae FLD1/YLR404W, or a protein homologous to Saccharomyces cerevisiae FLD1/YLR404W;

(xi) a Brassica sp. plant CDP-diacylglycerol synthase protein comprising the sequence defined by SEQ ID NO:1; a Ricinus sp. plant CDP-diacylglycerol synthase protein comprising the sequence defined by SEQ ID NO:6; a Glycine sp. plant CDP-diacylglycerol synthase protein comprising the sequence defined by any one of SEQ ID NO:18, SEQ ID NO:19, SEQ ID NO:20 or SEQ ID NO:21; or a Chlamydomonas sp. protein encoded by the gene C_{—}30067, the gene comprising the sequence set forth in SEQ ID NO: 41;

(xii) a Brassica sp. plant casein kinase II protein comprising the sequence defined by SEQ ID NO: 2 or SEQ ID NO: 3; a Ricinus sp. plant casein kinase II protein comprising the sequence defined by SEQ ID NO: 8 or SEQ ID NO: 9; or a Glycine sp. plant casein kinase II protein comprising the sequence defined by any one of SEQ ID NO: 22, SEQ ID NO: 23, SEQ ID NO: 24, SEQ ID NO: 25, SEQ ID NO: 26, or SEQ ID NO: 27;

(xiii) a Brassica sp. plant protein comprising the sequence defined by SEQ ID NO:4; a Ricinus sp. protein comprising the sequence defined by any one or more of SEQ ID NO: 13, SEQ ID NO: 14, SEQ ID NO: 15, SEQ ID NO: 16; or a Glycine sp. protein comprising the sequence defined by any one or more of SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36;

(xiv) a Ricinus sp. phosphatidylethanolamine methyltransferase protein comprising the sequence defined by SEQ ID NO: 7;

(xv) a Ricinus sp. phospholipid methyltransferase protein comprising the sequence defined by SEQ ID NO: 12; or a Glycine sp. phospholipid methyltransferase protein comprising the sequence defined by SEQ ID NO: 28.

(xvi) a transcription factor that positively regulates one or more components of the PEMT pathway from Ricinus sp., comprising the sequence defined by SEQ ID NO: 10;

(xvii) a Ricinus sp. protein comprising the sequence defined by SEQ ID NO: 11;

(xviii) any combination of two or more of (i)-(xvii)

In certain embodiments, the genetic modification inhibits a vesicle-associated membrane protein-associated protein (VAMP-associated protein).

Non-limiting examples of VAMP-associated proteins that may be inhibited include Saccharomyces cerevisiae SCS2/YER120W or a homologous protein of another microorganism or plant; or a VAMP-associated protein comprising a sequence defined in SEQ ID NO: 5, SEQ ID NO: 17, SEQ ID NO: 37, SEQ ID NO: 38, SEQ ID NO: 39, or SEQ ID NO: 40.

Genetically modified organisms of the invention may be generated using standard methods known in the art.

For example, vector construct(s) encoding anti-sense nucleic acid(s) capable of inhibiting expression of a target gene may be introduced and expressed in cell(s) of the organism. Suitable anti-sense nucleic acids, vector constructs, and methods for their administration to cells are described above in the section entitled “Inhibition of gene expression”. Alternatively, the vector construct may encode an antagonist of a target protein. Methods for the generation of suitable antagonists and their introduction into cells are described above in the section entitled “Reducing protein activity”.

In certain embodiments, genetically modified organisms of the invention comprise one or more mutations in a gene encoding a protein of a starch biosynthetic pathway or a polyhydroxyalkanoate biosynthetic pathway. Suitable mutations include, but are not limited to, missense mutations, nonsense mutations, truncation mutations, insertion mutations, deletion mutations or any combination thereof. Mutation(s) may be introduced into a gene of an organism using any method known in the art. For example, mutation(s) may be introduced by administering a vector construct to cell(s) of the organism facilitating the exchange of host genetic material with exogenous genetic material of the vector using methods described in the section above entitled “Inhibition of gene expression”.

In certain embodiments of the invention, genetically modified organisms are generated by introduction of a suicide vector into cell(s) of the organism. As contemplated herein, a suicide vector is a vector that is unable to replicate in a particular host and is maintained only if it recombines into the host genome. The suicide vector may comprise sequences that are homologous to DNA sequences flanking the target gene, homologous to DNA sequences within the target gene, or both. In general, the target gene encodes a protein (e.g. an enzyme or transcription factor) of a starch or polyhydroxyalkanoate biosynthetic pathway, suitable examples of which are provided in the section above entitled “Phosphatidic acid production and the PEMT pathway”.

Methods for the production of suicide vectors and their introduction into cells are well known in the art and are described, for example, in Ausubel, et al. (Eds), (2007), “Current Protocols in Molecular Biology”, New York: John Wiley & Sons; and, Sambrook et al., (2001), 3rd Ed., “Molecular Cloning: A Laboratory Manual”, Cold Spring Harbor Laboratory Press, Cold Spring Harbor, N.Y. Genetic material for insertion into the suicide construct may be generated, for example, using the methods described in the section above entitled “Inhibition of gene expression”.

Cell(s) in which exogenous genetic material from the vector has been incorporated into genomic DNA may be selected and propagated. In the case where the cell is derived from a multicellular organism (e.g. a plant) a genetically modified organism may be generated from the cell using techniques known in the art.

The skilled addressee will recognise that the methods described above for inhibiting gene expression or inhibiting protein function in genetically modified microorganisms and plants of the invention are non-limiting examples, and that other suitable methods known in the art may also be utilised.

### Biofuel Production

Additional aspects of the invention relate to the production of biofuels from lipids. In certain embodiments, the biofuel is biodiesel. The lipids may be produced by a cell in accordance with the methods described herein. Alternatively, the lipids may be produced by a genetically modified organism (e.g. microorganism or plant) of the invention.

In general, the production of lipids from a genetically modified organism of the invention may be facilitated by cultivating the organisms under conditions suitable for their growth and/or reproduction. It will be understood that the term “cultivating” as contemplated herein encompasses any method of promoting the growth and/or, reproduction of an organism (e.g. algae, plants, bacteria, fungi). Methods for cultivating organisms such as algae, plants, bacteria, fungi are well known in the art.

Algae such as microalgae may be cultivated, for example, in closed or open ponds or photobioreactors. The growth rate of algae during cultivation may be regulated by varying factors including, but not limited to, light, temperature, salinity/pH of liquid medium, aeration (e.g. nitrogen and CO₂ exposure), photoperiod (i.e. light and dark cycles), and availability of nutrients (e.g. Ca(NO₃)₂; KH₂PO₄; MgSO₄; NaHCO₃, NaNO₃, MgSO₄, NaCl; K₂HPO₄; KH₂PO₄, CaCl₂, Na₂HPO₄, trace elements/metals).

Preferably, the lipid is a glycerolipid. More preferably, the lipid is a triacylglycerol. Non-limiting examples of preferred triacylglycerols and preferred fatty acid chain components of the triacylglycerols are provided in the section above entitled “Lipid production”. In addition to triacylglycerols, lipids produced by a cell in accordance with the methods described herein and/or a genetically modified microorganism or plant of the invention may include, for example, free oils, fats, greases, diacylglycerides, and/or phospholipids.

In general, biofuel production methods of the invention comprise the step of isolating lipids from cells in which they are produced. Techniques for the extraction of lipids from cells (e.g. plant, algal and bacterial cells) are well known in the art. For examples, mechanical crushing (e.g. using an expeller or press) followed by chemical extraction using solvents such as aliphatics, supercritical liquids and gases, hexane, acetone, and primary alcohols, may be used to extract lipids from cells.

The methods of lipid extraction may incorporate additional biological agents to enhance the separation of materials and/or improve reaction rates. For example, various agents may be used to depolymerise algal/plant cell walls to assist in releasing lipids. Additionally or alternatively, viruses specific to specific algae (e.g. Chlorella Virus) and/or cyanophages (e.g. SM-1, P-60, and AS-1) may be utilised to rupture algal cell walls.

Algal and/or plant matter remaining after lipid extraction may be used as a source of biomass for alternative biofuel production processes (e.g. generation of bioalcohols via the separation and hydrolysis of starch followed by fermentation of short-chain sugars).

Lipids extracted in accordance with the invention may be treated using oxidative processes known in the art (e.g. ozonation, peroxone oxidation) to alter the degree of saturation of the lipids. This may increase the market value of the extracted lipids for bio fuel production.

Extracted lipids can be further processed to produce biofuels.

In preferred embodiments, biodiesel is generated from extracted lipids. For example, acid esterification, base transesterification, and combinations thereof may be used to generate biodiesels from triacylglycerols. Transesterification is a process which includes reacting the triacylglycerol with alcohol or another alternative acyl acceptor to produce free fatty acid esters and glycerol.

Biodiesel may be produced via homogeneous base, acid, and/or enzyme catalyzed transesterification, as well as heterogeneous catalysed processes.

Base-catalysed transesterification may be used to transform triacylglycerols into alkyl esters (i.e. biodiesel). For example, an alcohol (e.g. methanol, ethanol, iso-propanol) mixed with a base such as potassium hydroxide or sodium hydroxide may be used to produce biodiesel from triacylglycerols using base-catalysed transesterification.

Additionally or alternatively, acid-catalysed transesterification may be used to transform triacylglycerols into biodiesel. For example, an alcohol (e.g. methanol, ethanol, iso-propanol) mixed with an acid (e.g. sulfuric acid) may be used to produce biodiesel from triacylglycerols using acid-catalysed transesterification. The rate of acid-catalysed transesterification may be enhanced by catalysing the reaction at increased temperature and/or pressure (e.g. more than 60° C. and/or more than 3 atmospheres)

Additionally or alternatively, triacylglycerols may be converted to biodiesel by direct hydrogenation.

In addition to the use of extracted triacylglycerols for biofuel production, extracted lipids (e.g. free oils, fats, greases, diacylglycerides and/or phospholipids) may be added directly to petroleum diesel as a blending agent.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

The invention will now be described with reference to specific examples, which should not be construed as in any way limiting.

### Example 1

### Role for Phosphatidic Acid in the Formation of “Supersized” Lipid Droplets

### Materials and Methods

### (i) Yeast Strains

S. cerevisiae wild type strain BY4741 (MATa; his3Δ1; leu2Δ0; met15Δ0; ura3Δ0) and its derived non-essential gene-deletion strains were either obtained from EUROSCARF or generated in this study. The latter included pah1Δ (PAH1::HIS3MX6), and dga1Δlro1Δ (DGA1::kanMX4, LRO1::hphNT1). The Tet-promoter strains used for expression of essential genes under the regulatable TetO7 promoter were obtained from Open biosystems.

### (ii) Reagents

Yeast extract, peptone, dextrose, and yeast nitrogen base were purchased from BD. Nile red, choline, ethanolamine, inositol, doxycycline, cerulenin, and Ficoll 400 were from Sigma. 1, 2-dioctanoyl-sn-glycerol 3-phosphate (PA 8:0/8:0), 1,2-dioleoyl-sn-glycero 3-phosphate (PA 18:1/18:1), oleoyl-L-α-lysophosphatidic acid, 1,2-dioctanoyl-sn-glycerol (DAG 8:0/8:0), phosphatidylethanolamine, phosphatidylcholine, and triolein were purchased from Avanti Polar Lipids.

To screen for yeast mutants that generate supersized LDs, cells were cultured in synthetic complete media (0.67% yeast nitrogen base, 2% dextrose, and amino acids) in 96-well plates at 30° C. till stationary phase. For TetO7-regulated strains, 15 μg/ml doxycycline was added to repress specific genes. Cells were stained with 20 μg/ml Nile red for LDs and observed by fluorescence microscopy. Yeast strains found to contain SLDs were recultured in synthetic complete media with aeration in 10 ml culture tubes to confirm the phenotype. These strains were also grown in YPD media (1% yeast extract, 2% peptone, and 2% dextrose) to examine the morphology of LDs.

For phospholipid precursor treatment, synthetic complete medium was supplemented with 1 mM choline, 1 mM ethanolamine, or 75 μM inositol.

(iii) Fluorescence Microscopy

Fluorescent imaging was performed under a Leica CTR5500 microscope (Wetzlar, Germany) with an EL6000 fluorescent lamp. Images were taken with a DFC300 FX digital camera and a Leica LAS AF software. Yeast cells were viewed under a ×100/1.30 oil immersion objective lens. A 450-490-nm bandpass excitation filter, a 510 dichromatic mirror, and a 515-nm longpass emission filter (Leica filter cube I3) were chosen to observe Nile red-stained LDs. For statistical presentation of the percentage of cells containing supersized LDs, 200 cells were observed and percentage was calculated. The experiments were done in triplicates and the result was shown as mean±SD. Mammalian LDs were stained with Bodipy 493/503 (Invitrogen) and observed with a 470/40-nm bandpass excitation filter, a 500-nm dichromatic mirror, and a 525/50-nm bandpass emission filter (Leica filter cube GFP).

To observe and record LD fusion, 3 μl of mid-log phase cells (OD₆₀₀˜1.5) or purified LDs were stained with Nile red, spotted on a slide and covered with a coverslip. Under the microscope, cells in which two or several LDs lay close together were targeted. Images were collected at 0.5 second intervals.

### (iv) Transmission Electron Microscopy

Cells were grown in rich medium until stationary phase, harvested, fixed with 2.5% glutaraldehyde and postfixed with 2% (w/v) osmium tetroxide. The samples were subsequently dehydrated in a series of graded ethanol and embedded in Spurr's Resin. 80 nm ultrathin sections were stained with uranyl acetate and lead citrate and examined under a JEM-1230 Joel electron microscope.

Total RNA was extracted using the RNeazy Plus kit (QIAGEN). cDNA was generated from total RNA using a SuperScript VILO cDNA Synthesis Kit (Invitrogen). PCR reaction was performed using Rotor-Gene RG-3000A (Qiagen). Threshold cycle value for each gene was acquired at the log phase and gene expression was normalized to reference genes as indicated. For Affymetrix Array processing and analysis, samples were prepared according to the Affymetrix GeneChip® Yeast Genome 2.0 Array protocol. Differences between WT and fld1Δ strains were compared using one-way ANOVA and adjusted for false discovery rate at 0.05 level. Array data is deposited on Gene Expression Omnibus (http://www.ncbi.nlm.nih.gov/geo/).

### (v) Isolation of Microsomes

Microsomes were isolated as described in (see Rieder and Emr S D, (2001) “Isolation of subcellular fractions from the yeast Saccharomyces cerevisiae”, Curr Protoc Cell Biol Chapter 3: Unit 3 8). Briefly, WT and fld1Δ cells were cultured in SC media till log phase (OD600˜1.0) and harvested. Cells as 0.1 g (wet weight)/ml in 0.1 M Tris.SO4 were sequentially incubated in pH9.4/10 mM DTT for 10 min, and in 1.2 M sorbitol/20 mM Tris.Cl, pH7.5/1×SC medium/zymolase 100 T (15 mg/ml) for 30 min. Spheroplasts were lysed in HEPES lysis buffer (10 mM HEPES/KOH, pH6.8/50 mM potassium acetate/100 mM sorbitol/2 mM EDTA) with the aid of a Dounce homogenizer. After removal of cell dedris, lysates were centrifuged at 30 000 g for 10 min at 4° C. P30 000 g membrane pellets were resuspended in HEPES lysis buffer, loaded onto 1.2 M/1.5 M sucrose (prepared in HEPES lysis buffer) gradients, and centrifuged at 100 000 g for 1 h at 4° C. ER membranes were collected at the 1.2 M/1.5 M sucrose interface.

### (vi) Isolation of Lipid Droplets

Lipid droplets were isolated as described in Fei et al. (Fei et al., (2008), “Fld1p, a functional homologue of human seipin, regulates the size of lipid droplets in yeast”, J Cell Biol 180: 473-482).

(vii) Isolation and Analyses of Lipids.

Lipids were extracted from zymolase-digested lyophilized yeast cells, isolated ER microsomes or lipid droplets. Briefly 900 μl ice-clod chlorofom:methanol (1:2) was added to samples. Mixtures were vigorously vortexed for 1 min, and incubated for 2 h in vacuum container with rotary shaking at 4° C. Then 400 μl ice-cold water and 300 μl chloroform were added, vortexed and incubated on ice for 1 min. After centrifugation at 12000 rpm for 3 min at 4° C., the lower organic phase was collected. Subsequently, 50 μl 1 M HCl and 500 μl chloroform were added to the remainder, vortexed, and incubated on ice for 3 min. The lower organic phase was also collected after centrifugation at 12000 rpm for 3 min at 4° C., and combined with the first extract. The extracted lipids were blown dry with nitrogen gas, and resuspended in solvent for mass spectrometry analysis.

Lipidomic analysis, and quantitative measurement of neutral lipids via thin layer chromatography (TLC) were performed as described in Fei et al. (Fei et al., (2008), “Fld1p, a functional homologue of human seipin, regulates the size of lipid droplets in yeast”, J Cell Biol 180: 473-482) and Low et al. (Low et al., (2008), “Caspase-dependent and -independent lipotoxic cell-death pathways in fission yeast”, J Cell Sci 121: 2671-2684). TLC plates were developed in chloroform/methanol/water (65:25:4) to separate phospholipid species.

(viii) In Vitro Fusion Assay of Artificial Lipid Droplets.

To prepare lipid emulsions, lipids were mixed in chloroform/methanol (2:1), dried under a stream of N2, resuspended in buffer (150 mM NaCl, 50 mM Tris/HCl, pH 7.5, 1 mM EDTA,), and sonicated. For emulsions, the molar ratio of TAG to total phospholipids was 2:2.5. Contaminating vesicles were removed, and LDs were concentrated by ultracentrifugation at 100,000 g for 15 min. For light scattering, lipid concentration was 25 mM phospholipids and 20 mM TAG before centrifugation.

### (ix) Statistical Analysis

All data are presented as mean±SD. Statistical comparison between the two groups was performed using Student's t-test. Microarray data were analyzed using one-way ANOVA and adjusted for false discovery rate at 0.05 level.

### Results and Discussion

(i) Identification of Additional Yeast Mutants with “supersized” LDs

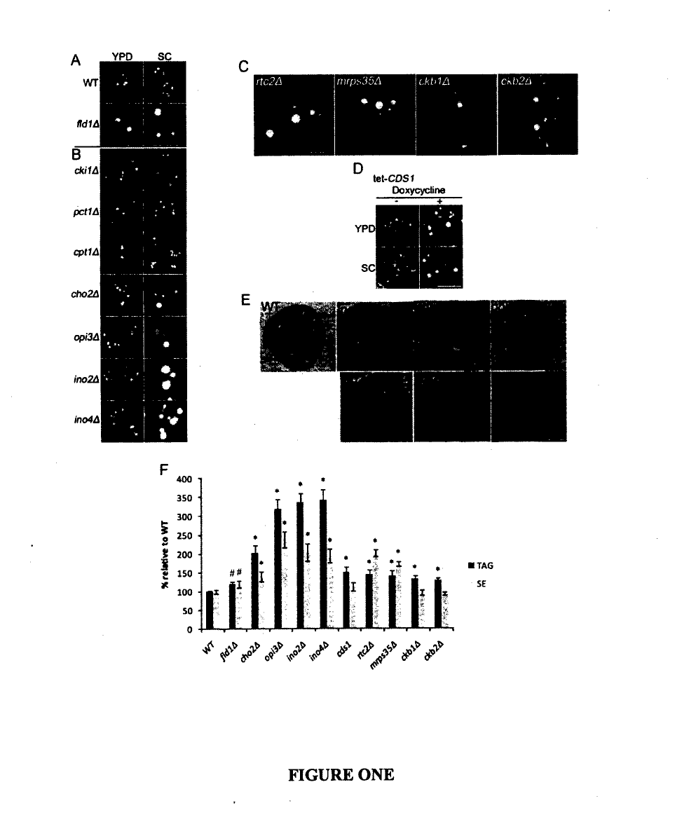

A yeast mutant gene was identified fld1Δ that was observed to developed very large LDs (FIG. 1A). Whereas the diameter of LDs in wild type (WT) cells typically ranges between 0.3 to 0.4 μm, and rarely exceeds 0.5 μm (see Czabany et al. (2008), “Structural and biochemical properties of lipid particles from the yeast Saccharomyces cerevisiae”, J Biol Chem 283: 17065-17074), fld1Δ cells often synthesize LDs with a diameter larger than 1.0 μm. LDs with a diameter greater than 1.0 μm are arbitrarily described as “supersized” LDs (SLDs), whose volume is over 30 times the average of wild type LDs. About 20% of fld1Δ cells cultured in rich (YPD) medium contained SLDs, and the percentage increased to ˜70% when cells were grown in minimal (synthetic complete/SC) medium (FIG. 1A and Table 1).

TABLE 1
Yeast gene deletions that lead to the formation of supersized LDs (SLDs).

| | | | | % of cells with | |
|---|---|---|---|---|---|
| | | | | SLDs* | |
| | | | | SC | YPD |
| SLD | ORF | Gene | Function | media | media |
| 1 | YLR404W | FLD1, | Unknown | 66.1 ± 1.0 | 22.3 ± 3.7 |
| | | SEI1 | | | |
| 2 | YGR157W | CHO2 | PE methyltransferase | 60.9 ± 5.8 | 0.5 ± 0.5 |
| 3 | YJR073C | OPI3 | Phospholipid methyltransferase | 89.3 ± 3.2 | 0.7 ± 0.3 |
| 4 | YDR123C | INO2 | Transcription factor | 97.0 ± 1.0 | 0.3 ± 0.3 |
| 5 | YOL108C | INO4 | Transcription factor | 96.3 ± 1.6 | 0.5 ± 0.0 |
| 6 | YBR147W | RTC2 | Unknown | 71.0 ± 2.7 | 0.7 ± 0.3 |
| 7 | YGR165W | MRPS35 | Unknown | 69.3 ± 5.1 | 0.3 ± 0.3 |
| 8 | YGL019W | CKB1 | Beta regulatory subunit of casein | 32.8 ± 3.4 | 0.5 ± 0.5 |
| | | | kinase 2 | | |
| 9 | YOR039W | CKB2 | Beta′ regulatory subunit of casein | 20.0 ± 3.2 | 0.7 ± 0.3 |
| | | | kinase 2 | | |
| 10 | YBR029C | CDS1 | CDP-DAG synthase | 46.6 ± 3.7 | 29.1 ± 5.6 |
| WT | | | | 0.7 ± 0.3 | 0.3 ± 0.3 |

Cells were grown in synthetic complete (SC) media or rich YPD media to stationary phase, stained with Nile red, and observed under a fluorescence microscope for LDs. Experiments were done in triplicates and ~200 cells were counted for all strains each time. The percentages of cells that displayed SLDs were represented as mean ± SD. SC has 11 μM inositol because it contains yeast nitrogen base.

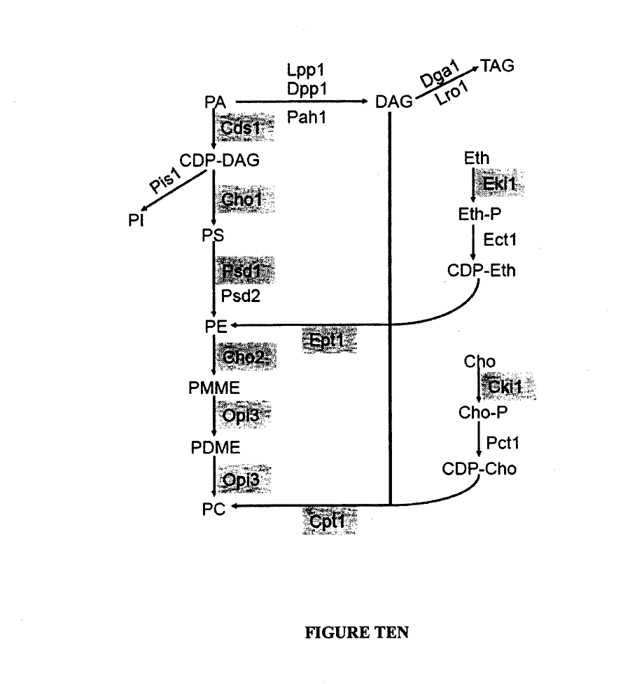

Given the effects various nutrients may have on the dynamics of LDs, it was hypothesised that growing cells on defined, minimal media (SC) would reduce the impact of nutrients and uncover additional genes. The entire collection of viable yeast deletion mutants (˜4800) grown on minimal (SC) media for SLDs was screened. In order to identify essential genes impacting LD size, the collection of mutants where all essential genes are controlled by the TetO₇-promoter, which can be switched off efficiently, were included (see Mnaimneh et al. (2004) “Exploration of essential gene functions via titratable promoter alleles”, Cell 118: 31-44). Besides fld1Δ, nine additional mutants (sld2-10) were identified that produced supersized LDs (SLDs) (FIG. 1 and Table 1). Except for two previously uncharacterized genes (RTC2/SLD6&MRPS35/SLD7), the majority of the SLD genes appear to function directly or indirectly in the metabolism of phospholipids, especially phosphatidylcholine (PC). PC is synthesized in yeast via two pathways: the Kennedy pathway and the phosphatidylethanolamine N-methyltransferase (PEMT) pathway (see FIG. 10). In the PEMT pathway, phosphatidylethanolamine (PE) is methylated to PC in three steps by two methyltransferases, Cho2p and Opi3p. Besides cho2Δ and opi3Δ mutants, ino2Δ and ino4Δ mutants are also defective in PC synthesis via PE methylation since Ino2p and Ino4p are transcription factors that positively regulate the PEMT pathway. When cki1Δ, pct1Δ, cpt1Δ, cho2Δ, opi3Δ, ino2Δ, and ino4Δ cells were cultured in rich (YPD) medium, none of these mutants accumulated SLDs. In contrast, when grown in SC medium (no choline and hence little Kennedy pathway activity), approximately 60% of cho2Δ cells, 90% of opi3Δ cells, 97% of ino2Δ and ino4Δ cells produced SLDs, whereas cki1Δ, pct1Δ, and cpt1Δ cells did not (FIGS. 1B&E). From these results, PC synthesis does appear to be critical in regulating the size of LDs. Interestingly, of the 825 essential genes examined, SLDs were observed only upon knocking-down CDS1 (encoding CDP-diacylglycerol synthase) (FIGS. 1D&E). Therefore, the synthesis of not only PC, but also other phospholipids could be important for LD growth. The SLDs observed in fld1Δ, cho2Δ, opi3Δ, ino2Δ, ino4Δ, and TetO₇-CDS1 (thereafter referred to as cds1 when repressed by doxycycline) strains were further confirmed by electron microscopy (FIG. 1E). The levels of TAG and SE of all mutants were also examined, and the level of TAG was significantly increased in all mutants (FIG. 1F).

### (ii) Defective TAG Mobilization of “Supersized” LDs

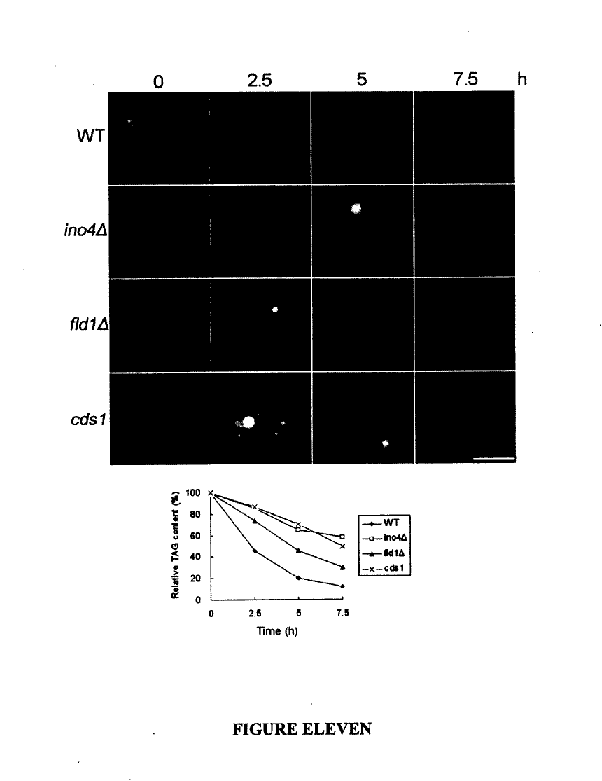

As compared to many small LDs in WT cells, the formation of SLDs limits the surface area that is accessible to lipases. Therefore, the mobilization of TAG may be impaired in sld mutants. TAG breakdown in fld1Δ, ino4Δ, and cds1 strains was monitored in the presence of 10 mg/L cerulenin that prevents their de novo synthesis (see Kurat et al., (2009), “Cdk1/Cdc28-dependent activation of the major triacylglycerol lipase Tgl4 in yeast links lipolysis to cell-cycle progression”, Mol Cell 33: 53-63. These results show that TAG mobilization in the mutants is significantly slower than that of WT (FIG. 11).

(iii) Treatment of Different Phospholipid Precursors Exerted Distinct Effects on the Formation of SLDs

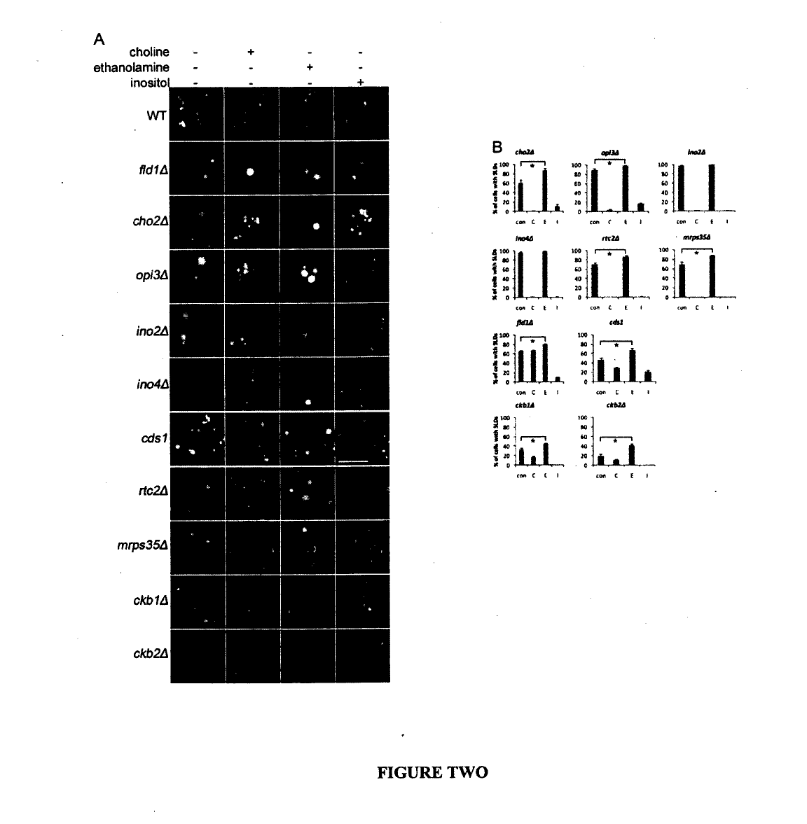

The finding that YPD media invariably decreased the percentage of cells displaying SLDs in all sld mutants suggested that certain components present in rich YPD media, but absent or low in SC media, suppressed SLD formation. Considering that Cds1p, Cho2p, Opi3p, Ino2p, and Ino4p are either enzymes or transcription factors involved in phospholipid biosynthesis, it was speculated that these components might be precursors of phospholipids. To examine this possibility, WT and mutant strains were cultured in SC media supplemented with 1 mM choline, 1 mM ethanolamine, or 75 μM inositol. Interestingly, inositol treatment reduced the SLD formation in all mutants (FIG. 2). In contrast, ethanolamine addition had an opposite effect; it enhanced SLD formation in most of the mutants. As expected, choline addition completely blocked the formation of supersized LDs in cho2Δ, opi3Δ, ino2Δ and ino4Δ strains (FIGS. 2A&B), since exogenously added choline restored PC synthesis in these mutants through the Kennedy pathway. Surprisingly it also had similar effect in rtc2Δ and mrps35Δ strains, suggesting that these two genes may also function in PC metabolism. Choline addition also partially inhibited SLD formation in cds1, ckb1Δ, and ckb2Δ cells, but had little effect in fld1Δ cells.

### (iv) a Link Between the Generation of “Supersized” LDs and an Elevated Level of Intracellular Phosphatidic Acid (PA)

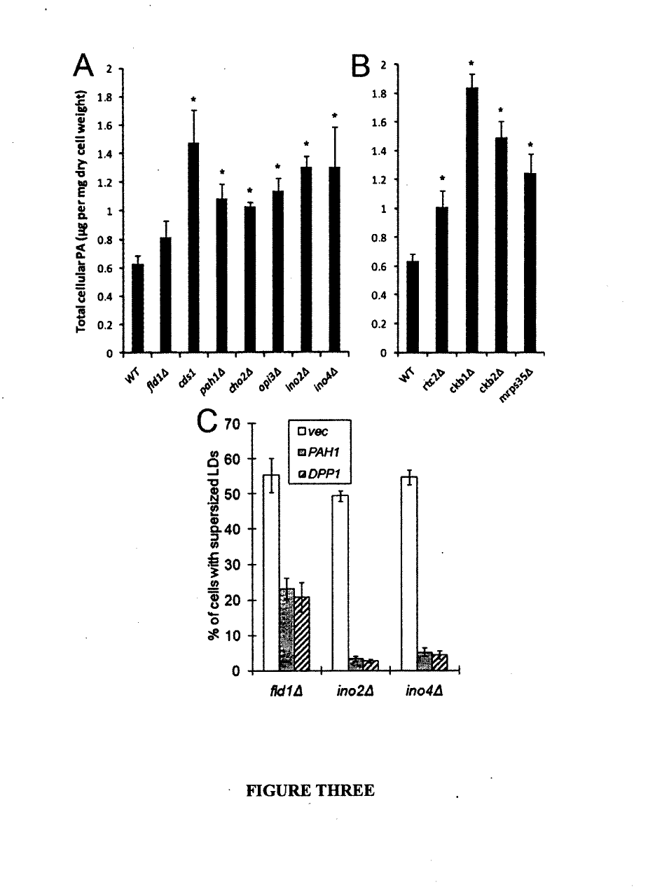

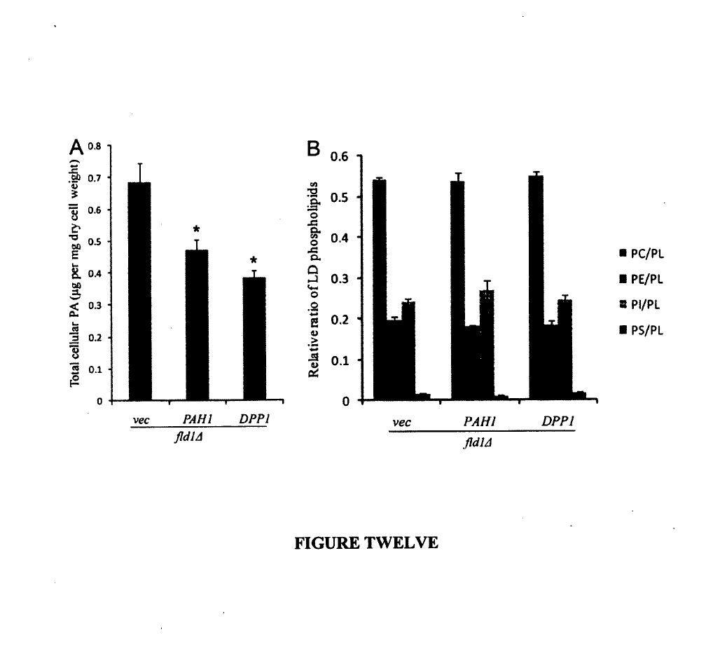

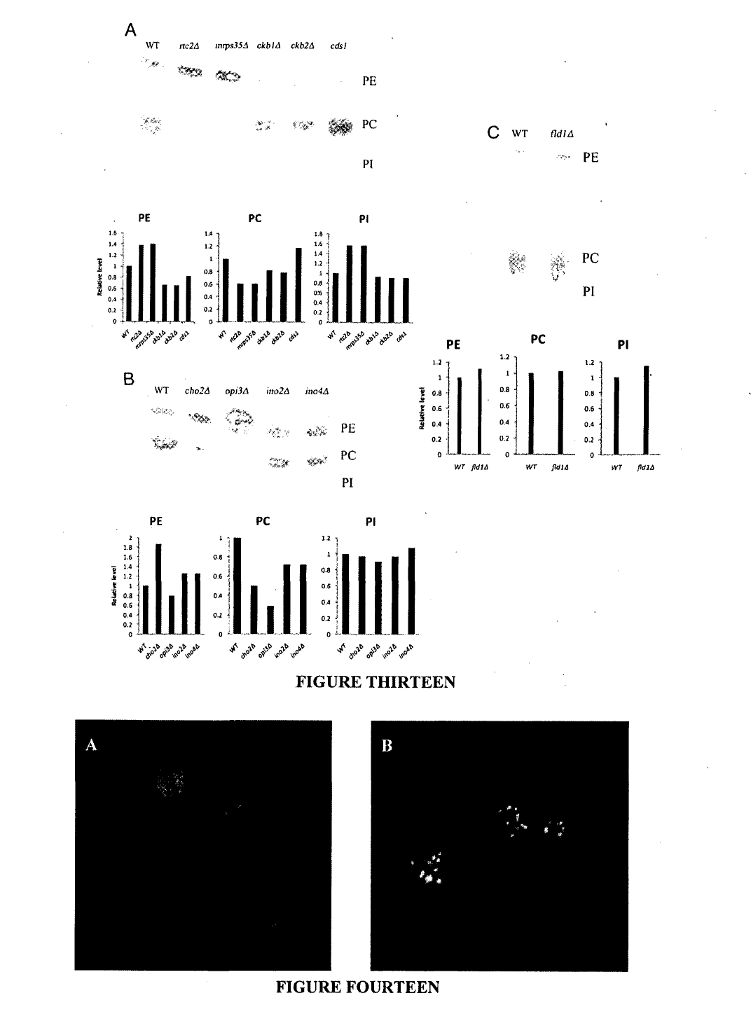

One notable common feature among cho2Δ, opi3Δ, ino2Δ, ino4Δ and cds1 mutants is the accumulation of PA (FIG. 3A). PA is a cone-shaped lipid that alters the curvature of the membranes, promotes both SNARE-dependent and -independent membrane fusion events, and is implicated in the assembly of lipid droplets from newly synthesized TAGs. To examine whether PA is a key player in the formation of SLDs, we first analyzed the cellular level of PA in the SLD mutants by LC-MS. Indeed a significant elevation of PA was seen in all mutants except fld1Δ cells, where the level of PA is only moderately elevated (FIG. 3A&B). Inositol treatment reduces the cellular PA pool through increased synthesis of phosphatidylinositol (PI) and also through the activation of a Mg²⁺-dependent PA phosphatase. Consistent with the implication of PA in SLD formation, inositol treatment resulted in a significant reduction of SLD formation in all mutants including fld1Δ (FIG. 2). In addition, when two PA phosphatases (PAH1 and DPP1) were overexpressed under a GAL1 promoter, both greatly reduced SLD formation in ino2Δ and ino4Δ cells, and also in fld1Δ cells (FIG. 3C). Overexpression of PAH1 and DPP1 did not change the level of PC, PE, PS and PI, but significantly reduced the cellular level of PA in fld1Δ cells (FIG. 12). These results imply that the increased amount of PA may account for the formation of SLDs, and that the level of PA in subcellular organelles such as the endoplasmic reticulum where LDs originate may have changed in fld1Δ cells, despite an insignificant increase in overall PA in this mutant (see below).

(v) Fld1p could Regulate the Metabolism of Phospholipids

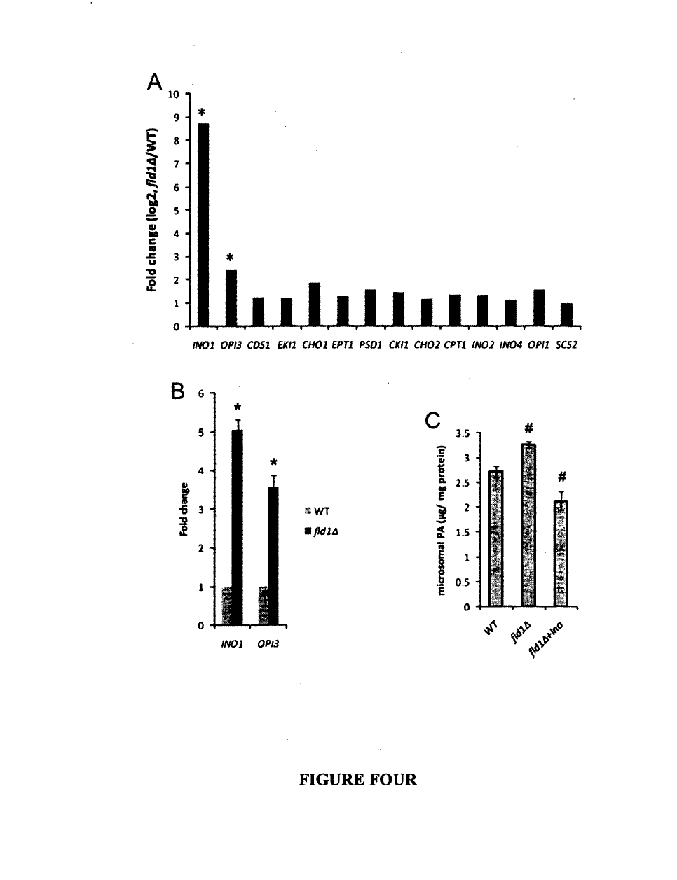

SLDs in yeast were originally identified in fld1Δ cells; but the molecular function of Fld1p/seipin remains elusive (Fei et al., (2008), “Fld1p, a functional homologue of human seipin, regulates the size of lipid droplets in yeast”, J Cell Biol 180: 473-482). To gain more insights into the function of Fld1p, mRNA microarray analysis was performed in WT and fld1Δ cells. Of ˜5800 transcripts examined, INO1 and OPI3 were the only transcripts whose levels were significantly upregulated in fld1Δ cells (FIG. 4A). Quantitative real-time PCR confirmed a ˜5-fold increase in the INO1 mRNA level in fld1Δ cells (FIG. 4B). INO1 gene expression is derepressed when intracellular PA concentration rises. It was therefore examined the level of PA on the ER where the Opi1p-Scs2p regulatory complex of INO1 expression exists. Indeed, a significant increase of PA was observed in microsomes isolated from fld1Δ cells (FIG. 4C). These results show that Fld1p, as other SLD mutants, can regulate PA metabolism, and also suggest that both the level and location of PA are relevant to droplet formation.

### (vi) Elevated PA Enhances SLD Formation in Yeast

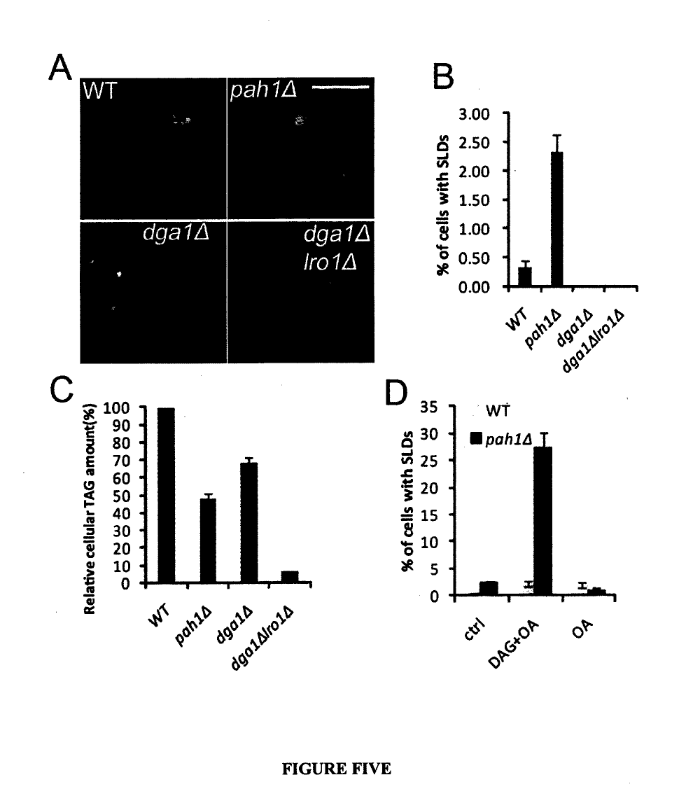

Another strategy to increase PA is through inactivation of Pah1p, the PA phosphatase and ortholog of mammalian lipin proteins. Deletion of PAH1 leads to a dramatic increase in the level of its substrate, PA, but causes a dramatic reduction in the amount of TAG. Although the number of LDs was significantly reduced in pah1Δ cells, LD size was comparable to that in WT cells. Remarkably, SLDs were detected consistently in ˜3% of pah1Δ cells, though its TAG synthesis was decreased by over 50% (FIG. 5A-C). In contrast, no SLDs were ever observed in dga1Δ lro1Δ cells, which have little diacylglycerol (DAG) acyltransferase activity. Interestingly, when pah1Δ cells were supplemented with oleate and DAG which bypasses the lack of PA phosphatase activity, the number of cells producing SLD increased to ˜30%, whereas oleate alone had no effect (FIG. 5D). These results further indicate that PA plays an important role in SLD formation, and this role is more pronounced when the biosynthesis of TAG is not severely compromised.

(vii) Elevated Phosphatidylethanolamine Concentration is a Factor in the Biogenesis of SLDs

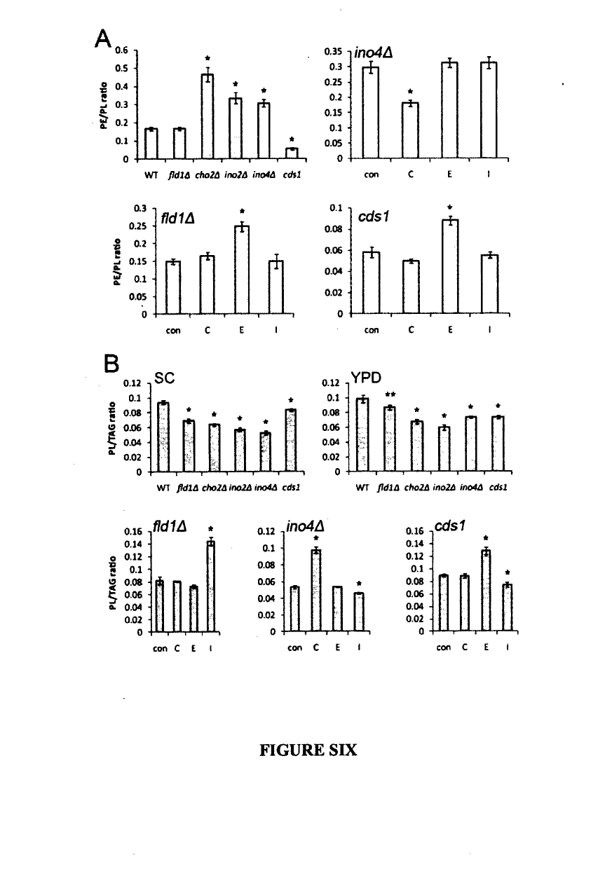

The result that ethanolamine addition enhanced SLD formation in nearly all mutants (FIG. 2) suggested that an elevated phosphatidylethanolamine (PE) concentration could have a role in SLD formation, given that PE is also a cone-shaped phospholipid that can increase membrane curvature, thereby promoting LD monolayer coalescence. Consistent with this notion, mutants that accumulate PE also displayed a higher percentage of cells forming SLDs, particularly opi3Δ, ino2Δ, and ino4Δ (Table 1). As shown in FIG. 6A, the lipidomic analysis further revealed that lipid droplets isolated from cho2Δ, ino2Δ, and ino4Δ also had a higher PE to PL (total membrane phospholipids) ratio than those of WT cells. In addition, ethanolamine treatment significantly increased the proportion of PE on lipid droplets of fld1Δ and cds1 cells. Even in LDs of ino4Δ cells, ethanolamine addition still moderately increased the PE to PL ratio, though its PE level was already much higher than WT. However, elevated PE alone was not able to induce SLD formation since inositol addition completely abolished the biogenesis of supersized LDs despite that a higher PE to PL ratio persisted in ino4Δ cells (FIG. 6A). Moreover, SLDs were abundant in cds1 where the PE/PL ratio was lower than that of the WT (FIGS. 6A &13).

(viii) Reduced PL to TAG Ratio does not Completely Correlate with the Formation of SLDs

A decreased phospholipid (PL) to TAG ratio could also induce SLD formation, since coalescence may be induced to decrease the surface-to-volume ratio of droplets when phospholipids are limiting. This model appears to be true for the mutants grown in SC media. However, when grown in YPD media, SLDs disappeared in cho2Δ, ino2Δ and ino4Δ strains but the decreased PL to TAG ratio persisted (FIG. 6B). In addition, inositol supplementation did not increase the phospholipid to TAG ratio in cds1 or ino4Δ mutant (FIG. 6B).

### (ix) Rtc2p and Mrps35p, New Players in Phospholipid Metabolism

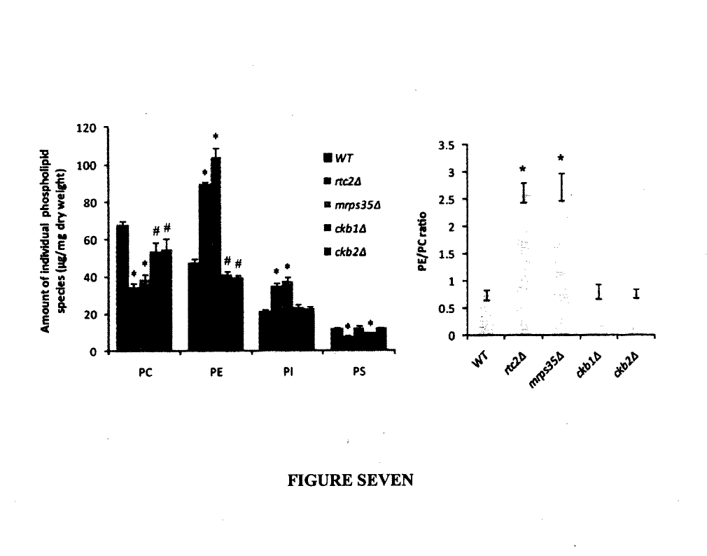

Choline addition completely inhibited SLD formation in rtc2Δ and mrps35Δ, in a manner similar to cho2Δ, opi3Δ, ino2Δ, and ino4Δ, strains known to be defective in the methylation of PE into PC (FIG. 2). This phenotype suggests that deletion of RTC2 or MRPS35 might affect PC synthesis through the PEMT pathway. As expected, lipidomic analysis revealed that rtc2Δ and mrps35Δ strains displayed a 2.5-fold increase of PE to PC ratio, indicating these two gene products are involved in PC synthesis through the PEMT pathway (FIG. 7). rtc2Δ and mrps35Δ cells also synthesized ˜60% more phosphatidylinositol (PI) than WT, possibly resulting from the accumulation of CDP-DAG due to a blocked PEMT pathway (FIGS. 7&13A). The phospholipid profiles of ckb1Δ and ckb2Δ strains were also examined and found that both synthesized less PC and PE than WT without causing significant changes in the PE to PC ratio (FIGS. 7&13A).

### (x) LDs of Sld Mutants Demonstrated Enhanced Fusion Activities Both In Vivo and In Vitro

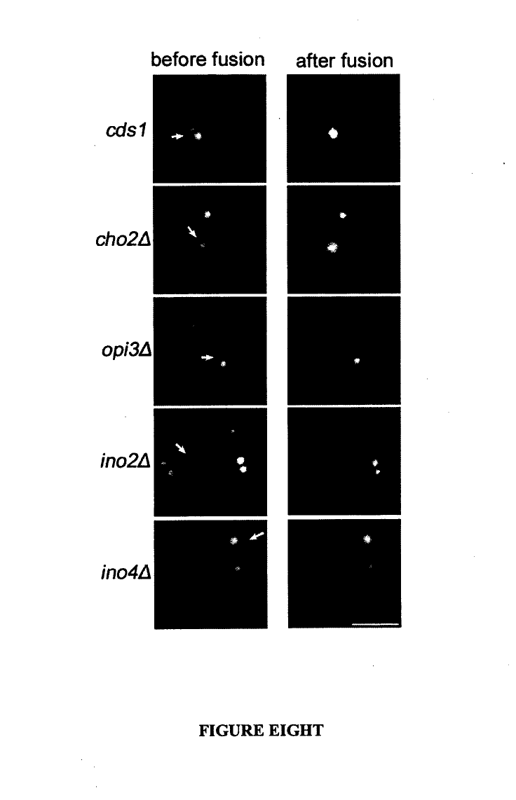

It was next investigated how changes in phospholipids in the sld mutants may lead to the formation of SLDs. One possibility could be enhanced fusion activities. Indeed, fusion of Nile red-stained LDs could be observed in cho2Δ, opi3Δ, ino2Δ, ino4Δ, and cds1 strains (FIG. 8), and also in rtc2Δ, mrps35Δ, ckb1Δ and ckb2Δ strains (data not shown). The fusion frequency of LDs in each mutant was similar (˜10 out of 200 adjacent pairs of LDs) to that of fld1Δ. Furthermore, LDs isolated from representative strains demonstrated fusion activities in vitro (videos S3 and S4). It should be noted that no fusion events in wild type yeast cells were ever observed with the methods employed.

### (xi) Phosphatidic Acid Induces Fusion of Artificial LDs In Vitro

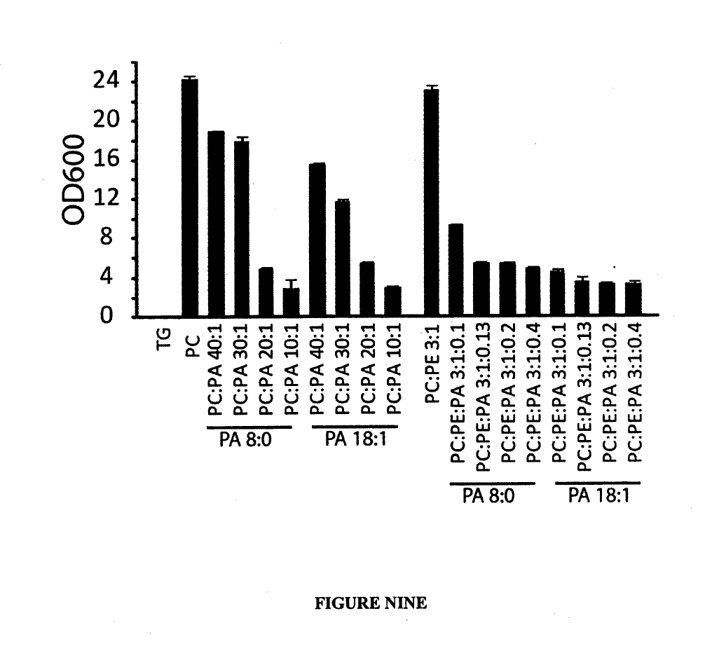

To obtain direct evidence that PA induces coalescence of small LDs to form supersized droplets, artificial LDs were made and their stability tested. After generating the artificial droplets by sonication, we removed liposomes formed at the same time by density gradient centrifugation. This fractionation also concentrated the artificial droplets. From this starting point, we followed the stability of LDs by light scattering, which directly measures the number of LDs. When the concentration of PA in artificial LDs was increased, their number decreased significantly during incubation (FIG. 9). In the presence of PE, a smaller fraction of PA (˜3% molar ratio, PC:PE:PA 3:1:0.13) achieved a similar effect compared to the coalescence observed in the presence of PC covered LDs (˜5% PA (PC:PA 20:1). These results show that PA reduces the stability of LDs and mediates coalescence of LDs, and that this property of PA may be modulated by the phospholipid composition of LDs.

(xii) Discussion

Lipid droplets are dynamic organelles whose number and size undergo constant changes in response to internal and external cues. The physiological relevance of the size of the LDs is not well understood, and far less is known about how the size of LDs is determined at the molecular level. In this study, key proteins were identified that govern the size of LDs in yeast by modulating phospholipid metabolism. Also identified were proteins previously unknown to regulate phospholipid metabolism. Most importantly, in vivo and in vitro evidence is provided that phosphatidic acid can influence the size of the LDs.

SLDs provide an efficient form of fat storage in terms of surface to volume ratio. An interesting question is whether cells automatically generate SLDs upon lipid loading to economize on the synthesis of phospholipids that form the surface of lipid droplets. While large lipid droplets are typical in white adipocytes, most other cell types (brown adipocytes, hepatocytes, myocytes) store lipids in numerous small LDs. In WT yeast cells, a dramatic increase in the number but not size of LDs is often observed in growth conditions favoring neutral lipid synthesis and storage, such as starvation. Maintaining small LDs may be physiologically important: upon starvation, yeast cells convert phospholipid intermediates and sterols to neutral lipids which in turn can be hydrolyzed to release fatty acids and sterols for immediate membrane synthesis and cell growth when glucose becomes available. Lipolysis may be important for efficient cell-cycle progression in yeast and lipolysis occurs more efficiently for small LDs (FIG. 11). Therefore, it appears that SLDs are only formed in highly specialized, non-dividing cells (e.g. fully differentiated white adipocytes) or under pathological conditions such as severe hepatic steatosis.

Genetic factors that regulate the size of LDs were identified in a screen of yeast. Decreased PC synthesis, and consequently an increased PE to PL ratio (or a decrease in PC/TAG), has been associated with SLD formation (FIG. 1). LDs are phase-separated organelles in the cytoplasm. Thus, unlike for other organdies, the steady state and lowest energy state is probably to have only one droplet (this would minimize interfacial surface energy). Some phospholipids (specifically PC) shield droplets from coalescence. Fusogenic lipids such as PA and PE could overcome this effect. In agreement with this notion, it was identified that PE and PA both have an effect on SLD formation. First, treatment of ethanolamine further increased PE to PL ratio and enhanced SLD formation in most mutants (FIGS. 2&6); in addition, strains with increased levels of both PE and PA also had a higher percentage of cells producing supersized LDs (Table 1). Decreased levels of PLs also could lead to SLD formation, since phospholipids levels may not be sufficient under these conditions to prevent the hydrophobic TAG phases to fuse. If the amount of PLs on LDs is not sufficient, fusion would occur until the surface-to-volume ratio of LDs is reflecting the ratio of phospholipids to TAG. At this point the monolayer would shield the LD from any further fusion.

Identification of the cds1 mutant through the screening of the knock-down collection of essential yeast genes brought attention to PA, whose critical role in SLD formation was confirmed by the strong “size-reduction” effect of inositol supplementation, an efficient and reliable way to reduce PA in yeast. The essential role of PA in SLD formation was further confirmed when all mutants that develop SLDs were found to accumulate PA (FIG. 3), including the seipin-deficient (fld1Δ) (FIG. 4) and lipin-deficient (pah1Δ) (FIGS. 5A&B) cells. PA is a central intermediate in the synthesis of major glycerolphospholipids and TAG, as well as an important signaling lipid. Different pools of PA and distinct PA subclasses may account for the diversity of PA function. For instance, the yeast Opi1p (ER localized transcription repressor) senses only a PA pool on the ER but not the plasma membrane PA pool regulated by the yeast phospholipase D Spo14p. Deleting or overexpressing SPO14 did not have any impact on the formation of SLDs (data not shown), suggesting that an intracellular (ER/LD) PA pool is responsible for SLD formation. This appears to be also the case for the fld1Δ mutant, in which the level of microsomal PA, but not overall PA, is significantly increased. The level of PA on the ER could very well reflect the amount of PA on the LDs, given that LDs are believed to originate from the ER. In summary, it was found that increased PA levels may overcome the effect of phospholipid shielding, and that the location of PA also matters.

Besides establishing a strong link between PA and the size of LDs, these results provided herin also reveal that Rtc2p and Mrps35P can regulate the PEMT pathway of PC synthesis. Both Rtc2p and Mrps35p associate with mitochondria. Exactly how ckb1Δ and ckb2Δ mutants cause a significant increase in cellular PA and thereby the formation of SLDs is not clear, although the key transcription factor that regulates phospholipid synthesis in the yeast, Opi1p, can be phosphorylated and regulated by casein kinase 2.

Finally, the mechanistic link between changes in the level of PA/PE and the formation of SLDs was investigated. LDs are covered by a monolayer of phospholipids, whose composition may have a profound effect on the dynamics of the LDs. Both PA and PE are cone-shaped, fusogenic lipids that can alter the curvature of the membranes. PA, in particular, promotes both SNARE dependent and independent membrane fusion events. It is possible that a higher level of PA on the monolayer of the LDs would promote spontaneous fusion of contacting LDs. Indeed, in vivo microscopic observation found increased incidents of LD fusion in mutants with increased level of PA (FIG. 8). Remarkably, when artificial LDs are made with different ratios of PA, PE and PC, it is clear that even a small amount of PA could significantly increase the size of the LDs (FIG. 9).

In summary, studies described herein identify novel protein and lipid regulators of the size of the LDs, an important lipid-storage organelle. Knowing how LD size is determined may provide invaluable insights into how human cells/tissues handle abnormal influx of lipids in today's obesogenic environment.

### Example 2

### Knockdown of Gene Expression of CDS1p Homologue in the Green Alga Chlamydomonas Increases Lipid Droplet Accumulation

### Materials and Methods

### (i) Algal Strains

Chlamydomonas reinhardtii wild type strain 21 gr.

### (ii) RNAi Construct and Transformation

A hairpin RNAi construct was made by using 1^{st}-3^{rd} exon of the Chlamydomonas homologue of CDS1p driven by LC8 promoter. The gene name of the homologue is C_{—}30067. The LC8 promoter is from Chlamydomonas.

The sequence of the first three exons of the gene is the following:

(SEQ ID NO: 41)

ATGCGACCAAAACAACAAGCTCAGGTCACCGCCGCGGAGGACACCGCGTC
GGAGCCGGAGCCGGAGCCCAAGCCCGTGGATCGAAAGTCGAGGCTCAGGT
CCTTCCGCGTACGCACCATCTCCTCGGTCTTGCTCATCGGCGGGTTCATC
GGCATCATTTGGGCGGGTCATGTGCCGCTTATGTTCTTCATCCTGCTACT
CCAGTTCCTGGTGGCCCGGGAGCTCTTCCGCATCGCCTACATAGCGGAGA
AGTCCAAACGCAGCC

This construct was transformed into Chlamydomonas using a glass-bead method. The selection marker for transformation was APHVIII, whose expression conferred resistant to paromomysin.

(iii) RNAi Strain Selection

The survival strains on agar plates were screened by using Nile Red staining. 1 ug/ml final concentration of Nile red was used.

### (iv) Microscopic Observation

A Zeiss fluorescence microscope (Observer.Z1) with GFP filter was used for screening putative RNAi strains.

### Results

It was observed that Chlamydomonas reinhardtii transformed with the CDS1 gene RNAi accumulated lipid droplet drastically. FIG. 14 shows typical fluorescent images of wild type (A—left image) and the RNAi strain (B—right image).

### Summary

CDS1p is a key enzyme in lipid metabolism. To understand whether it plays a role in lipid droplet accumulation in algae, expression of the CDS1p homologue in Chlamydomonas was knocked down and droplet accumulation analyzed. It was observed that lipid droplet increases drastically in the RNAi strains.

### Example 3

### Identification of Genes in Brassica sp. (Mustard), Ricinus sp. (Castor Bean), and Glycine sp. (Soybean) Homologous to S. cerevisiae Genes

1. Brassica sp. (mustard) Vs S. cerevisiae Alignments:

| * Cds1 | | | |
|---|---|---|---|
| SEQ ID NO: 1 Brassica sp. XA_0051 (Cds1 homologue) | | | |
| DKNKYRSMWIRTCSSLWMLGGVFFIIYMGHLYIWAMVVGIQIFMAKELFFLLRRAHEERRLPGFRLLNWHFFFTA | | | |
| MLFVYGRILQQQLVNTVSSDRFIYKLVSGLIKYQMVICYFLYIAGFMWFILTLKKKMYKYQFGQYAWTHMILIVV | | | |
| FTQSSFTVANIFEGIFWFLLPAALIAMNDVAAYFFGFYFGKTPLIKLSPKKTWEGFIGA | | | |
| >XA_0051 Length = 1633677 | | | |
| E-value = 1.37e−34, Score = 367, Bitscore = 145.976, | | | |
| Identities = 77/209 (36%), Positives = 122/209 (58%), Gaps = | | | |
| 5/209 (2%) | | | |
| XA_0051: 350453-350661 = SEQ ID NO: 1 | | | |
| Cds1 | 56 | ESRKY-NFFIRTVWTFVMISGFFITLASGHAWCIVLILGCQIATFKECIAVTSASGREKN | 114 |
| | | + KY + +IRT + M+ G F + GH + +++G QI KE + + E+ | |
| XA 0051 | 350661 | DKNKYRSMWIRTCSSLWMLGGVFFIIYMGHLYIWAMVVGIQIFMAKELFFLLRRAHEERR | 350602 |
| Cds1 | 115 | LPLTKTLNWYLLFTTIYYLDGKSLFKFFQATF----YEYPVLNFIVTNHKFICYCLYLMG | 170 |
| | | LP + LNW+ FT + ++ G+ L + T + Y +++ ++ ICY LY+ G | |
| XA 0051 | 350601 | LPGFALLNWHFFFTAMLFVYGRILQQQLVNTVSSDRFIYKLVSGLIKYQMVICYFLYIAG | 350542 |
| Cds1 | 171 | FVLFVCSLRKGFLKFQFGSLCVTHMVLLLVVFQAHLIIKNVLNGLFWFLLPCGLVIVNDI | 230 |
| | | F+ F+ +L+K K+QFG THM+L++V Q+ + N+ G+FWFLLP L+ +ND+ | |
| XA 0051 | 350541 | FMWFILTLKKKMYKYQFGQYAWTHMILIVVFTQSSFTVANIFEGIFWFLLPAALIAMNDV | 350482 |
| Cds1 | 231 | FAYLCGITFGKTKLIEISPKKTLEGFLGA | 259 |
| | | AY G FGKT LI++SPKKT EGF+GA | |
| XA 0051 | 350481 | AAYFFGFYFGKTPLIKLSPKKTWEGFIGA | 350453 |
| * Ckb1 | | | |
| A. SEQ ID NO: 2 Brassica sp. XA_0048r (Ckb1 homologue) | | | |
| SEVSGSDEEDLAWTTWFCKLPGNEFLCEVDDCFILDNFNLCGLRHQVPFYDNALDLILDDDSSS | | | |
| >XA_0048r Length = 1689678 | | | |
| E-value = 1.15e−34, Score = 127, Bitscore = 53.5286, | | | |
| Identities = 29/68 (42%), Positives = 38/68 (55%), Gaps = 4/68 | | | |
| (5%) | | | |
| XA_0048r: 346457-346520 = SEQ ID NO: 2 | | | |
| Ckb1 | 10 | SRTGSSDDEDSGAYDEWIPSFCSRFGHEYFCQVPTEFIEDDFNMTSLSQEVPHYRKALDL | 69 |
| | | S SD+ED W FC G+E+ C+V FI D+FN+ L +VP Y ALDL | |
| XA 0048r | 346520 | SEVSGSDEEDLA----WTTWFCKLPGNEFLCEVDDCFILDNFNLCGLRHQVPFYDNALDL | 346465 |
| Ckb1 | 70 | ILDLEAMS | 77 |
| | | ILD ++ S | |
| XA 0048r | 346464 | ILDDDSSS | 346457 |
| B. SEQ ID NO: 3 Brassica sp. XA_0048r (Ckb1 homologue) | | | |
| LIESAAEMLYGLIHARYILTDKGFLSMLNKYNKSEFGRCPRVYCSGQSCLPIGLSDVPGASTVKIYCPKCEDIYH | | | |
| QPSKYQGSSSILLLVRIRYFSHLCVLVFFIHIKTDIDGSYFGTAFPHLFLMYYPSRRPKKVSSQSYVPRVFGFNLH | | | |
| >XA_0048r: Length = 1689678 | | | |
| E-value = 1.15e−34, Score = 281, Bitscore = 112.849, | | | |
| Identities = 61/154 (39%), Positives = 88/154 (57%), Gaps = | | | |
| 31/154 (20%) | | | |
| XA_0048r: 346273-346423 = SEQ ID NO: 3 | | | |
| Ckb1 | 118 | IIEHAAEQLYGLIHARFILTKPGLQAMAEKFDHKEFGTCPRYYCNGMQLLPCGLSDTVGK | 177 |
| | | +IE AAE LYGLIHAR+ILT G +M K++ EFG CPR YC+G LP GLSD G | |
| XA 0048r | 346423 | LIESAAEMLYGLIHARYILTDKGFLSMLNKYNKSEFGRCPRVYCSGQSCLPIGLSDVPGA | 346364 |
| Ckb1 | 178 | HTVRLYCPSCQDLYLPQSSRF-----------------LC-----------LEGAFWGTS | 209 |
| | | TV++YCP C+D+Y Q S++ LC ++G+++GT+ | |
| XA 0048r | 346363 | STVKIYCPKCEDIY-HQPSKYQGSSSILLLVRIRYFSHLCVLVFFIHIKTDIDGSYFGTA | 346305 |
| Ckb1 | 210 | FPGVFLKHFKELEEYVERKSKESYELKVFGFRIN | 243 |
| | | FP +FL ++ ++ S +SY +VFGF ++ | |
| XA 0048r | 346304 | FPHLFLMYYPSRRP--KKVSSQSYVPRVFGFNLH | 346273 |
| * Rtc2 | | | |
| SEQ ID NO: 4 Brassica sp. XA_0011r (Rtc2 homologue) | | | |
| GIVSLICWGVAEIPQIITNFRTKSSHGVSLSFLLAWVAGSVHSII | | | |
| >XA_0011r Length = 4370687 | | | |
| E-value = 2.04e−04, Score = 105, Bitscore = 45.0542, | | | |
| Identities = 18/45 (40%), Positives = 31/45 (68%), Gaps = 0/45 | | | |
| (0%) | | | |
| XA_0011r: 312819-312775 = SEQ ID NO: 4 | | | |
| Rtc2 | 18 | GSISICCWIVVFVPQIYENFRRQSAEGLSLLFIVLWLLGDIFNVM | 62 |
| | | G +S+ CW V +PQI NFR +S+ G+SL F++ W+ G + +++ | |
| XA 0011r | 312775 | GIVSLICWGVAEIPQIITNFRTKSSHGVSLSFLLAWVAGSVHSII | 312819 |
| * Scs2 | | | |
| SEQ ID NO: 5 Brassica sp. XA_0001r (Scs2 homologue) | | | |
| KVELKKQSSCSLQISNKTSTQVVAFKVKTTNPRKYCVRPNTGVVLPGDSCNVTGDQLNCVFLFLFKNIYYTDQLN | | | |
| YVCLFLTLVTMQAQKEAPLDMQCKDKFLV | | | |
| >XA_0001r Length = 10813983 | | | |
| E-value = 2.04e−08, Score = 138, Bitscore = 57.7658, | | | |
| Identities = 39/105 (37%), Positives = 50/105 (47%), Gaps = | | | |
| 31/105 (29%) | | | |
| XA_0001r: 1784455-1784559 = SEQ ID NO: 5 | | | |
| Scs2 | 14 | KSPLTEQSTEYASISN-NSDQTIAFKVKTTAPKFYCVRPNAAVVAPGETI-----QVQVI | 67 |
| | | K L +QS+ ISN S Q +AFKVKTT P+ YCVRPN VV PG++ Q+ + | |
| XA 0001r | 1784559 | KVELKKQSSCSLQISNKTSTQVVAFKVKTTNPRKYCVRPNTGVVLPGDSCNVTGDQLNCV | 1784500 |
| Scs2 | 68 | FLGL-------------------------TEEPAADFKCRDKFLV | 87 |
| | | FL L +E D +C+DKFLV | |
| XA 0001r | 1784499 | FLFLF*KNIYYTDQLNYVCLFLTLVTMQAQKEAPLDMQCKDKFLV | 1784455 |

2. Ricinus sp./Castor Bean (“Sbjct”) Vs S. cerevisiae Matches (“Query”) Alignments:

| * Cds1 | | | |
|---|---|---|---|
| SEQ ID NO: 6 Ricinus sp. 29842.m003508 (Cds1 homologue) | | | |
| HRKRSNEVVPEAAKENGGHLLVDDQNKYKSMWIRTCSTVWMIGSFALIVYMGHLYITAMVVVIQIYMAKELFNLL | | | |
| RKAHEDRHLPGFRLLNWHFFFTAMLFVYGRILSQRLVNTVTSDKFLFQLVNSLIKYHMAMCYFLYIAGFMWFILT | | | |
| LKKKMYKYQFGQYAWTHMILIVVFTQSSFTVANIFEGIFWFLLPASLIVINDIFAYICGFFFGRTPLIKLSPKKT | | | |
| WEGFIGASVTTMISAFVLANMMGRFQWLTCPRKDLSSGWLQCDPGPLFKPEYFILPEWVPQWFPWKEISILPVQW | | | |
| HALWLGLFASIIAPFGGFFASGFKRAFKIKDFGDSIPGHGGITDRMDCQMVMAVFAYIYHQSFVVPQSISVEMIL | | | |
| DQILANL | | | |
| >29842.m003508 phosphatidate cytidylyltransferase, putative | | | |
| Length = 423 | | | |
| Score = 780 (279.6 bits), Expect = 1.4e−78, P = 1.4e−78 | | | |
| Identities = 152/385 (39%), Positives = 234/385 (60%) | | | |
| Sbjct: 18-399 = SEQ ID NO: 6 | | | |
| Query: | 36 | HKDASESVTP-VTKESTA--ATKESRKY-NFFIRTVWTFVMISGFFITLASGHAWCIVLI | 91 |
| | | H+ S V P KE+ + KY + +IRT T MI F + + GH + ++ | |
| Sbjct: | 18 | HRKRSNEVVPEAAKENGGHLLVDDQNKYKSMWIRTCSTVWMIGSFALIVYMGHLYITAMV | 77 |
| Query: | 92 | LGCQIATFKECIAVTSASGREKNLPLTKTLNWYLLFTTIYYLDGKSLFKFFQATF----Y | 147 |
| | | + QI KE + + +++LP + LNW+ FT + ++ G+ L + T + | |
| Sbjct: | 78 | VVIQIYMAKELFNLLRKAHEDRHLPGFRLLNWHFFFTAMLFVYGRILSQRLVNTVTSDKF | 137 |
| Query: | 148 | EYPVLNFIVTNHKFICYCLYLMGFVLFVCSLRKGFLKFQFGSLCVTHMVLLLVVFQAHLI | 207 |
| | | + ++N ++ H +CY LY+ GF+ F+ +L+K K+QFG THM+L++V Q+ | |
| Sbjct: | 138 | LFQLVNSLIKYHMAMCYFLYIAGFMWFILTLKKKMYKYQFGQYAWTHMILIVVFTQSSFT | 197 |
| Query: | 208 | IKNVLNGLFWFLLPCGLVIVNDIFAYLCGITFGKTKLIEISPKKTLEGFLGAWFFTALAS | 267 |
| | | + N+ G+FWFLLP L+++NDIFAY+CG FG+T LI++SPKKT EGF+GA T +++ | |
| Sbjct: | 198 | VANIFEGIFWFLLPASLIVINDIFAYICGFFFGRTPLIKLSPKKTWEGFIGASVTTMISA | 257 |
| Query: | 268 | IILTRILSPYTYLTCPVEDLHTNFFSNLTCELNPVFLPQVYRLPPIFFDKVQINSITVKP | 327 |
| | | +L ++ + +LTCP +DL + + L C+ P+F P+ + LP I++ P | |
| Sbjct: | 258 | FVLANMMGRFQWLTCPRKDLSSGW---LQCDPGPLFKPEYFILPEWVPQWFPWKEISILP | 314 |
| Query: | 328 | IYFHALNLATFASLFAPFGGFFASGLKRTFKVKDFGHSIPGHGGITDRVDCQFIMGSFAN | 387 |
| | | + +HAL L FAS+ APFGGFFASG KR FK+KDFG SIPGHGGITDR+DCQ +M FA | |
| Sbjct: | 315 | VQWHALWLGLFASIIAPFGGFFASGFKRAFKIKDFGDSIPGHGGITDRMDCQMVMAVFAY | 374 |
| Query: | 388 | LYYETFISEHRITVDTVLSTILMNL | 412 |
| | | +Y+++F+ I+V+ +L IL NL | |
| Sbjct: | 375 | IYHQSFVVPQSISVEMILDQILANL | 399 |
| * Cho2 | | | |
| SEQ ID NO: 7 Ricinus sp. 30131.m007049 (Cho2 homologue) | | | |
| EKNLTKFFEEVNGIKTEMEEITNLLLDLQDLNEDSKSTHSTKVLKGIRDRINSDMVTILRKAKIIKSRLESL | | | |
| >30131.m007049 syntaxin, arabidopsis thaliana, putative, | | | |
| Length = 296 | | | |
| Score = 74 (31.1 bits), Expect = 3.8, P = 0.98 | | | |
| Identities = 21/73 (28%), Positives = 36/73 (49%) | | | |
| Sbjct: 39-110 = SEQ ID NO: 7 | | | |
| Query: | 796 | EKDLTEFLTKVNVLKDGKFRPLGNKFFGMDSLKQLIKNSIGVELSSEYMRRVNGDAHVIS | 855 |
| | | EK+LT+F +VN +K + + N + L + K++ ++ R+N D I | |
| Sbjct: | 39 | EXNLTKFFEEVNGIKT-EMEEITNLLLDLQDLNEDSKSTHSTKVLKGIRDRINSDMVTIL | 97 |
| Query: | 856 | HRAWDIKQTLDSL | 868 |
| | | +A IK L+SL | |
| Sbjct: | 98 | RKAKIIKSRLESL | 110 |
| * Ckb1 | | | |
| A. SEQ ID NO: 8 Ricinus sp. 29709.m001188 (Ckb1 homologue) | | | |
| LVESAAEMLYGLIHVRYILTSKGMSAMLEKYKNYDFGRCPRVYCCGQPCLPVGQSDIPRSSTVKIYCPKCEDIYY | | | |
| PRSNVF | | | |
| >29709.m001188 casein kinase II beta chain, putative | | | |
| Length = 338 | | | |
| Score = 247 (92.0 bits), Expect = 1.6e−38, Sum P(2) = 1.6e−38 | | | |
| Identities = 43/81 (53%), Positives = 56/81 (69%) | | | |
| Sbjct: 118-236 = SEQ ID NO: 8 | | | |
| Query: | 118 | IIEHAAEQLYGLIHARFILTKPGLQAMAEKFDHKEFGTCPRYYCNGMQLLPCGLSDTVGK | 177 |
| | | ++E AAE LYGLIH R+ILT G+ AM EK+ + +FG CPR YC G LP G SD | |
| Sbjct: | 156 | LVESAAEMLYGLIHVRYILTSKGMSAMLEKYKNYDFGRCPRVYCCGQPCLPVGQSDIPRS | 215 |
| Query: | 178 | HTVRLYCPSCQDLYLPQSSRF | 198 |
| | | TV++YCP C+D+Y P+S+F | |
| Sbjct: | 216 | STVKIYCPKCEDIYYPRSNVF | 236 |
| B. SEQ ID NO: 9 Ricinus sp. 29709.m001188 (Ckb1 homologue) | | | |
| DEESETDSEESDVSGSDGDDTSWISWFCNLRGNEFFCEVDDEYIQDDFNLCGLSSQVPYYDYALDLILDVES | | | |
| >29709.m001188 Score = 171 (65.3 bits), Expect = 1.6e−38, Sum | | | |
| P(2) = 1.6e−38 | | | |
| Identities = 33/72 (45%), Positives = 50/72 (69%) | | | |
| Sbjct: 72-143 = SEQ ID NO: 9 | | | |
| Query: | 7 | EDYSRTGSSDDEDSGAYDE---WIPSFCSRFGHEYFCQVPTEFIEDDFNMTSLSQEVPHY | 63 |
| | | ++ S T S + + SG+ + WI FC+ G+E+FC+V E+I+DDFN+ LS +VP+Y | |
| Sbjct: | 72 | DEESETDSEESDVSGSDGDDTSWISWFCNLRGNEFFCEVDDEYIQDDFNLCGLSSQVPYY | 131 |
| Query: | 64 | RKALDLILDLEA | 75 |
| | | ALDLILD+E+ | |
| Sbjct: | 132 | DYALDLILDVES | 143 |
| * Ino2 | | | |
| SEQ ID NO: 10 Ricinus sp. 29669.m000818 (Ino2 homologue) | | | |
| HITPSMDSHLLANNRFIEEIPQSSWSSVPTQASEPDKTTDSEENSSQHQPLFKGQDH | | | |
| >29669.m000818 conserved hypothetical protein | | | |
| Length = 87 | | | |
| Score = 62 (26.9 bits), Expect = 0.18, P = 0.16 | | | |
| Identities = 18/59 (30%), Positives = 24/59 (40%) | | | |
| Sbjct: 27-83 = SEQ ID NO: 10 | | | |
| Query: | 153 | HIRSPKKQHRYTELNQRYPETHPHSNTGELPTNTA--DVPTEFTTREGPHQPI--GNDH | 207 |
| | | HI H N R+ E P S+ +PT + D T+ HQP+ G DH | |
| Sbjct: | 27 | HITPSMDSHLLA--NNRFIEEIPQSSWSSVPTQASEPDKTTDSEENSSQHQPLFKGQDH | 83 |
| * Mrps35 | | | |
| SEQ ID NO: 11 Ricinus sp. 28355.m000102 (Mrps35 homologue) | | | |
| PKLDEKVSCEYRELQEPVKIPGCVPIHGNKLLDPVQDRKNDAYKWFLHHSKRYKLADGIMVNSFTDLEGGAIKAL | | | |
| QEEEPAGKPPVYPVGPLVNMGSSSSREGAECLRWLDEQPHGSVLYVSFGSGGTLSYDQINELALGLEMSEQRFLW | | | |
| VARSPNDGVANA | | | |
| >28355.m000102 UDP-glucosyltransferase, putative | | | |
| Length = 426 | | | |
| Score = 79 (32.9 bits), Expect = 0.60, P = 0.45 | | | |
| Identities = 36/171 (21%), Positives = 75/171 (43%) | | | |
| Sbjct: 132-293 = SEQ ID NO: 11 | | | |
| Query: | 90 | PDLERGQSLEHPVTKKPLQLRYDGTLGPPPVENKRLQNIFKDRLLQPFPSNPHCKTNYVL | 149 |
| | | P L+ S E+ ++P+++ G P+ +L + +DR + H Y L | |
| Sbjct: | 132 | PKLDEKVSCEYRELQEPVKIP-----GCVPIHGNKLLDPVQDRKNDAYKWFLHHSKRYKL | 186 |
| Query: | 150 | SPQLKQSIFEEITVEGLSAQQVSQKYGLKIPRVEAIVKLVSVENSWNRRNRVSSDLKTMD | 209 |
| | | + + + F ++ + A Q + G P V + LV++ +S +R + L+ +D | |
| Sbjct: | 187 | ADGIMVNSFTDLEGGAIKALQEEEPAGK--PPVYPVGPLVNMGSSSSREG--AECLRWLD | 242 |
| Query: | 210 | ETLYR--MFPVFDSDASFKRENLSEIPVPQKTLASRFLTIAESEPFGPVDA | 258 |
| | | E + ++ F S + + ++E+ + + RFL +A S G +A | |
| Sbjct: | 243 | EQPHGSVLYVSFGSGGTLSYDQINELALGLEMSEQRFLWVARSPNDGVANA | 293 |
| * Opi3 | | | |
| SEQ ID NO: 12 Ricinus sp. 30131.m006852 (Opi3 homologue) | | | |
| LFGFGQFLNVRVYRLLGESGTYYGVRFGKSIPWVTEFPFGVIRDPQYVGSILSLL | | | |
| >30131.m006852 conserved hypothetical protein | | | |
| Length = 164 | | | |
| Score = 103 (41.3 bits), Expect = 3.6e−05, P = 3.6e−05 | | | |
| Identities = 27/56 (48%), Positives = 31/56 (55%) | | | |
| Sbjct: 72-126 = SEQ ID NO: 12 | | | |
| Query: | 104 | LFGLGQVLVLSSMYKLGITGTYLGDYFGILMDERVTGFPFNVSNNPMYQGSTLSFL | 159 |
| | | LFG GQ L + LG +GTY G FG + VT FPF V +P Y GS LS L | |
| Sbjct: | 72 | LFGFGQFLNVRVYRLLGESGTYYGVRFGKSIPW-VTEFPFGVIRDPQYVGSILSLL | 126 |
| * Rtc2 | | | |
| A. SEQ ID NO: 13 Ricinus sp. 29250.m000234 (Rtc2 homologue) | | | |
| GFVSLVSWGVAEVPQIITNFRTKSSHGVSLLFLLTWVAGDVFNLVGCLLEPATLPTQFYTALLYTTSTIVLVLQG | | | |
| LYYD | | | |
| >29250.m000234 conserved hypothetical protein | | | |
| Length = 377 | | | |
| Score = 184 (69.8 bits), Expect = 2.8e−25, Sum P(2) = 2.8e−25 | | | |
| Identities = 33/79 (41%), Positives = 54/79 (68%) | | | |
| Sbjct: 37-115 = SEQ ID NO: 13 | | | |
| Query: | 18 | GSISICCWIVVFVPQIYENFRRQSAEGLSLLFIVLWLLGDIFNVMGAMMQNL-LPTMIIL | 76 |
| | | G +S+ W V VPQI NFR +S+ G+SLLF++ W+ GD+FN++G +++ LPT | |
| Sbjct: | 37 | GFVSLVSWGVAEVPQIITNFRTKSSHGVSLLFLLTWVAGDVFNLVGCLLEPATLPTQFYT | 96 |
| Query: | 77 | AAYYTLADLILLIQCMWYD | 95 |
| | | A YT + ++L++Q ++YD | |
| Sbjct: | 97 | ALLYTTSTIVLVLQGLYYD | 115 |
| B. SEQ ID NO: 14 Ricinus sp. 29250.m000234 (Rtc2 homologue) | | | |
| QWLGWLMAAIYMGGRIPQIWLNIKRGSVEGLNPLMFIFALVANLTYVLSIVVRTTEWESIKANMPWLLDAAVCVA | | | |
| LDFFIILQYVYY | | | |
| conserved hypothetical protein | | | |
| Score = 150 (57.9 bits), Expect = 2.8e−25, Sum P(2) = 2.8e−25 | | | |
| Identities = 32/87 (36%), Positives = 53/87 (60%) | | | |
| Sbjct: 269-355 = SEQ ID NO: 14 | | | |
| Query: | 209 | QILGYLSAILYLGSRIPQIVLNFKRKSCEGVSFLFFLFACLGNTSFIISVL--------- | 259 |
| | | Q LG+L A +Y+G RIPQI LN KR S EG++ L F+FA + N ++++S++ | |
| Sbjct: | 269 | QWLGWLMAAIYMGGRIPQIWLNIKRGSVEGLNPLMFIFALVANLTYVLSIVVRTTEWESI | 328 |
| Query: | 260 | --SASWLIGSAGTLLMDFTVFIQFFLY | 284 |
| | | + WL+ +A + +DF + +Q+ Y | |
| Sbjct: | 329 | KANMPWLLDAAVCVALDFFIILQYVYY | 355 |
| C. SEQ ID NO: 15 Ricinus sp. 29250.m000234 (Rtc2 homologue) | | | |
| GFVSLVSWGVAEVPQIITNFRTKSSHGVSLLFLLTWVAGDVFNLVGCL | | | |
| conserved hypothetical protein | | | |
| Score = 83 (34.3 bits), Expect = 0.14, P = 0.13 | | | |
| Identities = 17/48 (35%), Positives = 28/48 (58%) | | | |
| Sbjct: 37-84 = SEQ ID NO: 15 | | | |
| Query: | 212 | GYLSAILYLGSRIPQIVLNFKRKSCEGVSFLFFLFACLGNTSFIISVL | 259 |
| | | G++S + + + +PQI+ NF+ KS GVS LF L G+ ++ L | |
| Sbjct: | 37 | GFVSLVSWGVAEVPQIITNFRTKSSHGVSLLFLLTWVAGDVFNLVGCL | 84 |
| D. SEQ ID NO: 16 Ricinus sp. 29250.m000234 (Rtc2 homologue) | | | |
| IPQIWLNIKRGSVEGLNPLMFIFALVANLTYVLSIVVRTTEWESIKANMPWLLDAAVCVALDFFIILQYVYYRYF | | | |
| REKK | | | |
| conserved hypothetical protein | | | |
| Score = 72 (30.4 bits), Expect = 2.4, P = 0.91 | | | |
| Identities = 21/79 (26%), Positives = 43/79 (54%) | | | |
| Sbjct: 284-362 = SEQ ID NO: 16 | | | |
| Query: | 30 | VPQIYENFRRQSAEGLSLLFIVLWLLGDIFNVMGAMM-----QNLLPTM--IILAAYYTL | 82 |
| | | +PQI+ N +R S EGL+ L + L+ ++ V+ ++ +++ M ++ AA | |
| Sbjct: | 284 | IPQIWLNIKRGSVEGLNPLMFIFALVANLTYVLSIVVRTTEWESIKANMPWLLDAAVCVA | 343 |
| Query: | 83 | ADLILLIQCMWYD--KEKK | 99 |
| | | D +++Q ++Y +EKK | |
| Sbjct: | 344 | LDFFIILQYVYYRYFREKK | 362 |
| *Scs2 | | | |
| SEQ ID NO: 17 Ricinus sp. 30174.m008835 (Scs2 homologue) | | | |
| LNIQPSELKFPFELKKQSSCSMQLTNKSNSYVAFKVKTTNPKKYCVRPNTGIILPGTACNVTVTMQAQKEAPPDM | | | |
| QCKDKFLLQSVAAPDGVTTKDITADTFTKEDGKVIEEFKLRVVYIPANPPSPVPEEPEEGLSPRSSVLENGDQDS | | | |
| SLLEAVSRSLEEPKE | | | |
| >30174.m008835 vesicle-associated membrane protein, putative | | | |
| Length = 238 | | | |
| Score = 200 (75.5 bits), Expect = 4.0e−17, P = 4.0e−17 | | | |
| Identities = 55/171 (32%), Positives = 85/171 (49%) | | | |
| Sbjct: 7-171 = SEQ ID NO: 17 | | | |
| Query: | 4 | VEISPDVLVYKSPLTEQSTEYASISNNSDQTIAFKVKTTAPKFYCVRPNAAVVAPGETIQ | 63 |
| | | + I P L + L +QS+ ++N S+ +AFKVKTT PK YCVRPN ++ PG | |
| Sbjct: | 7 | LNIQPSELKFPFELKKQSSCSMQLTNKSNSYVAFKVKTTNPKKYCVRPNTGIILPGTACN | 66 |
| Query: | 64 | VQVIFLGLTEEPAADFKCRDKFLVITLPSPYDLNGKAVADVWSDLEAEFKQQAISK-KIK | 122 |
| | | V V E P D +C+DKFL+ ++ +P +G D+ +D + + I + K++ | |
| Sbjct: | 67 | VTVTMQAQKEAPP-DMQCKDKFLLQSVAAP---DGVTTKDITADTFTKEDGKVIEEFKLR | 122 |
| Query: | 123 | VKYLIS--PDVHPAQ-NQNIQENKETVEPVVQDSEPKEVPAVVNEKEVPAE | 170 |
| | | V Y+ + P P + + + +E QDS E AV E P E | |
| Sbjct: | 123 | VVYIPANPPSPVPEEPEEGLSPRSSVLENGDQDSSLLE--AVSRSLEEPKE | 171 |

3. Glycine Sp./Soybean (“Sbjct”) Vs S. cerevisiae (“Query”) Alignments:

### * Cds1

### From Phytozome:

HSP#1 Subject 41076229-41076035 = SEQ ID NO: 18
SEQ ID NO: 18 Glycine sp.(Cds1 homologue)
PIIPGTSENHNLRPIICCLMQFSWKEISILPIQWHSLCLGLFASIIAPF
GGFFASGFKRAFKIK
HSP#2 Subject 41075939-41075877 = SEQ ID NO: 19
SEQ ID NO: 19 Glycine sp.(Cds1 homologue)
LQDFGDSIPGHGGITDRMDCQ
HSP#3 Subject 41077510-41077325 = SEQ ID NO: 20
SEQ ID NO: 20 Glycine sp.(Cds1 homologue)
LDLLYRFLLPATLIVINDIAAYIFGFFFGRTPLIKLSPKKTWEGFIGAS
VTTIISAFMVSRI
HSP#4 Subject 41078414-41078229 = SEQ ID NO: 21
SEQ ID NO: 21 Glycine sp.(Cds1 homologue)
CYCFGYLPFYWLSAGFMWFILTLKKKMYKYQFGQYAWTHMILIVVF
GQSSFTVASIFEGIFW

### * Ckb1

### From Phytozome:

Feature#1/HSP#1 Subject 5095580-5095903 = SEQ ID
NO: 22
SEQ ID NO: 22 Glycine sp.(Ckb1 homologue)
LIESAAEMLYGLIHARYVLTSKGMAAMVSFLLVDHSFCSLSSWRLALLVT
SLELLQLDKYKNYDFGRCPRVYCSGQPCLPVGQSDIPRSSTVKIYCPRC
EDLYYPRS
Feature#1/HSP#3 Subject 5095083-5095259 = SEQ ID
NO: 23
SEQ ID NO: 23 Glycine sp.(Ckb1 homologue)
SGSDGDDTSWISWFCNLRGNEFFCEVDDDYIQDDFNLCGLSSQVPYYDY
ALDLILDVES
Feature#2/HSP#2 Subject 5060232-5060552 = SEQ ID
NO: 24
SEQ ID NO: 24 Glycine sp.(Ckb1 homologue)
LIESAAEMLYGLIHARYILTSKGMAAMVFFSLLSCFLILYCGQNWHLMTL
DYLQLHKYKNYNFGRCPRVFCSGQPCLPVGQSDVPRSSTVKIYCPRCE
Feature#2/HSP#4 Subject 5059804-5059953 = SEQ ID
NO: 25
SEQ ID NO: 25 Glycine sp.(Ckb1 homologue)
WISWFCNLRGNEFFCEVDDDFVQDDFNLCGLSSQVPYYDYALDLILDVES

### From NCBI:

SEQ ID NO: 26 Glycine sp. ACU23831.1
(Ckb1 homologue)
SGSDGDDTSWISWFCNLRGNEFFCEVDGDYIQDDFNLCGLSSQVPYYDYA
LDLILDVESSHGDMFTEEQNELIESAAEMLYGLIHTRYVLTSKGMAAM
LDKYKNYDFGRCPRVYCSGQPCLPVGQSDIPRSSTVKIYCPRCEDLYYPR
SKYQGNIDGAYFGTTFPHLFLMTYGQLKPQKPAQGYVPRVFGFKVH

>gb|ACU23831.1| unknown [Glycine max]

### Length=261

Score=171 bits (434), Expect=8e-44, Method: Compositional matrix adjust.

### Identities=92/236 (39%), Positives=136/236 (58%), Gaps=46/236 (19%)

| Sbjct: 66-259 = SEQ ID NO: 26 | | | |
|---|---|---|---|
| Query | 12 | TGSSDDEDSGAYDEWIPSFCSRFGHEYFCQVPTEFIEDDFNMTSLSQEVPHYRKALDLIL | 71 |
| | | +GS D+ S WI FC+ G+E+FC+V ++I+DDFN+ LS +VP+Y ALDLIL | |
| Sbjct | 66 | SGSDGDDTS-----WISWFCNLRGNEFFCEVDGDYIQDDFNLCGLSSQVPYYDYALDLIL | 120 |
| Query | 72 | DLEA----MSDEEEDEDDVVEEDEVDQEMQSNDGHDEGKRRNKSPVVNKSIIEHAAEQLY | 127 |
| | | D+E+ M EE++E +IE AAE LY | |
| Sbjct | 121 | DVESSHGDMFTEEQNE----------------------------------LIESAAEMLY | 146 |
| Query | 128 | GLIHARFILTKPGLQAMAEKFDHKEFGTCPRYYCNGMQLLPCGLSDTVGKHTVRLYCPSC | 187 |
| | | GLIH R++LT G+ AM +K+ + +FG CPR YC+G LP G SD TV++YCP C | |
| Sbjct | 147 | GLIHTRYVLTSKGMAAMLDKYKNYDFGRCPRVYCSGQPCLPVGQSDIPRSSTVKIYCPRC | 206 |
| Query | 188 | QDLYLPQSSRFLCLEGAFWGTSFPGVFLKHFKELEEYVERKSKESYELKVFGFRIN | 243 |
| | | +DLY P+S ++GA++GT+FP +FL + +L+ +K + Y +VFGF+++ | |
| Sbjct | 207 | EDLYYPRSKYQGNIDGAYFGTTFPHLFLMTYGQLK---PQKPAQGYVPRVFGFKVH | 259 |
| Prodom search prediction | | | |
| SEQ ID NO: 27* Glycine sp. PD003829 (KINASE II BETA CASEIN SUBUNIT CK | | | |
| PHOSPHORYLATION PATHWAY SIGNALING WNT) | | | |
| ISWFCNLRGNEFFCEVDDEYIQDDFNLCGLSSQVPYYDYALDLILDVESSHGDMFTEEQNELVESAAEMLYGLIH | | | |
| VRYILTSKGMAAMLEKYKNYDFGRCPRVYCCGQPCLPVGQSDIPRSSTVKIYCPKCEDIYYPRSKYQGNIDGAYF | | | |
| GTTFPHLFLMTYGHLKPQKSTQNYVPRVFGFKLH | | | |
| >PD003829 (Closest domain: Q8LPD2_TOBAC 95-278) | | | |
| Number of domains in family: 137 | | | |
| Commentary (automatic): | | | |
| KINASE II BETA CASEIN SUBUNIT CK PHOSPHORYLATION PATHWAY | | | |
| SIGNALING WNT | | | |
| Length = 184 | | | |
| Score = 940 (366.7 bits), Expect = 6e−101 | | | |
| Identities = 170/184 (92%), Positives = 177/184 (96%) | | | |
| Sbjct: 95-278 = SEQ ID NO: 27 | | | |
| Query: | 76 | ISWFCNLRGNEFFCEVDGDYIQDDFNLCGLSSQVPYYDYALDLILDVESSHGDMFTEEQN | 135 |
| | | ISWFCNLRGNEFFCEVD +YIQDDFNLCGLSSQVPYYDYALDLILDVESSHGDMFTEEQN | |
| Sbjct: | 95 | ISWFCNLRGNEFFCEVDDEYIQDDFNLCGLSSQVPYYDYALDLILDVESSHGDMFTEEQN | 154 |
| Query: | 136 | ELIESAAEMLYGLIHTRYVLTSKGMAAMLDKYKNYDFGRCPRVYCSGQPCLPVGQSDIPR | 195 |
| | | EL+ESAAEMLYGLIH RY+LTSKGMAAML+KYKNYDFGRCPRVYC GQPCLPVGQSDIPR | |
| Sbjct: | 155 | ELVESAAEMLYGLIHVRYILTSKGMAAMLEKYKNYDFGRCPRVYCCGQPCLPVGQSDIPR | 214 |
| Query: | 196 | SSTVKIYCPRCEDLYYPRSKYQGNIDGAYFGTTFPHLFLMTYGQLKPQKPAQGYVPRVFG | 255 |
| | | SSTVKIYCP+CED+YYPRSKYQGNIDGAYFGTTFPHLFLMTYG LKPQK Q YVPRVFG | |
| Sbjct: | 215 | SSTVKIYCPKCEDIYYPRSKYQGNIDGAYFGTTFPHLFLMTYGHLKPQKSTQNYVPRVFG | 274 |
| Query: | 256 | FKVH | 259 |
| | | FK+H | |
| Sbjct: | 275 | FKLH | 278 |

NCBI:
SEQ ID NO: 28* Glycine sp. ACO91805.1 (phospholipid N methyltransferase)
PLIAFGQFLNFRVYQLLGETGTYYGVRFGETI-PWVTEFPFGVIKDPQYVGSIMSILA

>gb|ACO91805.1| phospholipid N-methyltransferase [Glycine max] GENE ID: 100301902 PLMT| phospholipid N-methyltransferase [Glycine max
(10 or fewer PubMed links)
Score=38.1 bits (87), Expect=8e-04, Method: Compositional matrix adjust.

### Identities=25/58 (43%), Positives=28/58 (48%), Gaps=1/58 (2%)

| Sbjct: 74-130 = SEQ ID NO: 28 | | | |
|---|---|---|---|
| Query | 103 | ALFGLGQVLVLSSMYKLGITGTYLGDYFGILMDERVTGFPFNVSNNPMYQGSTLSFLG | 160 |
| | | L GQ L LG TGTY G FG + VT FPF V +P Y GS +S L | |
| Sbjct | 74 | PLIAFGQFLNFRVYQLLGETGTYYGVRFGKTI-PWVTEFPFGVIKDPQYVGSIMSILA | 130 |

* Rtc2
Phytozome:
Gm03 subject 35049077-35048961 = SEQ ID NO: 29
SEQ ID NO: 29 Glycine sp. (Rtc2 homologue)
GLISVIVWVVAEIPQILTNYRTKSAEGLSVTFLITWIIG
Gm19 subject 37947353-37947237 = SEQ ID NO: 30
SEQ ID NO: 30 Glycine sp. (Rtc2 homologue)
GLINVIVWVVAEIPQIIPNYRTKSAEGLSVTFLVTWIIG
Gm11 subject 4783865-4784035 = SEQ ID NO: 31
SEQ ID NO: 31 Glycine sp. (Rtc2 homologue)
GFISLICWGVAEIPQIITNFRAKSSHGVSLAFLLTWVAGLVSLSTFLHFLIVLSKSY
Gm01 subject 50607082-50606966 = SEQ ID NO: 32
SEQ ID NO: 32 Glycine sp. (Rtc2 homologue)
GFISLVCWGVAEIPQIITNFRAKSSHGVSLAFLLTWVAG

### NCBI:

gb|ACU24122.1| unknown [Glycine max]

### Length=379

A. SEQ ID NO: 33 Glycine sp. ACU24122.1
(Rtc2 homologue)
GLTSLVFWGVAEIPQIITIFRTKKSHGVSLVFLLTWVAGDICNLTGCILE
PATLPTQYYTALLYTITTIVLLLLIVYYDYISRWYKHRQKVNLVRDH

Score=57.8 bits (138), Expect=2e-09, Method: Compositional matrix adjust.

### Identities=32/97 (33%), Positives=53/97 (55%), Gaps=1/97 (1%)

| Sbjct: 37-133 = SEQ ID NO: 33 | | | |
|---|---|---|---|
| Query | 18 | GSISICCWIVVFVPQIYENFRRQSAEGLSLLFIVLWLLGDIFNVMGAMMQ-NLLPTMIIL | 76 |
| | | G S+ W V +PQI FR + + G+SL+F++ W+ GDI N+ G +++ LPT | |
| Sbjct | 37 | GLTSLVFWGVAEIPQIITIFRTKKSHGVSLVFLLTWVAGDICNLTGCILEPATLPTQYYT | 96 |
| Query | 77 | AAYYTLADLILLIQCMWYDKEKKSILQEVKKNVDPVH | 113 |
| | | A YT+ ++LL+ ++YD + K N+ H | |
| Sbjct | 97 | ALLYTITTIVLLLLIVYYDYISRWYKHRQKVNLVRDH | 133 |

B. SEQ ID NO: 34 Glycine sp. ACU24122.1 (Rtc2 homologue)
YEKHSTFGQWLGWLMAAIYISGRVPQIWLNIKRSSVEGLNPFMFVFALVANVTYVGSIL

Score=53.1 bits (126), Expect=5e-08, Method: Compositional matrix adjust.

### Identities=24/59 (41%), Positives=39/59 (66%), Gaps=0/59 (0%)

| Sbjct: 264-322 = SEQ ID NO: 34 | | | |
|---|---|---|---|
| Query | 201 | FEQINLPAQILGYLSAILYLGSRIPQIVLNFKRKSCEGVSFLFFLFACLGNTSFIISVL | 259 |
| | | +E+ + Q LG+L A +Y+ R+PQI LN KR S EG++ F+FA + N +++ S+L | |
| Sbjct | 264 | YEKHSTFGQWLGWLMAAIYISGRVPQIWLNIKRSSVEGLNPFMFVFALVANVTYVGSIL | 322 |

C. SEQ ID NO: 35 Glycine sp. ACU24122.1 (Rtc2 homologue)
LTSLVFWGVAEIPQIITIFRTKKSHGVSLVFLLTWVAGD

Score=28.9 bits (63), Expect=0.85, Method: Compositional matrix adjust.

### Identities=14/39 (36%), Positives=24/39 (62%), Gaps=1/39 (3%)

| Sbjct: 38-76 = SEQ ID NO: 35 | | | |
|---|---|---|---|
| Query | 214 | LSAILYLG-SRIPQIVLNFKRKSCEGVSFLFFLFACLGN | 251 |
| | | L+++++ G + IPQI+ F+ K GVS +F L G+ | |
| Sbjct | 38 | LTSLVFWGVAEIPQIITIFRTKKSHGVSLVFLLTWVAGD | 76 |

D.SEQ ID NO: 36 Glycine sp. ACU24122.1 (Rtc2 homologue)
VPQIWLNIKRSSVEGLNPFMFVFALVANV

Score=27.7 bits (60), Expect=2.1, Method: Compositional matrix adjust.

### Identities=12/29 (41%), Positives=18/29 (62%), Gaps=0/29 (0%)

Sbjct: 287-315 = SEQ ID NO: 36

| | | | |
|---|---|---|---|
| Query | 30 | VPQIYENFRRQSAEGLSLLFIVLWLLGDI | 58 |
| | | VPQI+ N +R S EGL+ V L+ ++ | |
| Sbjct | 287 | VPQIWLNIKRSSVEGLNPFMFVFALVANV | 315 |

* Scs2
Gm01 Subject 44980635-44980751 = SEQ ID NO: 37
SEQ ID NO: 37 Glycine sp. (Scs2 homologue)
QFMVLQVKTTNPKKYCVRPNTGVVMPRSTCDVIGFFFSL
Gm05 Subject 3726981-3726889 = SEQ ID NO: 38
SEQ ID NO: 38 Glycine L sp. (Scs2 homologue)
CLFFKVKTTNPKKYCVRPNTGIVTPRSTCDV
Gm03 Subject 3637572-3637432 = SEQ ID NO: 39
SEQ ID NO: 39 Glycine sp. (Scs2 homologue)
TCNFDLGVHFMVLQVKTTNPKKYCVRPNTGVVMPRSTCDVIGFFFSL

Phytozome:

### NCBI:

SEQ ID NO: 40 Glycine sp. ACU21258.1
(Scs2 homologue)
LHIEPAELRFVFELKKQSSCLVQLANNTDHFLAFKVKTTSPKKYCVRPN
IGIIKPNDKCDFTVTMQAQRMAPPDMLCKDKFLIQSTVVPVGTTEDDIT
SDMFAKDSGKFIEEKKLRVVLISP

>gb|ACU21258.1| unknown [Glycine max]

### Length=295

Score=73.6 bits (179), Expect=2e-14, Method: Compositional matrix adjust.

### Identities=47/127 (37%), Positives=68/127 (54%), Gaps=6/127 (5%)

| Sbjct: 6-127 = SEQ ID NO: 40 | | | |
|---|---|---|---|
| Query | 4 | VEISPDVLVYKSPLTEQSTEYASISNNSDQTIAFKVKTTAPKFYCVRPNAAVVAPGETIQ | 63 |
| | | + I P L + L +QS+ ++NN+D +AFKVKTT+PK YCVRPN ++ P + | |
| Sbjct | 6 | LHIEPAELRFVFELKKQSSCLVQLANNTDHFLAFKVKTTSPKKYCVRPNIGIIKPNDKCD | 65 |
| Query | 64 | VQVIFLGLTEEPAADFKCRDKFLVITLPSPYDLNGKAVADVWSDLEAEFKQQAI-SKKIK | 122 |
| | | V P D C+DKFL+ + P G D+ SD+ A+ + I KK++ | |
| Sbjct | 66 | FTVTMQAQRMAP-PDMLCKDKFLIQSTVVPV---GTTEDDITSDMFAKDSGKFIEEKKLR | 121 |
| Query | 123 | VKYLISP | 129 |
| | | V LISP | |
| Sbjct | 122 | V-VLISP | 127 |

### Example 4

### Knockdown of Gene Homologues in Arabidopsis thaliana

As noted in the Examples above, genes were identified in Saccharomyces and Chlamydomonas which, when knocked down, were observed to increase lipid droplet accumulation and the production of triacylglycerols with saturated fatty acid chains. Homologues of these genes were identified in the plant Arabidopsis (a model plant and relative of the Brassicas), and mutant Arabidopsis plants were generated in which at least one of the identified gene homologues was knocked down.

Arabidopsis mutants were obtained from a commercial service (NASC—The European Arabidopsis Stock Centre). The Arabidopsis mutants were generated by T-DNA Insertional Mutagenesis, and a description of this methodology is provided in O'Malley and Ecker, (2010), “Linking genotype to phenotype using the Arabidopsis unimutant collection”, The Plant Journal, 61, 928-940 (the contents of which are incorporated herein by reference in their entirety).

The Arabidopsis mutants are single, segregating flank-tagged T3-generation transferred DNA (T-DNA) lines and were generated by vacuum infiltration of Columbia (Col) plants (Columbia-0; CS60000, the sequenced genome) with Agrobacterium tumefaciens vector pROK2, a derivative of the pBIN19 vector (see Baulcombe et al. (1986), “Expression of biologically-active viral satellite RNA from the nuclear genome of transformed plants”, Nature, 321(6068):446-449).

T-DNA transformed plants were grown, genomic DNA prepared, and T-DNA flanking plant DNA was recovered and sequenced. Insertion site sequences were aligned with the Arabidopsis genome sequence and gene annotation added. Kanamycin (NTPII marker) was employed for selection of plants having T-DNA insertions. The presence of the T-DNA insertion in the gene was determined by PCR using primers from the T-DNA Left-border and the target gene sequence.

For PCR 1, the LBa1 primer was used: 5′ tggttcacgtagtgggccatcg 3′ (SEQ ID NO: 42). For PCR 2 and sequencing, the LBb1 primer was used: 5′ gcgtggaccgcttgctgcaact 3′ (SEQ ID NO: 43). Separate Arabidopsis mutants were generated each having one of the following genes knocked out by T-DNA insertion: CDS1, CDS2, RTC2, CKb1 or SCS2, a shown in Table 2 below.

TABLE 2

| | | TAIR search results | | | | | | |
|---|---|---|---|---|---|---|---|---|
| | | | Score | | | | | |
| Gene | Match to | | E | | Allele | | Pheno- | Seed |
| ID | Brassica | Best hits (locus name) | value | Germplasm name (other name) | Mutagen | Genotype | type | population |
| CDS1 | XA_0051 | AT1G62430 | 6e−83 | SALK_130379(N630379) | T-DNA insertion | heterozygous | NA | T2 and T3 |
| | Length = | CDS1, CDP- | | SALK_088268C(N657989) | = | homozygous | NA | NA |
| | 1633677 | DIACYLGLYCEROL | | | | | | |
| | E-value = | SYNTHASE 1 | | | | | | |
| | 1.37e−34 | Not found in NCBI | | | | | | |
| | | AT4G22340 | 5e−82 | SALK_079137(N579137) | = | heterozygous | NA | T2 and T3 |
| | | CDS2, | | SALK_148049(N648049) | = | heterozygous | NA | T2 and T3 |
| | | CYTIDINEDIPHOS- | | SALK_148057(N648057) | = | heterozygous | NA | T2 and T3 |
| | | PHATE | | SALK_099882C(N681519) | = | homozygous | NA | NA |
| | | DIACYLGLYCEROL | | SALK_106246C(N670013) | = | homozygous | NA | NA |
| | | SYNTHASE 2 | | | | | | |
| | | found in NCBI | | | | | | |
| RTC2 | XA_0011r | AT4G20100 | 1e−26 | SALK_128175(N628175) | = | NA | NA | T2 and T3 |
| | Length = | PQ-loop repeat family | | SALK_108796C (N682757) | = | homozygous | NA | NA |
| | 4370687 | protein/transmembrane | | | | | | |
| | E-value = | family protein | | | | | | |
| | 2.04e−04 | found in NCBI | | | | | | |
| CKb1 | XA_0048r | AT5G47080 | 3e−42 | SALK_140678 (N640678) | = | heterozygous | NA | T2 and T3 |
| | Length = | CKB1 CASEIN | | SALK_140689 (N640689) | = | heterozygous | NA | T2 and T3 |
| | 1689678 | KINASE II BETA | | SALK_030209C (N670492) | = | homozygous | NA | NA |
| | E-value = | CHAIN 1 | | SALK_092883C (N680004) | = | homozygous | NA | NA |
| | 1.15e−34 | found in NCBI | | | | | | |
| | & | | | | | | | |
| | XA_0048r | | | | | | | |
| | Length = | | | | | | | |
| | 1689678 | | | | | | | |
| | E-value = | | | | | | | |
| | 1.15e−34 | | | | | | | |
| SCS2 | >XA_0001r | AT1G08820 | 4e−12 | SAIL_278_A07/Stock: CS812847 | = | NA | NA | T1 |
| | Length = | VAMP/SYNAPTOBRE | | (SAIL_278_A07) | | | | |
| | 10813983 | VIN-ASSOCIATED | | | | | | |
| | E-value = | PROTEIN 27-2 | | | | | | |
| | 2.04e−08 | (VAP27-2) | | | | | | |
| | | Found in NCBI | | | | | | |

Table 2 shows the yeast gene id, the location on the Brassica rapa genome of the Brassica homolog (identified from the Arabidopsis homolog), the best Arabidopsis homologs (including their BLAST score), the name of the Arabidopsis T-DNA knock-out mutants for each Arabidopsis homolog, from the NASC/SALK mutant collection, whether the mutant is heterozygous or homozygous, a phenotype if available and the T-DNA generation of the seed.

Genomic sequences for each gene knocked out in each Arabidopsis thaliana mutant is indicated below:

(i) Arabidopsis thaliana CDS1—SEQ ID NO: 54
Arabidopsis thaliana phosphatidate cytidylyltransferase (CDS 1)
other names ATCDS1, CDP-DIACYLGLYCEROL SYNTHASE 1, CDS1, F24O1.17, F24O1_{—}17
encodes protein defined in SEQ ID NO: 49 (NCBI Reference Sequence: NM_{—}104923.3)
encoded protien is a CDP-diacylglycerol synthase, involved in phospholipid biosynthesis
mRNA sequence defined in SEQ ID NO: 44 (NCBI Reference Sequence NM_{—}104923.3)

(ii) Arabidopsis thaliana CDS2—SEQ ID NO: 45
cytidinediphosphate diacylglycerol synthase 2 (CDS2)
Sequence name AT4G22340.3
functions in phosphatidate cytidylyltransferase activity
located in endomembrane system, membrane
Tair Accession Sequence: 4010726215

(iii) Arabidopsis thaliana RTC2—SEQ ID NO: 46
Sequence name AT4G20100.1
PQ-loop repeat family protein/transmembrane family protein
Tair accession: Locus: 2119777

(iv) Arabidopsis thaliana Ckb1—SEQ ID NO: 47
sequence name AT5G47080.1
Tair Accession Sequence 4010729433
CASEIN KINASE II BETA CHAIN 1, CKB1, K14A3.3, K14A3_{—}3
Regulatory subunit beta of casein kinase II (CK2)

(v) Arabidopsis thaliana SCS2—SEQ ID NO: 48
Sequence name AT1G08820.1
Tair Accession Sequence: 1009087352
encodes VAP33-like protein that interacts with cowpea mosaic virus protein 60K. Is a SNARE-like protein that may be involved in vesicular transport to or from the ER.

Protein sequences identified in Brassica rapa encoded by genes homologous to those knocked out in Arabidopsis are indicated below:

(i) Brassica rapa CDS1: SEQ ID NO: 1

(ii) Brassica rapa Ckb1: SEQ ID NO: 2 and SEQ ID NO: 3

(iii) Brassica rapa Rtc2: SEQ ID NO: 4

(iv) Brassica rapa Scs2: SEQ ID NO: 5

Gene sequences (egnomic DNA) identified in Brassica rapa homologous to those knocked out in Arabidopsis are indicated below:
(i) Brassica rapa CDS1: SEQ ID NO: 50
   Brassica database (http://brassicadb.org)—gene i.d: Bra027046 positions 28523068 . . . 28527091 (+strand)
(ii) Brassica rapa Ckb1: SEQ ID NO: 51
   Brassica database (http://brassicadb.org)—gene i.d: Bra025515 positions 8451379 . . . 8451925 (−strand)
(iii) Brassica rapa Rtc2: SEQ ID NO: 52
   Brassica database (http://brassicadb.org)—gene i.d: Bra011711 positions 938214 . . . 940516 (+strand)
(iv) Brassica rapa Scs2: SEQ ID NO: 53
   Brassica database (http://brassicadb.org)—gene i.d: Bra000978 positions 17997049 . . . 17998305 (−strand)

Seeds derived from transformed plants were produced and will be analysed for lipid properties using standard methods. For example, oil bodies may be purified using the method described by Tzen et al. (see Tzen et al. (1997), “A new method for seed oil body purification and examination of oil body integrity following germination”, Journal of Biochemistry, 121, 762-768). Briefly, in a typical oil body purification, 100-200 mg of seeds can be soaked in Milli Q grade water for 1 h at room temperature, and subsequently ground 20 times for 15 s in 4 ml of 10 mM sodium phosphate buffer (pH 7.5) containing 0.6 M sucrose (buffer 1) with a Potter grinder driven by a Heidolph motor (rate 7). The sample may be cooled on ice between each grinding cycle, and the potter may be rinsed by 8 ml of buffer 1.

The suspension can be overlaid by one volume of 10 mM sodium phosphate buffer (pH 7.5) containing 0.4 M sucrose (buffer 2) and spun at 10,000×g and 4° C. for 30 min in a Kontron Ultracentrifuge equipped with a swinging-bucket rotor. The floating oil body fraction can be collected, resuspended in 12 ml of 5 mM sodium phosphate buffer (pH 7.5) containing 0.2 M sucrose and 0.1% (v/v) Tween 20. The suspension can then be overlaid by one volume of 10 mM sodium phosphate buffer (pH 7.5) and spun at 10,000×g and 4° C. for 30 min in a Kontron Ultracentrifuge equipped with a swinging-bucket rotor. The oil body fraction can be resuspended in 12 ml of buffer 1 additionally containing 2 M NaCl, overlaid by one volume of 10 mM sodium phosphate buffer containing 0.25 M sucrose and 2 M NaCl and spun. The oil body fraction can be collected (CF3), resuspended in 8 ml of 9 M urea and left on a shaker (60 rpm) at room temperature for 10 min. Then the suspension can be placed in centrifuge tubes, overlaid by one volume of 10 mM sodium phosphate buffer (pH 7.5) and spun. The oil body fraction can be collected (CF4), resuspended in 4 ml of buffer 1 and then mixed with 4 ml of hexane. After centrifugation and removal of the upper hexane layer, the oil body fraction can be collected and resuspended in 4 ml of buffer 1. In a last step, the resuspension can be overlaid by one volume of buffer 2 and centrifuged. The floating oil body fraction can be collected (CF6), resuspended in a minimal volume of buffer 1 and stored at 4° C. till further use.

Lipid anaylsis will be performed using standard techniques such as those described in Example 1 above. For example, thin layer chromatography (TLC) may be used (see, for example Fei et al. (2008), “Fld1p, a functional homologue of human seipin, regulates the size of lipid droplets in yeast”, J Cell Biol 180: 473-482; Low et al. (2008), “Caspase-dependent and -independent lipotoxic cell-death pathways in fission yeast”, J Cell Sci 121: 2671-2684).

It is envisaged that lipid analysis will reveal increased quantities/proportions of triacylglycerol (TAG) production, and in particular increased quantities/proportions of TAGs comprising saturated fatty acid chains.

What is claimed is:

1. A method for increasing triacylglycerol production in a cell, the method comprising modifying the cell to increase phosphatidic acid production.

2. The method according to claim 1, wherein said modifying comprises inhibiting the phosphatidylethanolamine N-methyltransferase (PEMT) pathway in said cell.

3. The method according to claim 2, wherein said inhibiting comprises:
(i) reducing the quantity or activity of an enzyme, enzyme cofactor, precursor compound, or intermediate compound of said PEMT pathway; or
(ii) reducing the quantity or activity of transcription factor that regulates a PEMT pathway protein.

4. The method according to claim 3, wherein the PEMT pathway enzyme is phosphatidylethanolamine methyltransferase or phospholipid methyltransferase.

5-6. (canceled)

7. The method according to claim 2, wherein said inhibiting comprises reducing the quantity or activity of a protein selected from the group consisting of: inositol-1-phosphate synthase, CDP-diacylglycerol synthase, casein kinase II, a beta regulatory subunit of casein kinase II, a protein homologous to Saccharomyces cerevisiae INO2/YDR123C, a protein homologous to Saccharomyces cerevisiae INO4/YOL108C, a protein homologous to Saccharomyces cerevisiae RTC2/YBR147W, a protein homologous to Saccharomyces cerevisiae MRPS35/YGR165W, and a protein homologous to Saccharomyces cerevisiae FLD1/YLR404W.

8. The method according to claim 1, wherein the cell is a bacterial cell, a yeast, an algal cell or a plant cell.

9. The method according to claim 1, wherein the triacylglycerol comprises at least one saturated fatty acid chain.

10. The method according to claim 1, wherein the triacylglycerol comprises at least one short chain fatty acid having less than 14 carbon atoms.

11. The method according to claim 1, wherein the method comprises inhibiting the expression of a gene in said cell.

12. A method for producing a genetically modified organism, the method comprising making at least one genetic modification to a cell of the organism that increases phosphatidic acid production in the cell, wherein triacylglycerol production in the cell is elevated compared to a corresponding wild-type cell, and said organism is bacterium, yeast, alga, or plant.

13. The method according to claim 12, wherein said genetic modification inhibits the phosphatidylethanolamine N-methyltransferase (PEMT) pathway in said cell.

14. The method according to claim 13, wherein said genetic modification:
(i) reduces the quantity or activity of an enzyme, enzyme cofactor, precursor compound, or intermediate compound of said PEMT pathway; or
(ii) reduces the quantity or activity of a transcription factor that regulates a PEMT pathway protein.

15. The method according to claim 13, wherein the PEMT pathway enzyme is phosphatidylethanolamine methyltransferase or phospholipid methyltransferase.

16-17. (canceled)

18. The method according to claim 12, wherein said genetic modification reduces the quantity or activity of a protein selected from the group consisting of: inositol-1-phosphate synthase, CDP-diacylglycerol synthase, casein kinase II, a beta regulatory subunit of casein kinase II, a protein homologous to Saccharomyces cerevisiae INO2/YDR123C, a protein homologous to Saccharomyces cerevisiae INO4/YOL108C, a protein homologous to Saccharomyces cerevisiae RTC2/YBR147W, a protein homologous to Saccharomyces cerevisiae MRPS35/YGR165W, a protein homologous to Saccharomyces cerevisiae FLD1/YLR404W, and a protein homologous to Saccharomyces cerevisiae FLD1/YLR404W.

19. (canceled)

20. The method according to claim 12, wherein said genetic modification inhibits the expression of a gene in said cell.

21. A method for producing a biofuel, the method comprising:
cultivating a genetically modified organism produced according to claim 12,
isolating triacylglycerols produced by the organism; and
transesterifying the triacylglycerols to produce the biofuel.

22-23. (canceled)

24. The method according to claim 7, wherein the protein comprises a sequence as set forth in any one of SEQ ID NOs 1-36, SEQ ID NO: 49, or a variant of any one of said sequences, or a fragment of any one of said sequences.

25. The method according to claim 11, wherein said gene comprises a sequence as set forth in any one of SEQ ID NOs 44-47, SEQ ID NOs 50-52, SEQ ID NO: 54, a variant of any one of said sequences, or a fragment of any one of said sequences.

26. The method according to claim 18, wherein the protein comprises a sequence as set forth in any one of SEQ ID NOs 1-36, SEQ ID NO: 49, or a variant of any one of said sequences, or a fragment of any one of said sequences.

27. The method according to claim 20, wherein said gene comprises a sequence as set forth in any one of SEQ ID NOs 44-47, SEQ ID NOs 50-52, SEQ ID NO: 54, a variant of any one of said sequences, or a fragment of any one of said sequences.


SEQUENCE LISTING (54 sequences)

SEQ ID NO: 1 - protein, 209 aa, Brassica sp.
DKNKYRSMWIRTCSSLWMLGGVFFIIYMGHLYIWAMVVGIQIFMAKELFFLLRRAHEERRLPGFRLLNWHFFFTAMLFVYGRILQQQLVNTVSSDRFIYKLVSGLIKYQMVICYFLYIAGFMWFILTLKKKMYKYQFGQYAWTHMILIVVFTQSSFTVANIFEGIFWFLLPAALIAMNDVAAYFFGFYFGKTPLIKLSPKKTWEGFIGA

SEQ ID NO: 2 - protein, 64 aa, Brassica sp.
SEVSGSDEEDLAWTTWFCKLPGNEFLCEVDDCFILDNFNLCGLRHQVPFYDNALDLILDDDSSS

SEQ ID NO: 3 - protein, 151 aa, Brassica sp.
LIESAAEMLYGLIHARYILTDKGFLSMLNKYNKSEFGRCPRVYCSGQSCLPIGLSDVPGASTVKIYCPKCEDIYHQPSKYQGSSSILLLVRIRYFSHLCVLVFFIHIKTDIDGSYFGTAFPHLFLMYYPSRRPKKVSSQSYVPRVFGFNLH

SEQ ID NO: 4 - protein, 45 aa, Brassica sp.
GIVSLICWGVAEIPQIITNFRTKSSHGVSLSFLLAWVAGSVHSII

SEQ ID NO: 5 - protein, 104 aa, Brassica sp.
KVELKKQSSCSLQISNKTSTQVVAFKVKTTNPRKYCVRPNTGVVLPGDSCNVTGDQLNCVFLFLFKNIYYTDQLNYVCLFLTLVTMQAQKEAPLDMQCKDKFLV

SEQ ID NO: 6 - protein, 382 aa, Ricinus sp.
HRKRSNEVVPEAAKENGGHLLVDDQNKYKSMWIRTCSTVWMIGSFALIVYMGHLYITAMVVVIQIYMAKELFNLLRKAHEDRHLPGFRLLNWHFFFTAMLFVYGRILSQRLVNTVTSDKFLFQLVNSLIKYHMAMCYFLYIAGFMWFILTLKKKMYKYQFGQYAWTHMILIVVFTQSSFTVANIFEGIFWFLLPASLIVINDIFAYICGFFFGRTPLIKLSPKKTWEGFIGASVTTMISAFVLANMMGRFQWLTCPRKDLSSGWLQCDPGPLFKPEYFILPEWVPQWFPWKEISILPVQWHALWLGLFASIIAPFGGFFASGFKRAFKIKDFGDSIPGHGGITDRMDCQMVMAVFAYIYHQSFVVPQSISVEMILDQILANL

SEQ ID NO: 7 - protein, 72 aa, Ricinus sp.
EKNLTKFFEEVNGIKTEMEEITNLLLDLQDLNEDSKSTHSTKVLKGIRDRINSDMVTILRKAKIIKSRLESL

SEQ ID NO: 8 - protein, 81 aa, Ricinus sp.
LVESAAEMLYGLIHVRYILTSKGMSAMLEKYKNYDFGRCPRVYCCGQPCLPVGQSDIPRSSTVKIYCPKCEDIYYPRSNVF

SEQ ID NO: 9 - protein, 72 aa, Ricinus sp.
DEESETDSEESDVSGSDGDDTSWISWFCNLRGNEFFCEVDDEYIQDDFNLCGLSSQVPYYDYALDLILDVES

SEQ ID NO: 10 - protein, 57 aa, Ricinus sp.
HITPSMDSHLLANNRFIEEIPQSSWSSVPTQASEPDKTTDSEENSSQHQPLFKGQDH

SEQ ID NO: 11 - protein, 162 aa, Ricinus sp.
PKLDEKVSCEYRELQEPVKIPGCVPIHGNKLLDPVQDRKNDAYKWFLHHSKRYKLADGIMVNSFTDLEGGAIKALQEEEPAGKPPVYPVGPLVNMGSSSSREGAECLRWLDEQPHGSVLYVSFGSGGTLSYDQINELALGLEMSEQRFLWVARSPNDGVANA

SEQ ID NO: 12 - protein, 55 aa, Ricinus sp.
LFGFGQFLNVRVYRLLGESGTYYGVRFGKSIPWVTEFPFGVIRDPQYVGSILSLL

SEQ ID NO: 13 - protein, 79 aa, Ricinus sp.
GFVSLVSWGVAEVPQIITNFRTKSSHGVSLLFLLTWVAGDVFNLVGCLLEPATLPTQFYTALLYTTSTIVLVLQGLYYD

SEQ ID NO: 14 - protein, 87 aa, Ricinus sp.
QWLGWLMAAIYMGGRIPQIWLNIKRGSVEGLNPLMFIFALVANLTYVLSIVVRTTEWESIKANMPWLLDAAVCVALDFFIILQYVYY

SEQ ID NO: 15 - protein, 48 aa, Ricinus sp.
GFVSLVSWGVAEVPQIITNFRTKSSHGVSLLFLLTWVAGDVFNLVGCL

SEQ ID NO: 16 - protein, 79 aa, Ricinus sp.
IPQIWLNIKRGSVEGLNPLMFIFALVANLTYVLSIVVRTTEWESIKANMPWLLDAAVCVALDFFIILQYVYYRYFREKK

SEQ ID NO: 17 - protein, 165 aa, Ricinus sp.
LNIQPSELKFPFELKKQSSCSMQLTNKSNSYVAFKVKTTNPKKYCVRPNTGIILPGTACNVTVTMQAQKEAPPDMQCKDKFLLQSVAAPDGVTTKDITADTFTKEDGKVIEEFKLRVVYIPANPPSPVPEEPEEGLSPRSSVLENGDQDSSLLEAVSRSLEEPKE

SEQ ID NO: 18 - protein, 64 aa, Glycine sp.
PIIPGTSENHNLRPIICCLMQFSWKEISILPIQWHSLCLGLFASIIAPFGGFFASGFKRAFKIK

SEQ ID NO: 19 - protein, 21 aa, Glycine sp.
LQDFGDSIPGHGGITDRMDCQ

SEQ ID NO: 20 - protein, 62 aa, Glycine sp.
LDLLYRFLLPATLIVINDIAAYIFGFFFGRTPLIKLSPKKTWEGFIGASVTTIISAFMVSRI

SEQ ID NO: 21 - protein, 62 aa, Glycine sp.
CYCFGYLPFYWLSAGFMWFILTLKKKMYKYQFGQYAWTHMILIVVFGQSSFTVASIFEGIFW

SEQ ID NO: 22 - protein, 107 aa, Glycine sp.
LIESAAEMLYGLIHARYVLTSKGMAAMVSFLLVDHSFCSLSSWRLALLVTSLELLQLDKYKNYDFGRCPRVYCSGQPCLPVGQSDIPRSSTVKIYCPRCEDLYYPRS

SEQ ID NO: 23 - protein, 59 aa, Glycine sp.
SGSDGDDTSWISWFCNLRGNEFFCEVDDDYIQDDFNLCGLSSQVPYYDYALDLILDVES

SEQ ID NO: 24 - protein, 98 aa, Glycine sp.
LIESAAEMLYGLIHARYILTSKGMAAMVFFSLLSCFLILYCGQNWHLMTLDYLQLHKYKNYNFGRCPRVFCSGQPCLPVGQSDVPRSSTVKIYCPRCE

SEQ ID NO: 25 - protein, 50 aa, Glycine sp.
WISWFCNLRGNEFFCEVDDDFVQDDFNLCGLSSQVPYYDYALDLILDVES

SEQ ID NO: 26 - protein, 194 aa, Glycine sp.
SGSDGDDTSWISWFCNLRGNEFFCEVDGDYIQDDFNLCGLSSQVPYYDYALDLILDVESSHGDMFTEEQNELIESAAEMLYGLIHTRYVLTSKGMAAMLDKYKNYDFGRCPRVYCSGQPCLPVGQSDIPRSSTVKIYCPRCEDLYYPRSKYQGNIDGAYFGTTFPHLFLMTYGQLKPQKPAQGYVPRVFGFKVH

SEQ ID NO: 27 - protein, 184 aa, Glycine sp.
ISWFCNLRGNEFFCEVDDEYIQDDFNLCGLSSQVPYYDYALDLILDVESSHGDMFTEEQNELVESAAEMLYGLIHVRYILTSKGMAAMLEKYKNYDFGRCPRVYCCGQPCLPVGQSDIPRSSTVKIYCPKCEDIYYPRSKYQGNIDGAYFGTTFPHLFLMTYGHLKPQKSTQNYVPRVFGFKLH

SEQ ID NO: 28 - protein, 57 aa, Glycine sp.
PLIAFGQFLNFRVYQLLGETGTYYGVRFGKTIPWVTEFPFGVIKDPQYVGSIMSILA

SEQ ID NO: 29 - protein, 39 aa, Glycine sp.
GLISVIVWVVAEIPQILTNYRTKSAEGLSVTFLITWIIG

SEQ ID NO: 30 - protein, 39 aa, Glycine sp.
GLINVIVWVVAEIPQIIPNYRTKSAEGLSVTFLVTWIIG

SEQ ID NO: 31 - protein, 57 aa, Glycine sp.
GFISLICWGVAEIPQIITNFRAKSSHGVSLAFLLTWVAGLVSLSTFLHFLIVLSKSY

SEQ ID NO: 32 - protein, 39 aa, Glycine sp.
GFISLVCWGVAEIPQIITNFRAKSSHGVSLAFLLTWVAG

SEQ ID NO: 33 - protein, 97 aa, Glycine sp.
GLTSLVFWGVAEIPQIITIFRTKKSHGVSLVFLLTWVAGDICNLTGCILEPATLPTQYYTALLYTITTIVLLLLIVYYDYISRWYKHRQKVNLVRDH

SEQ ID NO: 34 - protein, 59 aa, Glycine sp.
YEKHSTFGQWLGWLMAAIYISGRVPQIWLNIKRSSVEGLNPFMFVFALVANVTYVGSIL

SEQ ID NO: 35 - protein, 39 aa, Glycine sp.
LTSLVFWGVAEIPQIITIFRTKKSHGVSLVFLLTWVAGD

SEQ ID NO: 36 - protein, 29 aa, Glycine sp.
VPQIWLNIKRSSVEGLNPFMFVFALVANV

SEQ ID NO: 37 - protein, 39 aa, Glycine sp.
QFMVLQVKTTNPKKYCVRPNTGVVMPRSTCDVIGFFFSL

SEQ ID NO: 38 - protein, 31 aa, Glycine sp.
CLFFKVKTTNPKKYCVRPNTGIVTPRSTCDV

SEQ ID NO: 39 - protein, 47 aa, Glycine sp.
TCNFDLGVHFMVLQVKTTNPKKYCVRPNTGVVMPRSTCDVIGFFFSL

SEQ ID NO: 40 - protein, 122 aa, Glycine sp.
LHIEPAELRFVFELKKQSSCLVQLANNTDHFLAFKVKTTSPKKYCVRPNIGIIKPNDKCDFTVTMQAQRMAPPDMLCKDKFLIQSTVVPVGTTEDDITSDMFAKDSGKFIEEKKLRVVLISP

SEQ ID NO: 41 - DNA, 265 bp, Artificial
  Hairpin RNAi construct targetting Chlamydomonas sp. CDS1p Homologue
atgcgaccaaaacaacaagctcaggtcaccgccgcggaggacaccgcgtcggagccggagccggagcccaagcccgtggatcgaaagtcgaggctcaggtccttccgcgtacgcaccatctcctcggtcttgctcatcggcgggttcatcggcatcatttgggcgggtcatgtgccgcttatgttcttcatcctgctactccagttcctggtggcccgggagctcttccgcatcgcctacatagcggagaagtccaaacgcagcc

SEQ ID NO: 42 - DNA, 22 bp, Artificial
  LBa1 primer sequence
tggttcacgtagtgggccatcg

SEQ ID NO: 43 - DNA, 22 bp, Artificial
  LBb1 primer sequence
gcgtggaccgcttgctgcaact

SEQ ID NO: 44 - DNA, 1856 bp, Arabidopsis thaliana
ttgctctctcttccagagaaaggggaaaaaaaaaaaaaacagattcctgatattttctgcttcgcttcgctttgctttggcctttgggtttgctttgccttttatctccacaataataatattcgttaacctctcctcgttctcgtcgctgtttcttcccgatctttgctatagttctggattcaaaatctttcttccccaatagatgtttgtctcttaatttttgatttttctgcaaaatcaaatttttaagagctatggaggaagagaatgttactagttcaccttcaacaccggtacataggcttaggcatcgaagacgttctaatgaggttgttacggatggggataaagtgaatgcaagtcctttgttggttaatgatcggaataagtacaaatctttcatggtccggacctactctacgttatggatgattggtggttttgttttggttgtctacatgggtcatctttatatcacagctatggttgttgttattcaaatatttatggctaaagagttgtttaatctgctgaggaaagctcctgaggataaatgtctcccctatattaaacagctcaattggcacttctttttcactgccatgcttttcgtttatggacggatccttagtcaacggttagccaataccatgactgcagatcagttcttctatcggctagtcagcggcttaatcaaataccatatggcaatctgttacttgttgtatattataggtttcatgtggttcattctcacattaaagaagaagatgtacaagtaccagtttggccagtatgcttggacccacatgatcttgatagtcgtgtttactcaatcgtcgttcactgttgccaacatatttgaagggatcttctggtttcttctcccagcatctttaattataatcaacgacatcttcgcctacattttcggtttcttctttggaagaacgcctttaataaagctgtctccaaagaaaacatgggagggattcatcggagcctctgtgacaactatcatctctgcatttgttctggcaaatattttgggtcgtttcccttggttgacatgcccacgacaggatttgtccactggctggctacagtgtgacgctgatccattgttcaaacctgaaccttttgctttacctgcatggattccagaatggtttccttggaaggaaatgacgatcctccctgttcagtggcatgctttatgcctcggtttgttcgcttcaatcatagcaccttttggaggatttttcgctagcggtttcaaaagagcattcaaaattaaggactttggtgatagtattccaggacatggtggaatcacagatagaatggactgccagatggttatggcagtatttgcctacatatatcttcaatcatttattgtctcccaaagcgtttcggttgacaaaatcctagaccagatattgacgaaccttacctttgaggaacaacaagcgctcttcgtgaagttaggacaaatgctgaaggacaagctctcataggctccattgccaaaagtttcttttattcagattctgattctccaagaacaggttcctctggaatatccttttatctttgtccttactagagacaataaatttaaaattggattaagatttgttgtttcctgccattgtaaatcctctcgaaatttgtcgaaaaaaatagaagtttttttttttaagtcttctcattggatacaaaaggtttgaaggaagttgtatctactactatctacttatttagtagctttggtggtttttagctgtattttgtatattggttaaaatgaaacaacatttggtttgtttgtctcatgattcgatttgttc

SEQ ID NO: 45 - DNA, 2870 bp, Arabidopsis thaliana
tgttttaataaaagcatttattggtgagagagtttggagtacatgttattgatataagtacatgtcattgattgattcagatgagttttctacttatatttgcaaattgattctaaatttactcgactttttttttggcttatcaatgatttttagttgccagagtctttctatcttttttttgtattctgacgttagactaaggtgatgctagatgtactcaagttcaattttaaattttcacaggtagtcagtgtttttgtttcgaactcttccaacatgcagaaggaaattgctggtgatgctccatcagcaccaaccactcgtgttcggcatcggaagcgaaacagcgatgtatgtttgccattattcaatattttagctgtcaaattaaatcggaattcacattgtttttggcttagttaagttgcttttgttgtataaggatttatttggtccttttctacttatgtgatgtagaagctattctaagagtacataagctacttatttgcttcattttggttgtaaattcaatcaaatttctggctttttaggttggtgcaggggcaggcaaaccaaatggaaaccatttacttgtcaatgacagcaaaaaatacaaatcgtttctcattcgagcatactccactttctggatgataggtggttttgctcttatagtttacctgggtcatctctacattacagccatggtggttgttattcagatattcatggcacgagaactttttaatttgctgagaaaaactcatgaagataaacagctccctggttttagattactgaattggtaattatactgtctcacttctctcaacattctgagttattttttgttttgcttttatgtgaattatgctgaaatggtatcatattcttagtacttttctatttgttgaaacgattttgcatttgactactgattttgtttctgcaggcacttcttttttacagcaatgctctttgtatatggtcgaatacttagtcaacggctagtcaacactgtgactccagacaaagtcctatatcgactggtcacaagcctcatcaaataccacatggcaatctgttactccttgtatatttctggttagttgttatatgaccttttgggtttactgattactgtgaggagagcaatgatttttgttcactagttttacttaacataatctctcctttttttgctgtggaattgtaggctttgtttggtttattctcacgctgaaaaagaagatgtacaaatatcagtttagccaatatgcatggacacatatgatattaattgtggtgtttactcagtcctcattcactgtcgccaacatctttgaaggaatcttctggtagtcacctattccttttaacgcttcttacttgtttgctccgtgtttagttcttgtgttattttctcttgtatcttggttatgacctgactaaaagtctatttactatactggttgctggtgcaggtttcttcttcctgcatcacttatcgtcatcaatgacatatttgcatatatctgtggtttcttctttggaagaacaccgttgatcaagttatcaccaaagaaaacatgggagggtttcattggagcttctattaccacagtgatttctgcattcctggtaagcttgttttatgggctaataagtatatgttttttgacgagtagtaattgattaataatcttgcttgtttttgtagcttgcaaatataatgggtcgtttcctgtggctgacatgtcccagagaggtacttgaaagtttaagtcatgtcctgccttttttcatacaagaacctttacaattatttactaaccattatgttattggggttatgaatacaggatctatcgacaggctggctcctctgtgaccctggtccactgttcaagcaagagacacatgctttaccaggatggatttctgattgggtaaagtctagaattgaattgtacaatactaaacaattactcgagcaactttattgcttcttaacgtgaggcgtttactttacagctcccttggaaggaaatttcgattctaccagtccagtggcatgctctatgtcttggattgttcgcctctataatagctccttttggtggcttctttgccagtggtttcaaaagagcattcaaaatcaaggtgggtttcagatgatattcagtataaaaccacaatatctatgcaacattgattcactgttaaatgttctccaaccttgcaggactttggtgatagtattcccggacatggcggaattacagatagaatggattgtcaggtatataggcgcattgacttcaatacattcttattactttcttaaagactttcaaacatcactatggtttagaactgtattagatctgatatgatatgtcttttgggtttctagatggtgatggctgtttttgcctatatatatcatcaatcgtttgttgtacctgaagtcctctctgtggataagctcttagaccaggtaaaagtttactagtagaagaaccaacacaatgtacattaagtaagatctcttatttgacttgtagtgatttatactcattacagataatcacaagcctgacattggaagaacaacaagcgctactcgtgaagcttggccaaatgttgcaggaaaaggttattggatcttagatcatcacaacccgtcattgtataccatattactctgcttccgtgttacgtctgaaaattattcaaaagaaagagaaaagaaaaaaaaaagtagatttataagtttttttgtttttctcattgtggaattcctctgtatagtactggtacatgctttatgaaagaaaagaagctcactacaattctcttac

SEQ ID NO: 46 - DNA, 867 bp, Arabidopsis thaliana
atgattcgagatgatttgtcactatctcttggtatcatcagtgttatcagttggagtgttgccgagataccgcagattatgactaactacaatcaaaagtccattgaaggagtctctataaccttcttaacaacatggatgcttggtgatatctttaatgttgttggctgcttgatggaaccagctagtcttccagttcaattctacacggcagtgttgtatactctggctacacttgttctctatgttcaatctatatactacgggcatatttacccgcggttgatgaaaaaccgaaggaatcatcagatggttgatgtagaagagcctttgcttcgtgaagaagctaagcgtccttctaccaaatcgttgctatgtgtggtctcggtgttcttgtttcttggatcattcaacgtattgagtggttcaagaagtatggatttgagagggaaagatagagtgtttgtagttggaggagcaggagcaagaaagcttttggaggtctctagcggtaacttaggagaaaacaacaatattggaatgtggttgggttgggcaatggcggcgatttacatgggtggaaggcttcctcaaatatgtatgaatgtaaggagaggaaatgttgagggattgaatccattaatgttcttctttgcattcattggcaatgtgacttatgttgcaagcatactagtgaacagcgtcgaatggtcgaaaatcgaaccgaatctaccatggctagttgattcaggaggatgtgctgttcttgattttcttattctgcttcagtttttctactttcactgtcgcaaagtcgaggcagattccgtcaagaagaagcatgaaaccggtgaagaagctgtctaa

SEQ ID NO: 47 - DNA, 1951 bp, Arabidopsis thaliana
gaacaagattcgacaaaagctaaaaataaaataaaagaagaagcaaaaccctagagctcaaggtaatcgccgtcgatttctcaatctctcaccggcgattacatgccgatgaaagttgtacaatagctgctgtaagagagagatttatcaggtatttttagaatcatgtatagagacagaggaacggtgaattcgagacctgaagttgtggatcgtaaacggatcaacgacgctcttgaacgtccttctccttcaacctctcgtcaagttaacggcaaaggcaaaggaaccgttacggcggcgacgacgacggcgaacttgattgggaagcagcagtcgaataacatcaaccatagagattctcgttctgcttctctctcgaagaacaatacggtttctgatggttagtctctcttgctctattgaaatcgaatctacatttagaaacagatacttaaccctagagtttcctgatttgggaatcaagttttgatctttatgtttagtatcttatctccatgagctgaattaagcaaattcctttattagcctatggaatttttgatgtgaatagatgaatcagacacagatagtgaagaatcagatgtgagtggttctgatggagaggacacttcatggatatcttggttttgtaatctaagaggaaacgagttcttttgtgaagttgatgatgattacattcaagatgacttcaacttgtgtggactcagcagtctagttccctactacgagtacgctcttgatttgattttggatgttgaatcttctcaaggtaagttgttatccttttattagtttttgcggtttagctaggtacaggcacagcatctgcatttagggatagtagggaaaccgatatgggttcgcttttacgattgtgtctcttgaatgtttgatttggattggtcggtaacaggagagatgtttacagaggaacagaatgaattgatcgagtcagcagctgagatgttgtatggactgattcatgctcgttacatattgactagcaaaggattggcagcaatggtaaacaaaatccttaaacactcatttgcatgtctttcacattctataaaagattcacagattgattctgtttatttgcagttagacaaatacaaaaactatgactttggaagatgtccaagagtttattgctgtggccaaccttgtcttccggttggtcaatcagacttaccgcgatctagcactgtgaagatatattgcccgaaatgcgaagacatttattacccacgatcaaagtatcaaggcagtatccttttttctacagtttcacttctgctgatatagagttatataaatgcagttctgaaaaactttgatgtttgattcttattgcatgaagaaatgatttttcttttgagtttctagtgatctttggtgttacctttttttgcggttttccttaaaagaatgaacagacattgatggagcttactttgggacaacgtttccacatctgttcctgatgacttatgggcatctcaagccagcgaaggcaacacaaaactatgttcaaagagtgtttgggttcaaattacacaaaccgtgaaggaagtttgacgacaaaacccaaggatcaagatcatggcgttggaatggaactgagaaaagaagagacgtgtgtgaacagaaacggaaaataaaacgcactgtaatcacacgaatggtgaagagagcattcatgtctctatttgtcttcaggtacatacacattagaagaaagcaaaagcatggttagaaagaaaaaaaacacgaaaatcttttaatgagtttgttaaacttgaacatgtatttttctaccttctgtaaacttttttattttgtttcttttctcagtgtctgatttagtcatttatcatttaaaaaagtatccattaaaatttatcaaagatttcaatttgtgaag

SEQ ID NO: 48 - DNA, 3724 bp, Arabidopsis thaliana
acttggccttcactaagattagtttttgcacttctctctttctctcgatcgctgcttcttcgtcgctcaatctcaagctccgttccgttagttcttcgtcggagagacggtaaggtttccttccgatttcatcattctcgttgatcatcttcttctctggtttttgtttttgtttcgaaactaggttcctcttcctcgagtttcctgatttttgtctttagaatctgctttagaaatcgaaatcgctagggctcgggaagatctatgtgatctgagtttactattggctaccggatcccgaggagtcgctcctcgtaaacgtcgtcgttttatttcgcccttttgttttaatactatcaggcctgtggtggcgtaactagatcacactgggggactaggacgatttggttttccggctagtcgaaagcgcgttgcccaaaaaattgtgtcggtcggatcgctgagaaaaaacgagcgcattaaggtaaaatatagtcacgtttgagtcttcgggcgagttcgtatcgtcagatttgagaacgcgtcgtcgacgagttagtgcggtggtttgtttatggtgacggaagacgagaatccggactcgaaagagtgtaacagagaaacttttctgattgttgactgtttggttagatttcgtcatgttcccaatgggagagcgggtttatgattgtcccaaatagagtatagatctgagtttgtttttcgctctagtgtcgacagtttcatacttgaatggttctgggctgatctttctcttattatttttttgttgaaataggggtggagagaatcctgctgcaagtggtagctgtttgatacttgagtcattttgtcacagcaacatgaatatgccgctgttggatattcaaccacgaacgttgcagtttgcaggtaagatataaggggactgattcttatcaattgtaaaatgatcatttttaagttccagtgagtagacactgtggtcccgttaatagtttcttatttttttgtaaaatgtggatttcatagcgagggtgtgttattccttaagcaaagtggccaacatgttccagttatgaataccgagttttctgaattctcactatttcgttagaattctatcatagtggtgctattgtttgtgggtctattctcctgtaagatgaattgatgtgatgttaatgtggctgttgtttggaggaatcttctaactctttcctgctactggatttgtatgtgctgacattctgtgatgtggtttcttacctctcatatcgattctgaggatctataataaaatatggaagttgtaaaaggaatatctttgccttggcattgttatccttgtaatactaactggtttcatcttttgtagttgacctgaagaagcaaacttcctgcgtggtacagctaaccaatactacccatcattatgttgcatttaaggtctaaagaagattcttctcaaactttttctctttctttctggcttcaggatttcgaatatcttataaaatttctgagaaaaagtattctttctgtatacaaacctaaactagatgtggtttttgacaggttaaaaccacatcaccaaagaagtactgtgttcgtccaaatgttggtgttgttgcacctaaatcaacctgtgaattcactggtatgcctactttttatattcttcccagcaatgcttgaatttcttgcaccgacgttcttaggccgatgtgataaactgttgcatgatgtctacatggggcgggaaatgaactggaatatatgtatagatctgtcctttgtcccttggacttaaacagcgtttggacattgttttaagatcctaggcttttagagtttagtgtagttgttcaagagagaaggtatctgctccggatggaattattaaagttcaagttattactcattacgtggaacctgcatttttgggtttgtttgagcattcaaaattttaaactggttgatgatgaatgggttcttgaaattcaaattcagtcaatgctttgggttattgagttgtatatgttatttatgcagtcattatgcaagcttttaaagaaccacctccagatatggtttgcaaagacaaattcttaatccagagtactgctgtatctgcggaaacaactgatgaagacatcacagctagcatggtaagcattccaacacttgttgaccttctttatcattaatttcagttatttggtctgtcaagtatatatgtacactcatttattcttggcagttttcgaaagccgaaggcaaacacatagaggagaacaaactcagggtcacgctcgtaccgccctctgactcacctgaactatctccaatcaatacaccgaagcagggagcagtatttgaagattccatcctcaaggatcgtttatatagccagtcagagaccctagctccgccacaatatgtaagaagagtatttaccccttttcgcaacaacttttatgatttgttagtaagatgaagagtaatttgccaagcagtgagtgtagttatgttccagcaagttcattttagcttagcttataatcacagttattgggtgcaatagggattttcttgtagttatttgtttattctcaaaatttatttttctaggagggtgagattgtaaaagagcctaggatggttggacatgatgagttaaaggcagccgacaatgcaaaggagttaaagacaccaaaaatggccacagtggattttgtggaagatcgatatactgctaatgacttgaaggcaactaaagatagctacgattcatcgaggatggcaaaagaaactggattcgatccaattaggtctcataaggatgcggatgatggaagggcgattaaggcaacaactaatttggacgctcccatgaagaaggccatggatctacctagggatcaagggtttactaatggaatagctgttgacagtgaaccaaagatatctaaggagagagatgttgtccagctgcagaaaacagatggccagaatgtcagaggattggatgaacttaagttggttaaagacattgaggagatgaagctgaaagtggatgctcttgaatccaagttaaaacaggtctgcttatcaaattttaaaatccataacttccgtgctatatgtaactgtgctaaagagtttaaatctggtgcaggctgattcaaccatttcgaagctaatggaggagcgatctataagctcccaacatagacaaagcttgcaacatgagctggtgagaatgctagtgagaaaacatagctgtttctttctcctacaatcaaaacagtttcataataacgaagcttgtattttgatgatgatgcaggcggaactgagaacgaagaagatagtgaaagaagtgcacaatgggttccctctgctgtatgtatgcgttgtggcattcattgcctatgttatcgggcactttctgcgcacttgactgctcgctcggtgtctatatcatatcaagtgtgcagtaagggatatatagacaaagaaggtaaaaggattagatttcgtctgtttgggtttgctttgtgggagaggatgaatgatttctccgacacatttaagatggaatatgatagactgtatagcattggtgttagttaacacaaggacacaaggacatatggtatgatgatatgctttgtttctctgcttctcttactaatttgaagctgttggattgatttgt

SEQ ID NO: 49 - protein, 421 aa, Arabidopsis thaliana
MEEENVTSSPSTPVHRLRHRRRSNEVVTDGDKVNASPLLVNDRNKYKSFMVRTYSTLWMIGGFVLVVYMGHLYITAMVVVIQIFMAKELFNLLRKAPEDKCLPYIKQLNWHFFFTAMLFVYGRILSQRLANTMTADQFFYRLVSGLIKYHMAICYLLYIIGFMWFILTLKKKMYKYQFGQYAWTHMILIVVFTQSSFTVANIFEGIFWFLLPASLIIINDIFAYIFGFFFGRTPLIKLSPKKTWEGFIGASVTTIISAFVLANILGRFPWLTCPRQDLSTGWLQCDADPLFKPEPFALPAWIPEWFPWKEMTILPVQWHALCLGLFASIIAPFGGFFASGFKRAFKIKDFGDSIPGHGGITDRMDCQMVMAVFAYIYLQSFIVSQSVSVDKILDQILTNLTFEEQQALFVKLGQMLKDKLS

SEQ ID NO: 50 - DNA, 4024 bp, Brassica rapa
atggaagaagagagtaacgttaccagcagctccccttcaacaccagtacaaaggcttaggcatcgaaaacgctcctccactgaggttcctttcttttcctttttttcagttataactaactgttgcatatgctctttgaatctgttatgactcggttgcattagttagttggacatctgttatatatgctgcggtgtttagcttagtcacatttactaaggtctctccccacgagtccgttactattttatatatcttgtgttcttgtaaccttctgaaaagttgcttccaataataaagatctctcttttacttgtgaacgtagccaatttgatgaaatctctgtgttttttatttacactcatggtaaattagctcatcaatgcaaaaaaaaaaaattaaaagtaagtttgatgagtttttttttgtcttggtttgtggtatttggttaggttcttgatggggacaaagtgaatgcaagccctctgcttgttaatgatcgaaacaagtacaaatctttcatgatccggacttactctacactatggatgattgcgggtttcgtcatggttgtctatatgggacatctttatatcactgccatggtgcttgttatccagatcttcatggccaaagagctctttaatctgctgaggaaagcacctgaggataaatgtctccctgggatcaaacagctgaactggtacagttattagactattggctcagaagatgtataatgttgttcttaacccgtcttgctttgtttgttttcctgcaggcacttctttttcactgccatgcttttcgtgtatggcaggatccttagtcaacggttagctaatactgtgactgctgaccagttcctctacaggcttgtcaccggcttaatcaaataccatatggctatctgttacttcttgtatattataggttactctcttgctccatcattttgtttctttttggtttttaattatctgcgaaaatgggtcttaatcccttgcttatggagtctctgctttaatgtataattataatgaattgcaggtttcatgtggttcattctaacattgaagaagaagatgtacaagtaccagtttggccaatatgcatggacccacatgattttgatagtcgtctttactcagtcctccttcactgtcgccaacatattcgaaggaatcttctggtacgtttttaacaatttcagcatataataatcttgaccaccttgcttcctacactttcacattttggccccaatctgatttacttttcttctttgttaaaattgtaattgtcttcttgattgatcagtttctacttttgaattacaggtttcttcttccagcgtctttaattataatcaacgacatctttgcttacattttcggtttcttcttcggaagaacgcccttgataaagctatctccaaagaaaacatgggagggcttcatcggagcctctgtcacaaccatcatctctgcatttgttgtgagtattggttacctctggccctattcatttcattgtatcttcatcaaacctctgcctcaatatgttctgttcgtgtaatgtttcagctggcaaatgttttgggtcgtttcccatggttgacatgccctcgtcaggtgagtctttaagaaacgaaattgacacttatcttaaacctgatcgttgagggagcttatagtagatctgtgaatcattctgcaggatttgtccaccggctggctacaatgtgatgctgatccattgttcaaacccgaaccttttactctacctgcgtggattccaggatgggtaagctttagtataatgttgcattgcttgcaagaaactcacccatgttcaagaagacatattataactgttatcagtggttcagactttctggaaactttctgaactttttactacacttttgcagtttccttggaaggaaatggaggtccttcctgttcaatggcatgctttatgcctcggtttgttcgcatcaatcatagcacctttcggagggtttttcgctagcgggtttaaaagagcattcaaaatcaaggtttgtattcaacaaccagccaaatttgagctaagcttcgcttatgtgttaagatacacatctaacttctaacaaaacctttgattcataaaaaatgtaatcgcaggactttggtgatagtattccaggacatggtggaatcactgatagaatggactgccaggtaactctagtgaaactagtatgagaaattgcttgagagagctaatttaatcttttatcacaaatttaaagtatttatgaaatagttatttttaaagagtaaatttgaaatgtggtatcaaacttgtggtaaaattgaaaaaatttgaaggctatgggttcatttgaaaatgctaaaatgtgaaaaaatgcaaaaccaaacagggcatacatactatttacttgatttcattttttttacccccatctttgagcatccatattcagactttatctttttttaatgtcacagatggtaatggcagtatttgcttacatatatctccagtcatttatcgtctcccaaagcgtttcggttgacaaaatactggaccaggtagtttaacttgtctatttacatttggacttaattacgttttgcaacgaatcaattcaatcatgtgatgtcgtttcattgtagatattgacgaaccttagcttcgaggaacaacaagctctcttctctagattagggcaaatgatcggtaactcatagtgtccactgcccaaaacttctcttcagattctccaagaactctggtttatccttttatctgtatcagttctagggacaataaatttgaaattggattaagatttgttttctggcattgtaaatcctcttgaaggttgtccaaagtagaagttttttctttaaagtcttgtcatttgatacaaataggtttgaagttgtatcttactatctacgtatgcagtagcatcggtgcttcatttgtattttgtatatggttaaagtttaaacaaactatttttttttctgtggttccacgactcgatttgttaggaaagttggtgcatataaggtgatgattgttttcttactttttaaattgtagtttttgatttttcagtaaattttagtttttagaaaacgttgttgttagttttttttttttttttagtttttagtctttacttataaaattaataaaataaaaatattattaaagtaaaatgtttattctgaaaaaaatgttatcaaaaactgtaaatacgaaataaaattactgtaaactctatacatatgcagaaatttgatctcaaatcaaaattaacatctttgaaatttgatagcttaataaaactatgtagagccatcgttgcaataactcttatctgaatatgtatgttgtatctttgattctctcttagaatctgtcattttctatcccacatactaaaagtctgttcaataacatatcgtaatgaaaaatctcgtttgttaaatattttttataactaccaggtggtttaggaaaaacgttcgcagatgtgatcatgaatcgaaccagatcgaaacattcagctgtcgacggacaggatgatatctttctctatatatggagtaagaaatctaagtcgacgaggataactagaataccatatataatactttttgaaatgtgcagttaaaaattgatgaggtctcttcattgttagtgctagaacttttgcctcataagatgatctcggaataactatatgcatatgtaaacaatatgtatagctaaatattcatactcaagtcggttttattccatatttgttgaatcttttcttacaataatgacatgaacatgtgttctatcgattgcactgataaactctttaaagtaaggttagtaatgtttgtcaaaaagaaaaaaagtaaggctagtaatgtttttgtattgtaatcttggatgagctaccgttcctgtcgactttaaagaagcgagagaaattgttgaaactctatga

SEQ ID NO: 51 - DNA, 547 bp, Brassica rapa
atgtttacgggaggagggacggttggaccgaaagccgaggtggtggaccgggaagggattagcgaagcttatgatgttaatcggtccgagggcaaagaaagagccggagctaactctgtgttgatggggacgcagatgcatgattctcgttctgctactctctcccggactgacattttccaaggttagattcgattatcgctattgtagttgcagattgtttgattcgcttcgaattggtatttagggctttgatcaatggttatagaacccataactcatatttggaattatgctgtgtaaacaaacacataaaagctgtttctctgtgtcctttagctgatgatatcgagaaaatcgatcttatttgcttaaaatcgatttataatgatgaactaatgtttttgtgggcatttcttagctgtagatacctcagaaggatctgaggtcagtggttcggacgaagaagacttggcatggactacctggttctgtaaactacctgggaacgaattcttgtgtgaagtcgatgattgtttcatcttag

SEQ ID NO: 52 - DNA, 2303 bp, Brassica rapa
atggtgtatgcacgttactgcttgaaggagaagaagacatgtgtgagatgggtggagagatacttcgatgactgtctctgtaacctgaacgacgaagtctcgttcgcacttggaatcgttagccttatctgctggggcgtggcagagattcctcagatcattaccaacttccgtacaaagtccagtcatggtgtctctctatctttcctcctcgcttgggtcgctgggtccgtacactccatcatcttcagttcttattaattttatatttattataaacgtaatgaactctagatctctctaactttgttatttttacgactttttatcaatcggtgcagtgatatcttcaatctcatcgggtgtcttcttgaaccagctaccgtaagttgcacatattaagaccactatagttttgtttaaatatcgattgttgttttctcactttgtcttttcctttttttattattgtagttgccgactcagttctacacagccttggtaaaaccgctgtctactcgccgcataaacatctgtcgccataaaagcttacgtgtcattttcattaaattgtatatgtttaattttgttcttgcttaacttatttataattttgtaatttttttcaactggttctctagctttacacagtgagcaccgtggtattggtgatccagacaatttactacgactatatctacagactttgcacacatggacgcaccaagatctgtcaaaaggtactaattgtattaattttataaattataaaccaactacactattagttatattgtgtaatactctgatgcgggtgtcaaactatgttaaggacgaagaagacgaagagaagaaacccttaaacccaccgaagacgatgggatctgccatttccatcccaggggaatcttacaaagcttctcctcggaaagagttctattacacgtacgtttcattatttaattcacttttaacttgattatgacttgagttaaaggtagtgacaaaagcaaatcatttactatgaactaatgcattattttactcctacatatatgcatagatgttagtatcatattgtaataggatgtgacactttaccttggtactggtttttccttttcaggtcagcgagatcattggccggcagcggaacaccgcctttaagaacatcatattttcgggtggctaagagtggcccttcggctatgacaatagatagtggttcttcctcggacgaagatgagacaatgtcaacacttcctgttgtgacggctaagaccattacccaaccaaggccaatccctagacaggtttttgattattttttttctcattggaaactaaaaaccattttatgcaaaaaggaacaacgattgacacgttttgtacttcttcctctcaggctggttttggaacgttcttagccgcatccgttagccttccattgcaggccaagagcctagcagaaaagtatgcacatgcttcaagcagacggcttctgaatgtgtgtaactgcttatataatatttcgctacttcttattgtataaatggaattatttatttgctcaaaaaaaaaaatggaattatttcaaaacattatttgttatctcgttataggaaagaatagttgagcatagcgcattgggacaatggttgggatggctaatggcagccatttacatgggcggccgcatccctcaaatctggctcaacgtaagctactattttacaagtcttattgattcttaattttcaatttctctttgcttattgtttatgatagatactaatcttatcttggttcggccaataattttataaaactagatcaaacgaggaagcgtcgaggtaaatatctaaaccagacaaatcaaaaagcattgcaataatatatgatctaattaataatatgttttgatttttggcagggtttgaatccacttatgtttatcttcgcgcttgtagccaatgcaacgtacgttggtagtattcttgtcagaacaattgaatgggatagtatcaaagcaaatctcccttggttgcttgatgctattgtctgcgtcctactcgatctatttgtatcctttaaaaccatttacacagcgccaattaaattttaaaatgacatcccattcccactaagttttattttgctaccttaattcaagacaaataaaacagatcatattgcagtatatctactacaagtattgcaggacgaatagctcaaaaggcgaagaagaagcagaacacggctacggagactatgtggaagcaagcaaaacttttgtttcgtga

SEQ ID NO: 53 - DNA, 1257 bp, Brassica rapa
atgacgacgggagatctggtgaatatccatcctacagagcttaagttcccttgtaagttttttatttttatttttaataatcgaatcaaacaaatgtttcgacgatgattggaaaaattcgaattttgattggattggtccctgttgttgaaattgaaattgacatttgaaagttgagttgaagaagcaaagctcgtgctcgttgcaaattagcaacaagactagtactcaagttgtcgcatttaaggttaagacaacgaatcctcgcaaatactgtgtccgaccaaacaccggtgttgtcttgcccggtgattcctgcaatgttacaggtgatcaattgaattgtgtttttctttttcttttttaaaaaaatatctattatactgatcaattgaactatgtgtgtttgttccttactttagtgacgatgcaagcccagaaagaggcacctctcgatatgcaatgcaaagacaagttccttgttcagtctgttatcgtctctgacgctactacttccaaagatgtcctcgctgaaatggttttgttttgcttctcttttcatgtttaccatttccactctcctctctttagttactcttcattacattggttgttgcagttcaacaaggagcctggtagagtgattgaggatttcaaattgagggttgtttacatccctgctaatcccccttcacctgtccctgaaggctctgaagaaggcaactctccaaggaccgactttcctgcctctcaatttgatgaccacgtgagctactacgagcttgttggagagagagagagagagattttagtgagtgaagctaatatgcatcctctcttttcttgtttcaggtgtccagaacgctagaagaaacaagtgagaaatcctccgaggtacaataatgtcacacaagtcttttcttctaatctagactacacaatgcttctatttcattatcatatcattgtgtttttctcaggcgtggtccatgattcacaaattgacggaggagaaggcttgtgctgtccagcaaagtcagaagctccgacaggaactggtaagtgatttacttgtccttttgttttataaagaaagtaacacatatatttatatatatatatggctgttgtattcaggagatgctgaggagagaatcaagcaataagcagtctggtggtggtcattccttggtgttgatgctgttggttggtttgctaggctgtgtgattggatacatcttgaacgtccggacataa

SEQ ID NO: 54 - DNA, 3535 bp, Arabidopsis thaliana
tgctctctcttccagagaaaggggaaaaaaaaaaaaaacagattcctgatattttctgcttcgcttcgctttgctttggcctttgggtttgctttgccttttatctccacaataataatattcgttaacctctcctcgttctcgtcgctgtttcttcccgatctttgctatagttctggattcaaaatctttcttccccaataggtatacgcgaaaaaaaaaatccaacattttcttcttacttaaatcctcaaattttgtcacttgttcatgcttttaagtaatcaatttcttaatttcagtgttaagtttgtcacgaatcttatttcttttctgttgggttaatgaatagtcgattaaaaatcccagctttttcaatctgttattgctaagtgattaggaatctcagatgttgacgttcactgatagggttttagtcaaaattcctaatttcttcaagctttatagtaagaatcttaagttgtttgttttgggtacagatgtttgtctcttaatttttgatttttctgcaaaatcaaatttttaagagctatggaggaagagaatgttactagttcaccttcaacaccggtacataggcttaggcatcgaagacgttctaatgaggtttggttcgttatcttctgtagcttcattgaagaaaatttgaagtgaggtttacttagtgttgcttgtttgtgttaggttgttacggatggggataaagtgaatgcaagtcctttgttggttaatgatcggaataagtacaaatctttcatggtccggacctactctacgttatggatgattggtggttttgttttggttgtctacatgggtcatctttatatcacagctatggttgttgttattcaaatatttatggctaaagagttgtttaatctgctgaggaaagctcctgaggataaatgtctcccctatattaaacagctcaattggtaaagtcacaagctttttatcttctagttttaaaaaggctattggctcaggtgatgtaagatgttgttaagtcgtcttgtttgtttggtttcctctgcaggcacttctttttcactgccatgcttttcgtttatggacggatccttagtcaacggttagccaataccatgactgcagatcagttcttctatcggctagtcagcggcttaatcaaataccatatggcaatctgttacttgttgtatattataggtcagtcttgcttcaccgttctgtcctctttgtggaatttgattttctagcatcggtgaaaataggtcttgacgcttggcttagttgattcaccttacgtttaatgcttatggaatatgcctcgatgtaacaaatttcaggtttcatgtggttcattctcacattaaagaagaagatgtacaagtaccagtttggccagtatgcttggacccacatgatcttgatagtcgtgtttactcaatcgtcgttcactgttgccaacatatttgaagggatcttctggtacacatttaataatgtcaccatatgaagtttatgctttttcgactattcgtatattaagtttgctaaggaatcatctttttctatttgtcaccacattgaatcagactgttcttaacaaacatagcttcttacacttgcatctattttacttttttcttcgctgtaatgtaaattgtctacttctgatggcttccaatgcaggtttcttctcccagcatctttaattataatcaacgacatcttcgcctacattttcggtttcttctttggaagaacgcctttaataaagctgtctccaaagaaaacatgggagggattcatcggagcctctgtgacaactatcatctctgcatttgttgtgagtatatgttacttttgccaccacatggcacatcaaaatgcttacatcatttcatcatctttttcttcaccatgcttctgaaatgttttgtatgcgtaatgtttcagctggcaaatattttgggtcgtttcccttggttgacatgcccacgacaggttagtcattgagcacaaagaataacactaatcttgcagacgatcttttagggagtttatagtaaatctttgaatcatgatgcaggatttgtccactggctggctacagtgtgacgctgatccattgttcaaacctgaaccttttgctttacctgcatggattccagaatgggtaagctttggtatcatgttgcatcgtgtagaagaaaattatctatgttgtatattttggcgtttgtacttggtatatttctagtcatgaataagtgaatgtaggcgaactccctcagagtataacttatttcatagtgtattcttagataggttcagatcctggcctttttattagtttctgaaataggtttggattctagccattttatggaggttactcagttagctcttgttagctaacagatagtagtgttagactgtgcggttagcctttctgtttgaataatattgcatgaggtttctcaactttctagttcatttgtgcagtttccttggaaggaaatgacgatcctccctgttcagtggcatgctttatgcctcggtttgttcgcttcaatcatagcaccttttggaggatttttcgctagcggtttcaaaagagcattcaaaattaaggtcagtaatcaatacctccaaatatgagccaagcttcactttccatacgcatctaacctctaacaaaaccttgcttgacaaaaaccgtaatcgcaggactttggtgatagtattccaggacatggtggaatcacagatagaatggactgccaggtaactctaatgaaactagtatctcttcgtaagattatgattaggtctttagagttttcattcacaagattcgcattgcatattcttcttctctaactctcatctttaagcatatacataaaccagacctaatgttatctattgttacagatggttatggcagtatttgcctacatatatcttcaatcatttattgtctcccaaagcgtttcggttgacaaaatcctagaccaggtaaatgaacttctcgactttgtatcaaaactgaaaaatattcacttttgcactgaatcattgaatcaaacatgtgatggtttcattgtagatattgacgaaccttacctttgaggaacaacaagcgctcttcgtgaagttaggacaaatgctgaaggacaagctctcataggctccattgccaaaagtttcttttattcagattctgattctccaagaacaggttcctctggaatatccttttatctttgtccttactagagacaataaatttaaaattggattaagatttgttgtttcctgccattgtaaatcctctcgaaatttgtcgaaaaaaatagaagtttttttttttaagtcttctcattggatacaaaaggtttgaaggaagttgtatctactactatctacttatttagtagctttggtggtttttagctgtattttgtatattggttaaaatgaaacaacatttggtttgtttgtctcatgattcgatttgttc